(19) United States
(12) Patent Application Publication
(10) Pub. No.: US 2016/0215117 A1
(43) Pub. Date: Jul. 28, 2016

(54) PEROXIDE CROSS-LINKING OF POLYMERIC MATERIALS IN THE PRESENCE OF ANTIOXIDANTS

(71) Applicant: MITRAL TECH LTD., Or Yehuda, IL (US)

(72) Inventors: Orhun K. Muratoglu, Cambridge, MA (US); Ebru Oral, Newton, MA (US)

(21) Appl. No.: 14/389,852

(22) PCT Filed: Apr. 2, 2013
(86) PCT No.: PCT/US2013/034887

§ 371 Date: Oct. 1, 2014

Related U.S. Application Data
(60) Provisional application No. 61/620,202, filed on Apr. 4, 2012.
(60) Provisional application No. 61/756,595, filed on Jan. 25, 2013.
(60) Provisional application No. 61/794,284, filed on Mar. 15, 2013.

(51) Int. Cl. C08K 5/14; C08K 5/1545; C08K 5/134; B65B 55/16; A61L 31/04; A61L 31/14; A61L 2/08; B65B 55/18; C08K 5/159; C08J 3/24

(52) U.S. Cl. CPC C08K 5/14; C08K 5/159; C08K 5/1545; C08K 5/1345; C08J 3/247; A61L 31/048; A61L 31/143; A61L 2/081; A61L 2/087; B65B 55/18; B65B 55/16; C08J 2323/06

(57) ABSTRACT

Methods of chemically cross-linking antioxidant-stabilized polymeric material are provided. In one example embodiment, peroxide cross-linking can be used to improve wear resistance and the addition of antioxidant can be used to improve oxidation resistance of ultra-high molecular weight polyethylene. A balance between the amounts of peroxide(s) and antioxidant(s) in the polymeric material can ensure that enough cross-linking is achieved for wear reduction and that enough antioxidant is incorporated for improved long-term oxidative stability. In one example embodiment, peroxide(s) can be diffused into a consolidated polymeric material for cross-linking. In another embodiment, polymeric material is consolidated with a vinyl silane, an antioxidant, and a free radical initiator, and the consolidated polymeric material is contacted with water thereby forming an oxidation resistant, cross-linked polymeric material. Such materials can be used in orthopedic applications such as bearing surfaces in total joint implants, including total hips, total knees, total shoulders, and other total joints.

### CROSS-REFERENCES TO RELATED APPLICATIONS

This application claims priority from U.S. Provisional Patent Application No. 61/620,202, filed Apr. 4, 2012, and U.S. Provisional Patent Application No. 61/756,595, filed Jan. 25, 2013, and U.S. Provisional Patent Application No. 61/794,284, filed Mar. 15, 2013.

### STATEMENT REGARDING FEDERALLY SPONSORED RESEARCH

Not Applicable.

### BACKGROUND OF THE INVENTION

1. Field of the Invention

The present invention relates to methods of making oxidation resistant, wear resistant polymeric materials that contain antioxidant(s) and cross-linking agents. The invention also relates to novel methods of cross-linking the polymeric material by blending crosslinking agent(s) into polymeric material and diffusing crosslinking agent(s) into consolidated polymeric material. Methods of preparing polymeric materials with spatial control of cross-linking agent are also provided.

2. Description of the Related Art

Polymeric material, such as ultra-high molecular weight polyethylene (UHMWPE), is used in load bearing applications. In humans, it can be used in total joint prostheses. Wear of the polyethylene components over years is known to compromise the longevity and performance of total joints in the long-term. Radiation cross-linking has been shown to reduce the wear rate of polyethylene and thus extend the longevity of total joint reconstructions. Alternatively, organic peroxides have been used in polyethylenes for cross-linking. However, cross-linking is a result of the reactions of free radicals induced in the material, which can also result in oxidation, in the short term and through cyclic reactions, in the long term. Thus, the invention provides methods of containing antioxidants in cross-linked polymeric materials.

In load bearing applications where polymeric material is used, periprosthetic bone loss can occur because of wear particles released from the surface. Thus, cross-linking is used to decrease wear rate. A wear reduction of 90% has been shown with highly cross-linked (virgin, no additive) UHMWPE compared to historically used conventional, gamma sterilized UHMWPE when the radiation dose was increased to 100 kGy. Thus, high cross-linking levels are beneficial to reduce wear rates and periprosthetic osteolysis. The invention discloses methods of achieving high cross-link density and low wear rates by using cross-linking agents in the presence of antioxidants/free radical scavengers.

Organic peroxide cross-linking of polymeric materials with substantial oxidation resistance has not been achieved before. However, this invention discloses methods of introducing sufficient amounts of antioxidant(s) into polymeric material that also contains cross-linking agents. Subsequent to the decomposition of the cross-linking agent, which causes the cross-linking of the polymeric material and also deactivation of some of the antioxidant molecules, the methods described herein provide sufficient antioxidant activity remaining in the polymeric material to ensure long-term oxidative stability.

Organic peroxide cross-linking has not been achieved below the melting point of the polymer because methods such as extrusion and compression molding were used to decompose the peroxides during consolidation. However, this invention discloses methods of introducing cross-linking agents into polymeric material and decomposition after the consolidation of the polymeric material and optionally below the melting point of the polymeric material.

It is advantageous to have wear and oxidation resistant materials, for example, Ultrahigh Molecular Weight Polyethylene (UHMWPE), for total joint implants. Wear resistance can be improved by radiation and/or by using a chemical cross-linking agent. Antioxidants such as vitamin E have successfully been used in increasing the oxidative stability of polymeric materials.

Various methods of making cross-linked polymeric materials are known in the field. For example, U.S. Patent Application Publication No. 2008/0215142 A1 to Muratoglu et al. describes methods of incorporating antioxidants in radiation cross-linked UHMWPE. U.S. Pat. No. 6,494,917 to McKellop et al. describes the use of blended peroxides to cross-link UHMWPE.

Therefore, there is a need for improved methods of making cross-linked UHMWPE in the presence of antioxidants and for cross-linking agents for medical implants.

### SUMMARY OF THE INVENTION

Described herein are methods and approaches not found in the field for making cross-linked, wear and oxidation resistant polymers, and materials used therein.

Aspects of the invention include methods of chemically cross-linking antioxidant-stabilized polymeric material; in some embodiments the cross-linking is limited to the surface. It also provides methods to obtain a wear-resistant polymeric material to be used as a medical implant preform or medical implant using these methods. Peroxide cross-linking of polymeric materials can be used to improve wear resistance and the addition of antioxidant can be used to improve oxidation resistance; such materials can be used in orthopedic applications such as bearing surfaces in total or partial joint implants, including total hips, total knees, total shoulders, and other total or partial joint replacements. While radiation cross-linking of polymeric materials can also be used for the same purpose along with antioxidant stabilization, peroxide cross-linking and antioxidant stabilization offers a more affordable fabrication. One challenge with peroxide cross-linking of polymeric materials is the ensuing loss of thermal oxidative stability. We discovered how antioxidants can be used to prevent this loss of stability in peroxide cross-linked polymeric materials. Another challenge is that, just like it is with radiation cross-linking in the presence of antioxidants, in the presence of antioxidants the efficiency of peroxide cross-linking is reduced. Therefore, a delicate balance between the amounts of peroxide(s) and antioxidant(s) present in the polymeric material needs to be achieved to ensure that enough cross-linking is achieved for wear reduction and that enough antioxidant is incorporated for improved long-term oxidative stability. We discovered that peroxide(s) can be diffused in to a consolidated polymeric material for cross-linking. We also discovered that very high concentrations of antioxidant added to polymeric material along with an optimized amount of cross-linking agent can be used to achieve sufficient cross-linking and oxidative stability.

In one aspect, the invention provides a method of making an oxidation resistant, cross-linked polymeric material. The method includes the steps of: (a) blending a polymeric material with an antioxidant and a cross-linking agent; and (b) consolidating the polymeric material thereby forming a consolidated, antioxidant and cross-linking agent-blended polymeric material.

In another aspect, the invention provides a method of making an oxidation resistant, cross-linked polymeric material implant. The method includes the steps of: (a) blending a polymeric material with an antioxidant and a cross-linking agent; and (b) consolidating the polymeric material thereby forming a consolidated, antioxidant and cross-linking agent-blended polymeric material implant.

In another aspect, the invention provides a method of making an oxidation resistant, cross-linked polymeric material. The method includes the steps of: (a) blending a polymeric material with an antioxidant and a peroxide; and (b) consolidating the polymeric material thereby forming a consolidated, antioxidant and peroxide-blended polymeric material.

In another aspect, the invention provides a method of making an oxidation resistant, cross-linked polymeric material implant. The method includes the steps of: (a) blending a polymeric material with an antioxidant and a peroxide; and (b) consolidating the polymeric material thereby forming a consolidated, antioxidant and peroxide-blended polymeric material implant.

In the method, the antioxidant and peroxide-blended polymeric material can be further heated. The consolidation step (b) can comprise compression molding or direct compression molding the polymeric material. The heating can be done to a temperature T at about or above (i) a temperature T₁ at which one-half of a quantity of the peroxide decomposes in one hour, or (ii) a temperature T₁₀ at which one-half of a quantity of the peroxide decomposes in ten hours. The consolidating and the heating can be done concurrently.

The method can further include the step of machining the oxidation resistant, cross-linked polymeric material into a medical implant. The method can further include the step of packaging and sterilizing the medical implant. The sterilizing can be done by gas sterilization or ionizing irradiation. The sterilizing can be done by ionizing irradiation in inert gas. The method can further include the step of extraction of the oxidation resistant, cross-linked polymeric material. The extraction can be performed by contacting the oxidation resistant, cross-linked polymeric material with a gas, liquid, supercritical fluid, a solid, a solution, an emulsion, or mixtures thereof. The oxidation resistant, cross-linked polymeric material can be heated during extraction. The oxidation resistant, cross-linked polymeric material can be heated in a vacuum.

The method can further include the step of consolidating a second polymeric material including a second antioxidant as a second layer with a first layer of the polymeric material thereby forming the consolidated, antioxidant and cross-linking agent-blended polymeric material. The method can further include the step of consolidating a second polymeric material including a second antioxidant as a second layer with a first layer of the polymeric material thereby forming the consolidated, antioxidant and cross-linking agent-blended polymeric material implant.

In the method, the polymeric material can be selected from ultrahigh molecular weight polyethylenes, high density polyethylene, low density polyethylene, linear low density polyethylene, and mixtures and blends thereof. The polymeric material can be blended with multiple antioxidants and/or multiple cross-linking agents.

In the method, the peroxide can be selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

The method can further include the step of blending the polymeric material with the antioxidant such that the antioxidant is present in the polymeric material at a concentration of from 0.001 to 50 wt % by weight of the polymeric material, or at a concentration of from 0.1 to 2 wt % by weight of the polymeric material, or at a concentration of from 0.5 to 1 wt % by weight of the polymeric material. The method can include the step of blending the polymeric material with the antioxidant such that the antioxidant is present in the polymeric material at a concentration of from 0.6 to 1 wt % by weight of the polymeric material.

The method can include the step of blending the polymeric material with the cross-linking agent such that the cross-linking agent is present in the polymeric material at a concentration of from 0.01 to 50 wt % by weight of the polymeric material. The method can include the step of blending the polymeric material with the peroxide such that the peroxide is present in the polymeric material at a concentration of from 0.01 to 50 wt % by weight of the polymeric material. The method can include the step of blending the polymeric material with the cross-linking agent such that the cross-linking agent is present in the polymeric material at a concentration of from 0.5 to 5 wt % by weight of the polymeric material. The method can include the step of blending the polymeric material with the peroxide such that the peroxide is present in the polymeric material at a concentration of from 0.5 to 5 wt % by weight of the polymeric material. The method can include the step of blending the polymeric material with the peroxide such that the peroxide is present in the polymeric material at a concentration of from 0.5 to 2 wt % by weight of the polymeric material.

The method can further include the step of compression molding or direct compression molding the polymeric material to a second surface, thereby making an interlocked hybrid material. The second surface can be porous. The second surface can be a porous metal. The method can further include the step of machining the polymeric material before or after heating.

In another aspect, the invention provides a method of making an oxidation resistant, cross-linked polymeric material. The method includes the steps of: (a) blending a first polymeric material with an antioxidant and a cross-linking agent; (b) blending a second polymeric material with an antioxidant and a cross-linking agent; and (c) consolidating the first polymeric material and the second polymeric material together thereby forming a consolidated, antioxidant and cross-linking agent-blended polymeric material.

In another aspect, the invention provides a method of making an oxidation resistant, cross-linked polymeric material implant. The method includes the steps of: blending a first polymeric material with an antioxidant and a cross-linking agent; (b) blending a second polymeric material with an antioxidant and a cross-linking agent; and (c) consolidating the first polymeric material and the second polymeric material together thereby forming a consolidated, antioxidant and cross-linking agent-blended polymeric material implant.

The method can further include the step of further heating the consolidated, antioxidant and cross-linking agent-blended polymeric material or the implant. The method can further include the step of consolidating the first and the second polymeric material in layers.

In the method, the antioxidant(s) and the cross-linking agents(s) in the first and second polymeric material can be the same. In the method, one or more of the antioxidant(s) and the cross-linking agents(s) in the first and second polymeric material are different.

In another aspect, the invention provides a method of making an oxidation resistant, cross-linked polymeric material. The method includes the steps of: (a) blending a first polymeric material with an antioxidant and a peroxide; (b) blending a second polymeric material with an antioxidant and a peroxide; and (c) consolidating the first polymeric material and the second polymeric material together thereby forming the consolidated, antioxidant and peroxide-blended polymeric material.

In another aspect, the invention provides a method of making an oxidation resistant, cross-linked polymeric material implant. The method includes the steps of: (a) blending a first polymeric material with an antioxidant and a peroxide; (b) blending a second polymeric material with an antioxidant and a peroxide; and (c) consolidating the first polymeric material and the second polymeric material together thereby forming the consolidated, antioxidant and peroxide-blended polymeric material implant.

In the method, the consolidated, antioxidant and peroxide-blended polymeric material can be further heated. In the method, the consolidated, antioxidant and peroxide-blended polymeric material implant can be further heated. The method can further include the step of consolidating the first and the second polymeric material in layers. In the method, the antioxidant(s) and the peroxide(s) in the first and second polymeric material can be the same. In the method, one or more of the antioxidant(s) and the peroxide(s) in the first and second polymeric material can be different. In the method, the heating can be done to a temperature T at about or above (i) a temperature T₁ at which one-half of a quantity of the peroxide decomposes in one hour, or (ii) a temperature T₁₀ at which one-half of a quantity of the peroxide decomposes in ten hours.

The method can further include the step of compression molding the first polymeric material and the second polymeric material below the temperature (T₁ or T₁₀) thereby forming the consolidated, antioxidant and peroxide-blended polymeric material. The consolidating and the heating can be done concurrently. The method can further include the step of machining the oxidation resistant, cross-linked polymeric material into a medical implant. The method can further include the step of packaging and sterilizing the medical implant. The sterilizing can be done by gas sterilization or ionizing irradiation. The method can further include the step of extraction of the oxidation resistant, cross-linked polymeric material. The extraction can be performed by contacting the oxidation resistant, cross-linked polymeric material with a gas, liquid, supercritical fluid, a solid, a solution, an emulsion, or a mixtures thereof. The oxidation resistant, cross-linked polymeric material can be heated during extraction.

In the method, the first polymeric material and the second polymeric material can be selected from ultrahigh molecular weight polyethylenes and mixtures and blends thereof. In the method, the first polymeric material and the second polymeric material can be blended with multiple antioxidants and/or multiple cross-linking agents.

In the method, the peroxide can be selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

The method can include the step of blending the first polymeric material with the antioxidant such that the antioxidant is present in the first polymeric material at a concentration of from 0.001 to 50 wt % by weight of the first polymeric material, or at a concentration of from 0.1 to 2 wt % by weight of the first polymeric material, or at a concentration of from 0.5 to 1 wt % by weight of the first polymeric material, or at a concentration of from 0.6 to 1 wt % by weight of the first polymeric material.

The method can include the step of blending the first polymeric material with the peroxide such that the peroxide is present in the first polymeric material at a concentration of from 0.01 to 50 wt % by weight of the first polymeric material, or at a concentration of from 0.5 to 5 wt % by weight of the first polymeric material.

The method can include the step of compression molding at least one of the first polymeric material and the second polymeric material to a second surface, thereby making an interlocked hybrid material. The second surface can be porous. The second surface can be a porous metal. The method can further include the step of machining the polymeric material or the implant before or after heating.

In another aspect, the invention provides a method of making an oxidation resistant, cross-linked polymeric material. The method includes the steps of: consolidating a polymeric material thereby forming a consolidated polymeric material; and (b) diffusing at least one of (i) an antioxidant and (ii) a crosslinking agent into the consolidated polymeric material.

In another aspect, the invention provides a method of making an oxidation resistant, cross-linked polymeric material implant. The method includes the steps of: consolidating a polymeric material thereby forming a consolidated polymeric material; and (b) diffusing at least one of (i) an antioxidant and (ii) a crosslinking agent into the consolidated polymeric material.

In the method, the antioxidant and cross-linking agent-diffused consolidated polymeric material can be further heated. In the method, the antioxidant and cross-linking agent-diffused consolidated polymeric material implant can be further heated.

In the method, the heating can be to a temperature T at about or above (i) a temperature T₁ at which one-half of a quantity of the crosslinking agent decomposes in one hour, or (ii) a temperature T₁₀ at which one-half of a quantity of the crosslinking agent decomposes in ten hours.

In another aspect, the invention provides a method of making an oxidation resistant, cross-linked polymeric material. The method includes the steps of: (a) consolidating a polymeric material thereby forming a consolidated polymeric material; and (b) diffusing at least one of (i) an antioxidant and (ii) a peroxide into the consolidated polymeric material, thereby forming an antioxidant and peroxide-diffused consolidated polymeric material. In the method, the antioxidant and peroxide-diffused consolidated material can be further heated.

In another aspect, the invention provides a method of making an oxidation resistant, cross-linked polymeric material implant. The method includes the steps of:

(a) consolidating a polymeric material thereby forming a consolidated polymeric material; and (b) diffusing at least one of (i) an antioxidant and (ii) a peroxide into the consolidated polymeric material. In the method, the antioxidant and peroxide-diffused consolidated material implant can be further heated.

In the method, the heating can be to a temperature Tat about or above (i) a temperature T₁ at which one-half of a quantity of the peroxide decomposes in one hour, or (ii) a temperature T₁₀ at which one-half of a quantity of the peroxide decomposes in ten hours.

The method can further include the steps of consolidating the polymeric material with the peroxide; and diffusing the antioxidant into the consolidated polymeric material.

The method can further include the step of consolidating the polymeric material with the peroxide such that the peroxide is present in the polymeric material at a concentration of from 0.01 to 50 wt % by weight of the polymeric material, or at a concentration of from 0.5 to 5 wt % by weight of the polymeric material.

The method can further include the step of heating the antioxidant and peroxide-diffused consolidated polymeric material to a temperature of about 130° C. or above. The method can further include the step of heating the antioxidant and peroxide-diffused consolidated polymeric material to a temperature of about 180° C. or above. The method can further include the step of heating the antioxidant and peroxide-diffused consolidated polymeric material to a temperature of about 300° C. or above.

The method can further include the steps of consolidating the polymeric material with one of (i) the antioxidant and (ii) the peroxide; and diffusing the other of (i) the antioxidant and (ii) the peroxide into the consolidated polymeric material.

The method can further include the step of machining the consolidated polymeric material into a medical implant. In the method, the consolidated polymeric material can be machined into a medical implant or medical implant preform after consolidating the polymeric material.

In the method, the diffusion can be performed at a temperature T at about or above (i) a temperature T₁ at which one-half of a quantity of the peroxide decomposes in one hour, or (ii) a temperature T₁₀ at which one-half of a quantity of the peroxide decomposes in ten hours. In the method, the diffusion can be performed at a temperature T at about or below (i) a temperature T₁ at which one-half of a quantity of the peroxide decomposes in one hour, or (ii) a temperature T₁₀ at which one-half of a quantity of the peroxide decomposes in ten hours. In the method, the diffusion can be performed at a temperature between room temperature and 100° C. In the method, the diffusion can be performed at 100° C. or above.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

In the method, the crosslinking agent is selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof.

In another aspect, the invention provides a method of making an oxidation resistant, cross-linked polymeric material. The method includes the steps of: (a) blending a polymeric material with an antioxidant; (b) consolidating the polymeric material thereby forming a consolidated polymeric material; and (c) diffusing a crosslinking agent into the consolidated polymeric material thereby forming an oxidation resistant, cross-linked polymeric material. In the method, the crosslinking agent-diffused consolidated polymeric material can be further heated.

In another aspect, the invention provides a method of making an oxidation resistant, cross-linked polymeric material implant. The method includes the steps of: (a) blending a polymeric material with an antioxidant; (b) consolidating the polymeric material thereby forming a consolidated polymeric material; and (c) diffusing a crosslinking agent into the consolidated polymeric material. In the method, the consolidated polymeric material can be machined into a medical implant or medical implant preform before diffusing. In the method, the antioxidant-blended polymeric material can be compression molded into implant shape before diffusing.

In the method, the antioxidant-blended polymeric material can be compression molded onto a second material, thereby forming a interlocked hybrid material before diffusing. In the method, the second material can be porous. The second material can be a porous metal.

The method can include the step of blending the polymeric material with the antioxidant such that the antioxidant is present in the polymeric material at a concentration of from 0.001 to 50 wt % by weight of the polymeric material, or at a concentration of from 0.1 to 2 wt % by weight of the polymeric material, or at a concentration of from 0.5 to 1 wt % by weight of the polymeric material, or at a concentration of from 0.6 to 1 wt % by weight of the polymeric material.

In the method, the crosslinking agent can be selected from peroxides and mixtures thereof. In the method, the crosslinking agent can be selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof.

The method can include the step of diffusing the crosslinking agent into the antioxidant-blended, consolidated polymeric material below a temperature T that is selected from (i) a temperature T₁ at which one-half of a quantity of the peroxide decomposes in one hour, or (ii) a temperature T₁₀ at which one-half of a quantity of the peroxide decomposes in ten hours. The method can include the step of diffusing the crosslinking agent into the preform above a temperature T selected from (i) a temperature T₁ at which one-half of a quantity of the peroxide decomposes in one hour, or (ii) a temperature T₁₀ at which one-half of a quantity of the peroxide decomposes in ten hours.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these, and the crosslinking agent can be selected from dicumyl peroxide, benzoyl peroxide, 2,5-Di(tert-butylperoxy)-2,5-dimethyl-3-hexyne, 3,3,5,7,7-pentamethyl 1,2,4-trioxepane, and mixtures thereof.

In another aspect, the invention provides a method of making an antioxidant and crosslinking agent-diffused polymeric material. The method includes the steps of: (a) consolidating a polymeric material thereby forming a consolidated polymeric material; (b) diffusing at least one of (i) an antioxidant and (ii) a crosslinking agent into the consolidated polymeric material, thereby forming an antioxidant and cross-linking agent-diffused polymeric material; and (c) irradiating the antioxidant and crosslinking agent-diffused consolidated polymeric material. In the method, the antioxidant-diffused and/or cross-linking agent-diffused polymeric material can be further heated.

In another aspect, the invention provides a method of making an antioxidant and crosslinking agent-diffused polymeric material implant. The method includes the steps of: (a) consolidating a polymeric material thereby forming a consolidated polymeric material; (b) diffusing at least one of (i) an antioxidant and (ii) a crosslinking agent into the consolidated polymeric material; and (c) irradiating the antioxidant and crosslinking agent-diffused consolidated polymeric material.

The method can include the step of irradiating the consolidated polymeric material at a radiation dose between about 25 kGy and about 1000 kGy. In the method, the consolidated polymeric material can be irradiated at a temperature between about 20° C. and about 135° C. In the method, the consolidated polymeric material can be irradiated at a temperature about 135° C. or above. The method can include the step of compression molding the polymeric material. Diffusing can be performed before irradiating.

In the method, the polymeric material can be selected from ultrahigh molecular weight polyethylenes and mixtures and blends thereof.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

In the method, the crosslinking agent can be selected from dicumyl peroxide, benzoyl peroxide, 2,5-Di(tert-butylperoxy)-2,5-dimethyl-3-hexyne, 3,3,5,7,7-pentamethyl 1,2,4-trioxepane, and mixtures thereof.

In another aspect, the invention provides a method of making an oxidation and wear resistant polymeric material. The method includes the steps of: (a) blending a polymeric material with one or more antioxidant(s) and one or more crosslinking agent(s); (b) consolidating the blended polymeric material thereby forming a consolidated polymeric material; and (c) irradiating the consolidated polymeric material, thereby forming an oxidation and wear resistant polymeric material. In the method, the antioxidant-diffused and cross-linking agent-diffused polymeric material can be further heated.

In another aspect, the invention provides a method of making an oxidation and wear resistant polymeric material implant. The method includes the steps of: (a) blending a polymeric material with one or more antioxidant(s) and one or more crosslinking agent(s); (b) consolidating the blended polymeric material thereby forming a consolidated polymeric material; and (c) irradiating the consolidated polymeric material, thereby forming an oxidation and wear resistant polymeric material implant. In the method, the antioxidant and cross-linking agent-diffused polymeric material can be further heated.

The method can include the step of irradiating the consolidated polymeric material at a radiation dose between about 25 kGy and about 1000 kGy. In the method, the consolidated polymeric material can be irradiated at a temperature between about 20° C. and about 135° C. In the method, the consolidated polymeric material can be irradiated at a temperature about 135° C. or above.

The method can further include the step of compression molding the polymeric material. Consolidating can be performed before irradiating.

In the method, the polymeric material can be selected from ultrahigh molecular weight polyethylenes and mixtures and blends thereof.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

In the method, the crosslinking agent can be selected from dicumyl peroxide, benzoyl peroxide, 2,5-Di(tert-butylperoxy)-2,5-dimethyl-3-hexyne, 3,3,5,7,7-pentamethyl 1,2,4-trioxepane, and mixtures thereof.

In another aspect, the invention provides a method of making an oxidation resistant, cross-linked polymeric material. The method includes the steps of: (a) blending a first polymeric material with a first antioxidant and a first crosslinking agent; (b) blending a second polymeric material with a second antioxidant and optionally a second crosslinking agent; and (c) consolidating the first polymeric material and the second polymeric material thereby forming a consolidated, antioxidant and crosslinking agent-blended polymeric material having a first region of the first polymeric material and having a second region of the second polymeric material, thereby forming a consolidated antioxidant and crosslinking agent-blended polymeric material. The first polymeric material and the second polymeric material can be the same or different, and the first antioxidant and the second antioxidant can be the same or different, and the first crosslinking agent and the second crosslinking agent can be the same or different, and levels of crosslinking can be different in the first layer and the second layer. In the method, the consolidated antioxidant and cross-linking agent-blended polymeric material can be further heated.

In another aspect, the invention provides a method of making an oxidation resistant, cross-linked polymeric material implant. The method includes the steps of: (a) blending a first polymeric material with a first antioxidant and a first crosslinking agent; (b) blending a second polymeric material with a second antioxidant and optionally a second crosslinking agent; and (c) consolidating the first polymeric material and the second polymeric material thereby forming a consolidated, antioxidant and crosslinking agent-blended polymeric material having a first region of the first polymeric material and having a second region of the second polymeric material thereby forming a consolidated antioxidant and crosslinking agent-blended polymeric material implant. The first polymeric material and the second polymeric material can be the same or different, and the first antioxidant and the second antioxidant can be the same or different, and the first crosslinking agent and the second crosslinking agent can be the same or different, and levels of crosslinking can be different in the first layer and the second layer. In the method, the consolidated antioxidant and cross-linking agent-blended polymeric material can be further heated.

In another aspect, the invention provides a method of making an oxidation resistant, cross-linked polymeric material. The method includes the steps of: (a) blending a first polymeric material with a first antioxidant and a first peroxide; (b) blending a second polymeric material with a second antioxidant and optionally a second peroxide; and (c) consolidating the first polymeric material and the second polymeric material thereby forming a consolidated, antioxidant and peroxide-blended polymeric material having a first region of the first polymeric material and having a second region of the second polymeric material thereby forming a consolidated antioxidant and peroxide-blended polymeric material. In the method, the first polymeric material and the second polymeric material can be the same or different, the first antioxidant and the second antioxidant can be the same or different, the first peroxide and the second peroxide can be the same or different, and levels of crosslinking can be different in the first layer and the second layer. In the method, the consolidated antioxidant and peroxide-blended polymeric material can be further heated.

In another aspect, the invention provides a method of making an oxidation resistant, cross-linked polymeric material implant. The method includes the steps of: (a) blending a first polymeric material with a first antioxidant and a first peroxide; (b) blending a second polymeric material with a second antioxidant and optionally a second peroxide; and (c) consolidating the first polymeric material and the second polymeric material thereby forming a consolidated, antioxidant and peroxide-blended polymeric material having a first region of the first polymeric material and having a second region of the second polymeric material thereby forming a consolidated antioxidant and peroxide-blended polymeric material implant. In the method, the first polymeric material and the second polymeric material can be the same or different, the first antioxidant and the second antioxidant can be the same or different, the first peroxide and the second peroxide can be the same or different, and levels of crosslinking can be different in the first layer and the second layer. In the method, the consolidated antioxidant and peroxide-blended polymeric material implant can be further heated.

In the method, the first crosslinking agent and the second crosslinking agent can be selected from peroxides and mixtures thereof. In the method, the first crosslinking agent and the second crosslinking agent can be selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof.

The method can include the step of compression molding the first and the second polymeric material on a third material, thereby making an interlocked hybrid material. The third material can be porous. The third material can be a porous metal.

In the method, the first antioxidant and the second antioxidant are selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

In the method, the first crosslinking agent and the second crosslinking agent are selected from dicumyl peroxide, benzoyl peroxide, 2,5-Di(tert-butylperoxy)-2,5-dimethyl-3-hexyne, 3,3,5,7,7-pentamethyl 1,2,4-trioxepane, and mixtures thereof.

In the method, the first polymeric material and the second polymeric material can be selected from ultrahigh molecular weight polyethylenes and mixtures and blends thereof.

The method can include the step of blending the second polymeric material with the second antioxidant and the second crosslinking agent.

In another aspect, the invention provides a method of making an oxidation resistant, cross-linked polymeric material. The method includes the steps of: (a) heating a consolidated polymeric material to a temperature above the melting temperature, wherein the polymeric material is blended or doped with at least one antioxidant; and (b) diffusing a cross-linking agent into the consolidated polymeric material, thereby forming a cross-linking agent-diffused polymeric material. In the method, the cross-linking agent-diffused polymeric material can be further heated.

In another aspect, the invention provides a method of making an oxidation resistant, cross-linked polymeric material implant. The method can include the step of (a) heating a consolidated polymeric material to a temperature above the melting temperature, wherein the polymeric material is blended or doped with at least one antioxidant; and (b) diffusing a cross-linking agent into the consolidated polymeric material, thereby forming a cross-linking agent-diffused polymeric material implant. In the method, the cross-linking agent-diffused polymeric material implant can be further heated.

In the method, the consolidated polymeric material can be machined into a medical implant or medical implant preform before diffusing. In the method, the polymeric material can be compression molded into implant shape. In the method, the antioxidant-blended polymeric material can be compression molded onto a second material, thereby forming a interlocked hybrid material before heating. The second material can be porous. The second material can be a porous metal.

In another aspect, the invention provides a method of making an oxidation resistant, cross-linked polymeric material. The method includes the steps of: (a) heating a polymeric material to a temperature above the melting temperature, wherein the polymeric material is blended or doped with at least one antioxidant; and (b) diffusing a peroxide into the consolidated polymeric material with a peroxide thereby forming a peroxide-diffused polymeric material. In the method, the peroxide-diffused polymeric material can be further heated.

In another aspect, the invention provides a method of making an oxidation resistant, cross-linked polymeric material implant. The method includes the steps of: (a) heating a polymeric material to a temperature above the melting temperature, wherein the polymeric material is blended or doped with at least one antioxidant; and (b) diffusing a peroxide into the consolidated polymeric material with a peroxide, thereby forming a peroxide-diffused polymeric material implant. In the method, the peroxide-diffused polymeric material implant can be further heated.

In the method, the consolidated polymeric material can be machined into a medical implant or medical implant preform before diffusing.

In the method, the polymeric material can be compression molded into implant shape. In the method, the antioxidant-blended polymeric material can be compression molded onto a second material, thereby forming a interlocked hybrid material before heating. The second material can be porous. The second material can be a porous metal.

In the method, the heating is performed to a temperature T at about or above (i) a temperature T₁ at which one-half of a quantity of the peroxide decomposes in one hour, or (ii) a temperature T₁₀ at which one-half of a quantity of the peroxide decomposes in ten hours.

In the method, the polymeric material can be selected from ultrahigh molecular weight polyethylenes and mixtures and blends thereof.

In the method, the antioxidant can be selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

In the method, the peroxide can be selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof.

In another aspect, the invention provides a method of making an oxidation resistant, cross-linked polymeric material. The method includes the steps of: (a) blending a polymeric material with a vinyl silane and with one or both of (i) an antioxidant and (ii) a free radical initiator to form a blended polymeric material; and (b) consolidating the blended polymeric material thereby forming a consolidated polymeric material; and (c) contacting the consolidated polymeric material with water thereby forming an oxidation resistant, cross-linked polymeric material.

In another aspect, the invention provides a method of making an oxidation resistant, cross-linked polymeric material implant. The method includes the steps of: (a) blending a polymeric material with a vinyl silane and with one or both of (i) an antioxidant and (ii) a free radical initiator to form a blended polymeric material; (b) consolidating the blended polymeric material thereby forming a consolidated polymeric material; and (c) contacting the consolidated polymeric material with water thereby forming an oxidation resistant, cross-linked polymeric material implant.

The method can further include the step of blending the polymeric material with the vinyl silane and the antioxidant and the free radical initiator. The method can include the steps of blending the polymeric material with the vinyl silane and the antioxidant; and diffusing the free radical initiator into the consolidated polymeric material.

The method can include the steps of blending the polymeric material with the vinyl silane and the free radical initiator; and diffusing the antioxidant into the consolidated polymeric material. The method can include the steps of blending the polymeric material with the vinyl silane and the free radical initiator; and diffusing the antioxidant into the consolidated polymeric material. The method can further include the step of contacting the consolidated polymeric material with water in the presence of a catalyst. The method can further include the step of heating the consolidated polymeric material to obtain a silane-grafted polymeric material. The method can further include the step of diffusing a catalyst into the consolidated polymeric material before contacting the consolidated polymeric material with water.

In the method, the polymeric material is selected from ultrahigh molecular weight polyethylenes and mixtures and blends thereof.

In another aspect, the invention provides a method of making an oxidation resistant, cross-linked polymeric material. The method includes the steps of: (a) blending a polymeric material with a vinyl silane; (b) consolidating the blended polymeric material thereby forming a consolidated polymeric material; (c) irradiating the blended polymeric material or the consolidated polymeric material; and (d) contacting the consolidated polymeric material with water thereby forming an oxidation resistant, cross-linked polymeric material.

In another aspect, the invention provides a method of making an oxidation resistant, cross-linked polymeric material implant. The method includes the steps of: (a) blending a polymeric material with a vinyl silane; (b) consolidating the blended polymeric material thereby forming a consolidated polymeric material; (c) irradiating the blended polymeric material or the consolidated polymeric material; and (d) contacting the consolidated polymeric material with water thereby forming an oxidation resistant, cross-linked polymeric material implant.

The method can include the step of irradiating the blended polymeric material or the consolidated polymeric material uses a radiation dose between about 25 kGy and about 1000 kGy. In the method, the irradiating can be done at a temperature between about 20° C. and about 135° C.

The method can further include the step of diffusing an antioxidant into the consolidated polymeric material or the consolidated polymeric material implant.

The method can further include the step of blending an antioxidant with the polymeric material. The method can include the step of contacting the consolidated polymeric material with water in the presence of a catalyst.

The method can include the step of irradiating the blended polymeric material before consolidating the blended polymeric material.

The method can include the step of irradiating the consolidated polymeric material.

In the method, the polymeric material can be selected from ultrahigh molecular weight polyethylenes and mixtures and blends thereof.

These and other features, aspects, and advantages of the present invention will become better understood upon consideration of the following detailed description, drawings and appended claims.

### BRIEF DESCRIPTION OF THE DRAWINGS

FIG. 1 shows a schematic of vinyl silanes.

FIG. 2 shows some processing schemes for cross-linking polymeric material using (1) antioxidant-blended, consolidated polymeric material followed by peroxide diffusion and disassociation/decomposition; (2) peroxide-blended, consolidated polymeric material followed by antioxidant diffusion and peroxide disassociation/decomposition; and (3) antioxidant-blended, consolidated polymeric material followed by peroxide and antioxidant diffusion and peroxide disassociation/decomposition.

FIG. 3 shows some processing schemes for cross-linking polymeric material using (4) antioxidant-blended, consolidated polymeric material followed by irradiation, peroxide diffusion and disassociation/decomposition; (5) peroxide-blended, consolidated, irradiated polymeric material followed by antioxidant diffusion and peroxide disassociation/decomposition; and (6) antioxidant-blended, consolidated, irradiated polymeric material followed by peroxide and antioxidant diffusion and peroxide disassociation/decomposition.

FIG. 4 is a schematic describing silane cross-linking of polymers.

FIG. 5 is a schematic describing some of the embodiments of the invention for making antioxidant-incorporated silane cross-linked polymeric materials.

FIG. 6 shows antioxidant and peroxide-blended, consolidated UHMWPE pucks containing dicumyl peroxide, benzoyl peroxide, and 2,5-Di(tert-butylperoxy)-2,5-dimethyl-3-hexyne (Luperox® 130).

FIG. 7 is a general schematic of alternative manufacturing methods of vitamin E-blended UHMWPE cross-linked using the addition of cross-linking agents such as peroxides. HIPping in FIG. 7 means hot isostatically pressing.

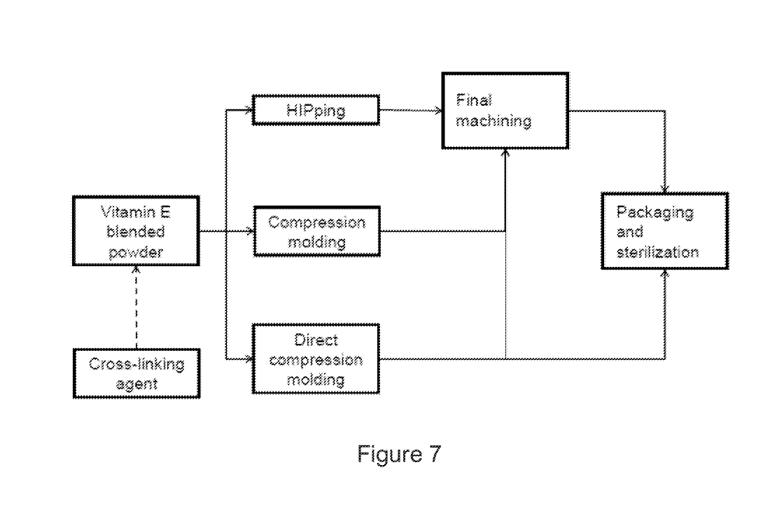

FIG. 8 shows the cross-link density of virgin and vitamin E-blended UHMWPE cross-linked by Luperox®-130 (P130) by blending into powder and decomposing the peroxide during compression molding as a function of peroxide concentration and in comparison to radiation cross-linked (150 kGy) UHMWPE.

FIG. 9 shows a comparison of the radiation dose and peroxide content dependence of the cross-link density of 1 wt % vitamin E-blended UHMWPE cross-linked by radiation (a) and by blending Luperox®-130 (P130) into powder and decomposing the peroxide during compression molding (b).

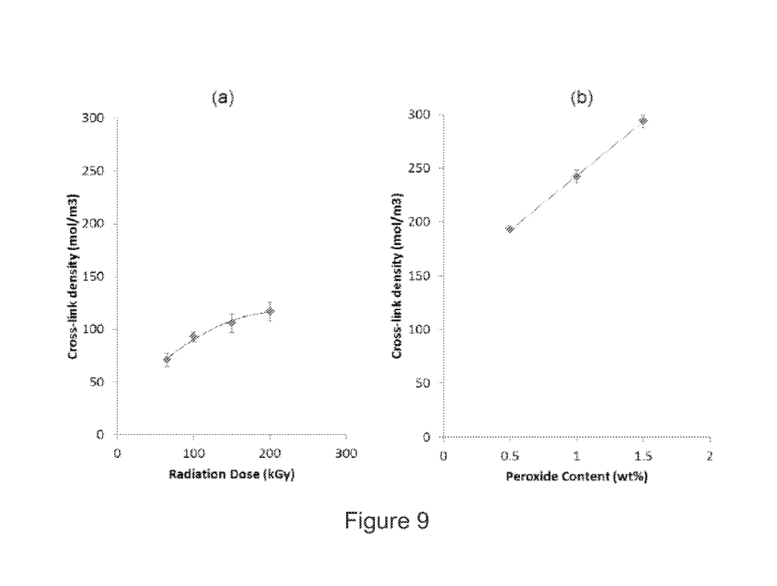

FIG. 10 shows a comparison of the crosslink density dependence of the ultimate tensile strength (a) and elongation-at-break (b) of radiation cross-linked and peroxide cross-linked vitamin E-blended UHMWPE.

FIG. 11 shows the oxidation of virgin and vitamin E-blended UHMWPE cross-linked by 1 wt % Luperox®-130 (P130) during compression molding. Accelerated aging was performed at 70° C. at 5 atm. oxygen for 2 weeks. Thin sections were microtomed and extracted by boiling hexane before analysis.

FIG. 12 shows a comparison of the crosslink density dependence of the crystallinity of radiation cross-linked and peroxide cross-linked vitamin E-blended UHMWPE.

FIG. 13 shows a comparison of the crystallinity dependence of the ultimate tensile strength of radiation cross-linked and peroxide cross-linked vitamin E-blended UHMWPE.

FIG. 14 shows a comparison of the ultimate tensile strength and cross-link density of peroxide cross-linked vitamin E-blended UHMWPE based on processing parameters.

FIG. 15 shows a comparison of the strain at break and cross-link density of radiation cross-linked and peroxide cross-linked vitamin E-blended UHMWPE processed under different conditions.

FIG. 16a shows the cross-link density of 0.1 wt % vitamin E-blended UHMWPE cross-linked using Trigonox® 311 at temperatures below and above its T₁.

FIG. 16b the cross-link density variation on the surface and bulk of the consolidated puck.

FIG. 17 shows the cross-link density of 0.1 wt % vitamin E-blended UHMWPE cross-linked using Trigonox 311 molded at different temperatures above and below T₁ and after annealing after molding above T₁.

FIG. 18 shows the ultimate tensile strength (♦) and elongation-at-break (▪) of 0.1 wt % vitamin E-blended UHMWPE cross-linked using Trigonox 311 molded at different temperatures above and below T₁.

FIG. 19 shows the oxidation index profile of virgin and 0.1 wt % vitamin E-blended UHMWPE cross-linked using 1 wt % Trigonox 311 after accelerated aging.

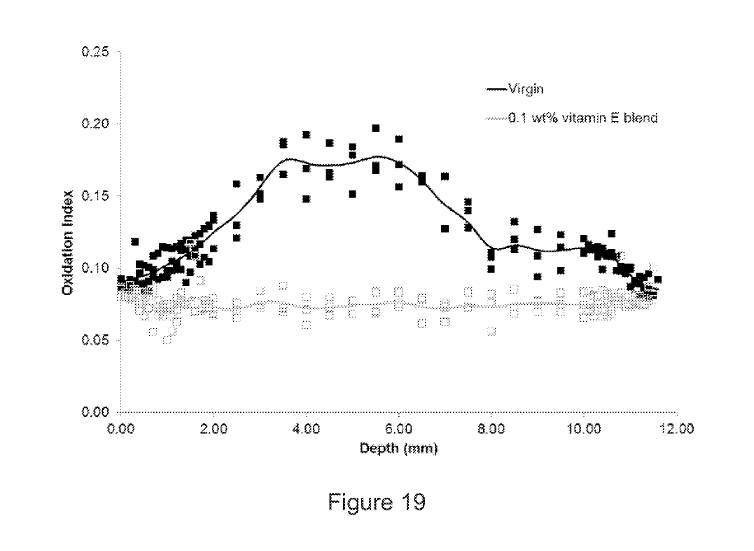

FIG. 20 shows the weight increase due to peroxide and peroxide products after diffusion and decomposition for DCP-doped (a) and P130-doped (b) 0.1 wt % vitamin E-blended UHMWPE as a function of doping temperature. The decomposition temperature was 130° C. for DCP-doped samples and 180° C. for P130-doped samples.

FIG. 21 shows the surface and bulk cross-link density for DCP-doped (a) and P130-doped (b) 0.1 wt % vitamin E-blended UHMWPE as a function of doping temperature. The decomposition temperature was 130° C. for DCP-doped samples and 180° C. for P130-doped samples.

FIG. 22 shows the wear rates for DCP-doped (a) and P130-doped (b) 0.1 wt % vitamin E-blended UHMWPE as a function of doping temperature. The decomposition temperature was 130° C. for DCP-doped samples and 180° C. for P130-doped samples. Control was 0.1 wt % vitamin E-blended UHMWPE without cross-linking.

FIG. 23 shows the cross-link density for DCP-doped (a) and P130-doped (b) 0.1 wt % vitamin E-blended UHMWPE as a function of decomposition temperature. The doping temperature was 80° C. for DCP-doped samples and 100° C. for P130-doped samples.

FIG. 24 shows one method of layered direct compression molding of a peroxide cross-linked UHMWPE with 1 wt % peroxide on the surface and no peroxide in the bulk of the sample. Each layer may contain one or more antioxidants in addition to the cross-linking agent(s).

FIG. 25 shows processing schemes for cross-linking polymeric material using peroxide and radiation cross-linking of antioxidant-containing, consolidated polymeric material.

### DETAILED DESCRIPTION OF THE INVENTION

The present invention relates to methods of making oxidation resistant, wear resistant polymeric materials that contain antioxidant(s) and cross-linking agent(s). In some embodiments the oxidation resistant, wear resistant polymeric material may not contain any of the cross-linking agent; because the cross-linking agent will be used during prior processing to cross-link the polymeric material. The invention also relates to novel methods of cross-linking the polymeric material by blending into polymeric material and diffusing into consolidated polymeric material the cross-linking agent(s). Methods of preparing polymeric materials with spatial control of cross-linking agent to achieve a spatially varying distribution of cross-linking are also provided.

### DEFINITIONS

Peroxide initiation or decomposition temperature (Tₚ): means the temperature at which the peroxide dissociates/decomposes substantially into free radicals which can initiate other reactions, for example at least 0.1%, more preferably at least 10%, or most preferably at least 50% within 1 hour into the free radical(s) that initiate cross-linking in the polymer. Organic peroxides are commonly characterized by their half-lives, i.e., the time it takes for half of a quantity of given peroxide in a given solution to decompose in 1 hour (T₁) or 10 hours (T₁₀). The peroxide initiation temperature, Tₚ, is used generally interchangeably with decomposition temperature, which may be, for example, 5° C. or 10° C. below or 5° C. or 10° C. above the temperature corresponding to the half-life in 10 hours (T₁₀) or to the half-life in 1 hour (T₁). This difference may be because the presence of the peroxide in the polymer rather than that in solution. Peroxide initiation or decomposition temperature can be in the range from −20° C. to 500° C., preferably from 0° C. to 200° C., more preferably from 30° C. to 190° C. It can be 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., or 320° C.

Peroxides are a group of chemicals with the peroxide functional group. General peroxide categories include inorganic peroxides, organic peroxides, diacyl peroxides, peroxyesters, peroxydicarbonates, dialkyl peroxides, ketone peroxides, peroxyketals, cyclic peroxides, peroxymonocarbonates and hydroperoxides. They contain an easily breakable O—O bond that can dissociate/decompose into free radicals when heated and cause cross-linking in polyolefins; therefore peroxides are referred to as part of a family of “cross-linking agents” in this application. Peroxides in this invention can be selected from any peroxide, for example, benzoyl peroxide, dicumyl peroxide, methyl ethyl ketone peroxide, acetone peroxide, 2,5-Di(tert-butylperoxy)-2,5-dimethyl-3-hexyne (Luperox® 130), 3,3,5,7,7-pentamethyl-1,2,4 trioxepane (Trigonox® 311), etc. or mixtures thereof. Other examples of peroxides are dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane. Other examples of peroxides are members of the Luperox® family supplied by Arkema. Other examples of peroxides are 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate. Other examples of peroxides are members of the Trigonox™ or Perkadox™ family supplied by Akzo Nobel.

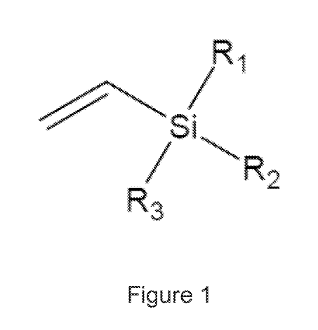

Vinyl silanes are a group of chemicals with a cross-linkable vinyl group to which a silicon atom is attached (Si) to which three other organic groups (R₁, R₂, R₃) can attach (see FIG. 1). In the art, vinyl silane also refers to a vinyl silane with all R groups substituted by hydrogen, but the term “vinyl silane” refers in this document to any member of the vinyl silanes. Some non-limiting examples include vinyltrimethoxysilane, vinyltriethoxysilane, vinyltris(2-methoxyethoxy)silane, and vinyldimethylethoxysilane. For example, R₁, R₂, R₃ can be hydrogen, C₁-C₁₀ substituted or unsubstituted alkyl, or C₁-C₁₀ substituted or unsubstituted alkoxy.

A crosslinking agent is a compound which can cause cross-linking in polymeric materials. Most often, cross-linking of the polymer follows a trigger which initiates the cross-linking process. For example, in the case of peroxides, heating to a temperature where the peroxide decomposes into free radicals, which are then transferred onto the polymer and initiate recombination reactions causing cross-linking is required. In other cases, other stimuli may be used to trigger the reaction such as the application of ultraviolet light, heat, pressure or vacuum, contact with a particular solvent, or irradiation or combinations thereof. In some embodiments, the cross-linking agents used are those that are commercially available and may contain impurities. In some embodiments, the cross-linking agents may be 100% pure or less. In some embodiments, the cross-linking agents are 80%, 85%, 90%, 91%, 92%, 93%, 94%, 95%, 96%, 97%, 98%, or 99% pure.

The definition of crosslinking agent herein differs somewhat from what is known in the art. Typically, a crosslinking agent is defined as a compound which can chemically attach to two or more points on the polymeric material, creating a linkage between the same or different polymer chains. We are using a more general, expanded definition where the crosslinking agent is a compound that initiates the processes that lead to the crosslinking of the polymeric material and the compound may or may not itself chemically or ionically attach to the polymer. For instance, the cross-linking agent may have a free radical, which may abstract a hydrogen from the polymeric material, creating a free radical on the polymeric material; subsequently such free radicals on the polymeric material can react with each other to form a cross-link without chemically attaching the cross-linking agent to the polymeric material. The cross-linking agent may also form covalent or ionic bonding with one or more sites on the polymeric material, thereby causing grafting or cross-linking. In this case, the cross-linking agent becomes part of the cross-linked polymeric material. In some embodiments, there are unreacted cross-linking agent and/or the byproducts of the cross-linking agent in the polymeric material. In some embodiments these unreacted cross-linking agent and/or the byproducts of the cross-linking agent are partially or fully extracted from the polymeric material after cross-linking. This extraction, among other methods, can include solvent extraction, emulsified solvent extraction, heat extraction, supercritical fluid extraction, and/or vacuum extraction. For instance, in some embodiments supercritical carbon dioxide extraction is used. In other embodiments, extraction by placing the polymeric material under vacuum with or without heat is used.

Antioxidants are additives that protect the host polymer against oxidation under various aggressive environments, such as during high temperature consolidation, high temperature crosslinking, low temperature crosslinking, irradiation etc. Some antioxidants act as free radical scavengers in polymeric material during cross-linking. Some antioxidants also act as anti-cross-linking agents in polymeric material during cross-linking; these antioxidants scavenge the free radicals generated on polymeric material during cross-linking, thereby inhibiting or reducing the cross-linking efficiency of the polymeric material. Antioxidants/free radical scavengers/anti-crosslinking agents can be chosen from but not limited to glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these. They can be primary antioxidants with reactive OH or NH groups such as hindered phenols or secondary aromatic amines; they can be secondary antioxidants such as organophosphorus compounds or thiosynergists; they can be multifunctional antioxidants; hydroxylamines; or carbon centered radical scavengers such as lactones or acrylated bis-phenols. The antioxidants can be selected individually or used in any combination. Also, antioxidants can be used with in conjunction with other additives such as hydroperoxide decomposers.

Irganox®, as described herein, refers to a family of antioxidants manufactured by Ciba Specialty Chemicals. Different antioxidants are given numbers following the Irganox® name, such as Irganox® 1010, Irganox® 1035, Irganox® 1076, Irganox® 1098, etc. Irgafos® refers to a family of processing stabilizers manufactured by Ciba Specialty Chemicals. The Irganox® family has been expanded to include blends of different antioxidants with each other and with stabilizers from different families such as the Irgafos® family. These have been given different initials after the Irganox® name, for instance, the Irganox® HP family are synergistic combinations of phenolic antioxidants, secondary phosphate stabilizers and the lactone Irganox® HP-136. Similarly, there are Irganox® B (blends), Irganox® L (aminic), Irganox® E (with vitamin E), Irganox® ML, Irganox® MD families. Herein we discuss these antioxidants and stabilizers by their tradenames, but other chemicals with equivalent chemical structure and activity can be used. In addition, these chemicals can be used individually or in mixtures of any composition. Some of the chemical structures and chemical names of the antioxidants in the Irganox® family are listed in Table 1 below.

TABLE 1
Chemical names and structures of some antioxidants
trademarked under the Irganox ® name.

| Trade- | | |
|---|---|---|
| name | Chemical name | Chemical Structure |
| Irganox ® 1010 | Tetrakis [methylene (3,5-di-tert- butylhydroxy- hydrocinnamate)] methane | 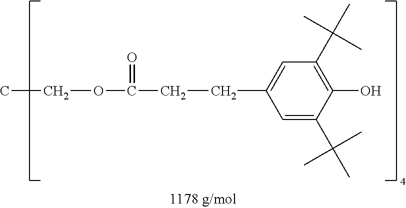 |
| Irganox ® 1035 | Thiodiethylene bis[3-[3,5-di-tert- butyl-4- hydroxyphenyl] propionate] | 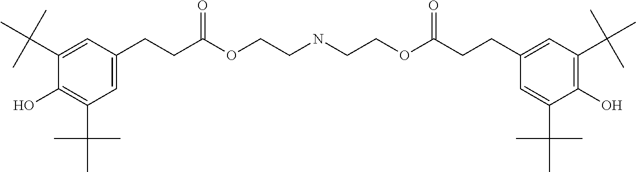 [SMILES: CC(C)(C)c1cc(CCC(=O)OCCNCCOC(=O)CCc2cc(C(C)(C)C)c(O)c(C(C)(C)C)c2)cc(C(C)(C)C)c1O] |
| Irganox ® 1076 | Octadecyl 3,5- di-tert-butyl-4- hydroxyl- hydrocinnamate | 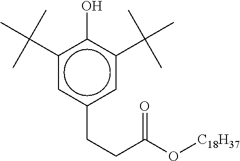 |
| Irganox ® 1098 | N,N′-hexane-1,6- diylbis(3-(3,5-di- tert-butyl-4- hydroxyphenyl- propionamide)) | |
| Irganox ® 1135 | Benzenepropanoic acid, 3,5-bis (1,1-dimethyl- ethyl)-4- hydroxy-C₇- C₉ branched alkyl esters | 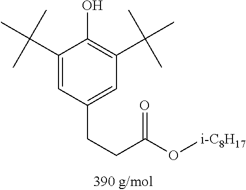 |
| Irganox ® 1330 | 1,3,5-tris(3,5- di-tert-butyl-4- hydroxybenzyl)- 2,4,6- trimethyl- benzene | 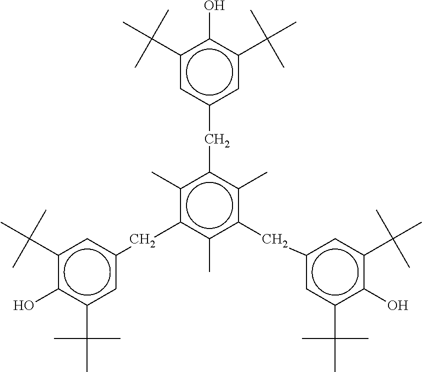 |
| Irganox ® 1520 | | 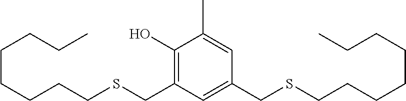 [SMILES: CCCCCCCCSCc1cc(C)c(O)c(CSCCCCCCCC)c1] |
| Irganox ® 1726 | 2,4-bis(dodecyl- thiomethyl)-6- methyl phenol | 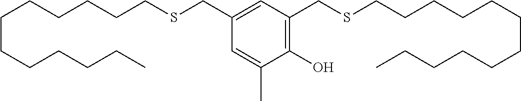 [SMILES: CCCCCCCCCCCCSCc1cc(C)c(O)c(CSCCCCCCCCCCCC)c1] |
| Irganox ® 245 | Triethylene glycol bis(3-tert- butyl-4- hydroxy- 5-methyl- phenyl) propionate | 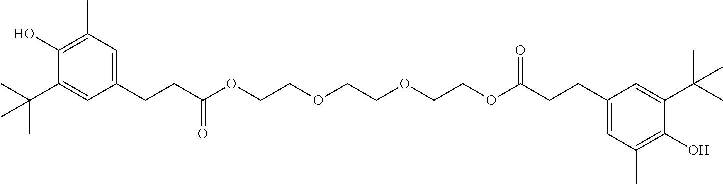 [SMILES: Cc1cc(CCC(=O)OCCOCCOCCOC(=O)CCc2cc(C)c(O)c(C(C)(C)C)c2)cc(C(C)(C)C)c1O] |
| Irganox ® 3052 | 2,2′-methylenebis (4-methyl-6-tert- butylphenol) monoacrylate | 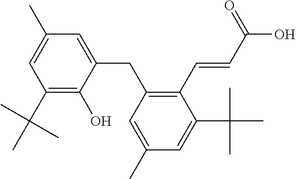 [SMILES: Cc1cc(Cc2cc(C)cc(C(C)(C)C)c2/C=C/C(=O)O)c(O)c(C(C)(C)C)c1] |
| Irganox ® 3114 | 1,3,5-TRis(3,5- di-tert-butyl-4- hydroxybenzyl)- 1,3,5-triazine- 2,4,6(1H,3H, 5H)-trione | 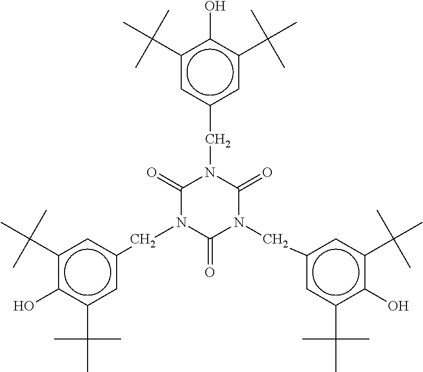 |
| Irganox ® 5057 | Benzenamine,N- phenyl-, reaction products with 2,4,4- trimethylpentene | 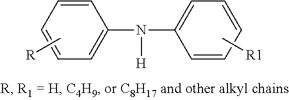 |
| Irganox ® 565 | 2,4-bis(octylthio)- 6-(4-hydroxy-3,5- di-tert- butylanilino)- 1,3,5-triazine | 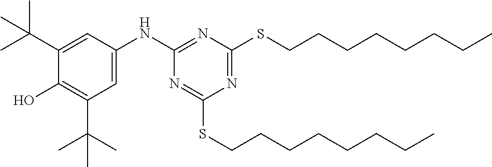 [SMILES: CCCCCCCCSc1nc(Nc2cc(C(C)(C)C)c(O)c(C(C)(C)C)c2)nc(SCCCCCCCC)n1] |
| Irganox ® HP-136 | 5,7-di-t-butyl- 3-(3,4 di- methylphenyl)- 3H-benzofuran- 2-one | 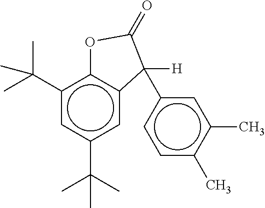 |
| Irgafos ® 168 | Tris(2,4-di-tert- butylphenyl) phospite | 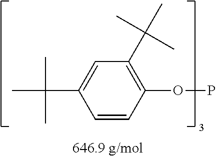 |

Polymeric material: “Polymeric materials” or “polymer” generally refers to what is known in the art as a macromolecule composed of chemically bonded repeating structural subunits. The term “polyethylene article” or “polymeric article” or “polymer” generally refers to articles comprising any “polymeric material” disclosed herein. Polymeric materials include polyethylene, for example, ultrahigh molecular weight polyethylene (UHMWPE). Ultra-high molecular weight polyethylene (UHMWPE) refers to linear substantially non-branched chains of ethylene having molecular weights in excess of about 500,000, preferably above about 1,000,000, and more preferably above about 2,000,000. Often the molecular weights can reach about 8,000,000 or more. By initial average molecular weight is meant the average molecular weight of the UHMWPE starting material, prior to any irradiation. See U.S. Pat. No. 5,879,400, PCT Patent Application Publication No. WO 01/05337, and PCT Patent Application Publication No. WO 97/29793. One example UHMWPE is GUR® ultra-high molecular weight polyethylene available from Ticona. GUR® ultra-high molecular weight polyethylene can be processed by compression molding. Non-limiting examples of UHMWPE are GUR 1050™ and GUR 1020™ available from Ticona.

“Polymeric materials” or “polymers” can also include structural subunits different from each other. Such polymers can be di- or tri- or multiple unit-copolymers, alternating copolymers, star copolymers, brush polymers, grafted copolymers or interpenetrating polymers. They can be essentially solvent-free during processing and use such as thermoplastics or can include a large amount of solvent such as hydrogels. Polymeric materials also include synthetic polymers, natural polymers, blends and mixtures thereof. Polymeric materials also include degradable and non-degradable polymers.

“Polymeric materials” or “polymer” also include such as poly (vinyl alcohol), poly (acrylamide), poly (acrylic acid), poly(ethylene glycol), poly(ethylene oxide), blends thereof, copolymers thereof, or interpenetrating networks thereof, which can absorb water such that water constitutes at least 1 to 10,000% of their original weight, typically 100 wt % of their original weight or 99% or less of their weight after equilibration in water.

“Polymeric material” or “polymer” can be in the form of resin, flakes, powder, consolidated stock, implant, and can contain additives such as antioxidant(s). The “polymeric material” or “polymer” also can be a blend of one or more of different resin, flakes or powder containing different concentrations of an additive such as an antioxidant. The blending of resin, flakes or powder can be achieved by the blending techniques known in the art. The “polymeric material” also can be a consolidated stock of these blends.

“Polymeric material” can be in the form of a consolidated stock that can be machined to form a preform or an implant preform or an implant.

“Polymeric material” can be in the form of a consolidated preform that can be machined to form an implant.

“Polymeric material” can be in the form of a consolidated implant preform that can be machined to form an implant.

“Polymeric material” can be in the form of a direct compression molded implant preform that can be machined to form an implant.

“Polymeric material” can be in the form of a direct compression molded implant preform that can be machined to form an implant.

“Polymeric material” can be in the form of a direct compression molded implant.

What is meant by blend is the combination of two or more constituents to form a mixture thereof. A blend can be formed by the combination of multiple polymers or a combination of additives with one or more types of polymer. For example, an antioxidant/UHMWPE blend may constitute one or more antioxidants mixed with UHMWPE resin powder. The concentration of any of the components or constituents in the blend can be from trace amounts for example 0.0001 wt % to 99.9999 wt %. Typically, an additive will be less than 50% of the blend and the concentration of the polymer or the polymeric material will be more than 50%.

Blending generally refers to mixing of a polymeric material in its pre-consolidated form with an additive. If both constituents are solid, blending can be done by using other component(s) such as a liquid or solvent to mediate the mixing of the two components, after which the liquid is removed by evaporation. If the additive itself is liquid, for example α-tocopherol at room temperature, then the polymeric material can be mixed with large quantities of the liquid additive to obtain a high concentration blend. This high concentration blend can be diluted down to desired blend concentrations with the addition of lower concentration blends or virgin polymeric material without the additive to obtain the desired concentration blend. The high concentration blend and the low concentration blend (or virgin polymeric material without the additive) can be blended together by simple mixing and shaking. This technique of mixing high and low concentration blends also results in improved uniformity of the distribution of the additive in the polymeric material. In the case where an additive is also an antioxidant, for example vitamin E, or α-tocopherol, then blended polymeric material is also antioxidant-doped. Polymeric material, as used herein, also applies to blends of a polyolefin and a cross-linking agent, for example a blend of UHMWPE resin powder blended with peroxide(s) and consolidated. Polymeric material, as used herein, also applies to blends of antioxidant(s), polyolefin(s) and crosslinking agent(s).

In some embodiments the polymeric material is blended with antioxidant(s) first to obtain a polymeric material/antioxidant blend. The said polymeric material/antioxidant blend is then blended with cross-linking agent(s) to obtain a polymeric material/antioxidant/cross-linking agent blend. In other embodiments the order in which the antioxidant(s) and the cross-linking agent(s) are blended together with the polymeric material can be reversed. When multiple antioxidants and cross-linking agents are used, any order of blending step to incorporate the said additives into the polymeric material can be used.

In some embodiments the cross-linking agent(s) and antioxidant(s) are blended together to form a cross-linking agent/antioxidant blend. The said cross-linking agent/antioxidant blend is then blended with polymeric material to obtain a polymeric material/cross-linking agent/antioxidant blend.

What is meant by room temperature is between 15° C. and 30° C.

In one embodiment, UHMWPE flakes are blended with α-tocopherol; preferably the UHMWPE/α-tocopherol blend is heated to diffuse the α-tocopherol into the flakes. This blend is further blended with benzoyl peroxide, dicumyl peroxide, Luperox® 130 (P-130) and/or Trigonox® 311 (T311). This blend is then consolidated. During consolidation, the blend is cross-linked without oxidation.

The products and processes of this invention also apply to various types of polymeric materials, for example, any polypropylene, any polyamide, any polyether ketone, or any polyolefin, including high-density-polyethylene, low-density-polyethylene, linear-low-density-polyethylene, ultra-high molecular weight polyethylene (UHMWPE), copolymers or mixtures thereof. The products and processes of this invention also apply to various types of hydrogel-forming polymers, for example, poly(vinyl alcohol), poly(vinyl acetate), poly(ethylene glycol), poly(ethylene oxide), poly(acrylic acid), poly(methacrylic acid), poly(acrylamide), copolymers or mixtures thereof, or copolymers or mixtures of these with any polyolefin. Polymeric materials, as used herein, also applies to polyethylene of various forms, for example, resin, powder, flakes, particles, powder, or a mixture thereof, or a consolidated form derived from any of the above. Polymeric materials, as used herein, also applies to hydrogels of various forms, for example, film, extrudate, flakes, particles, powder, or a mixture thereof, or a consolidated form derived from any of the above.

The term “additive” refers to any material that can be added to a base polymer or polymeric material in less than 50 v/v %. This material can be an organic or inorganic material with a molecular weight less or more than that of the base polymer or polymeric material. An additive can impart properties to the polymeric material that they polymeric material did not have prior to the addition of the additive, for example, it can be a crosslinking agent that will cross-linked or help cross-linking of the polymeric material or an antioxidant that will improve the oxidative stability of the polymeric material. An additive may be a mixture of antioxidants. In some embodiments an additive may be a mixture of peroxides. In other embodiments an additive may be an antioxidant, a cross-linking agent, a mixture of antioxidants, and mixture of cross-linking agents, a mixture of an antioxidant and a cross-linking agent, or a mixture of antioxidants and cross-linking agents. Additives can also be components that can change the consolidation properties, color properties, processability or can enhance cross-linking properties imparted by cross-linking agent(s).

Doping: Doping refers to a process known in the art (see, for example, U.S. Pat. Nos. 6,448,315 and 5,827,904). In this connection, doping generally refers to contacting a polymeric material with a component or the solution/emulsion of a component under certain conditions, as set forth herein, for example, doping UHMWPE with an antioxidant under supercritical conditions. “Doping” also refers to introducing additive(s) into the base polymeric material in quantities less than 50 v/v %. A polymeric material treated in such a way, for example, to incorporate an antioxidant is termed as an “antioxidant-doped” polymeric material. The polymeric material can be “doped” by other additives as well, such as a crosslinking agent, in which case the polymeric material treated in such a way may be termed as “crosslinking agent-doped” polymeric material. Alternatively, if the polymeric material is doped by one or more peroxides, it may be termed “peroxide-doped” polymeric material.

Doping may also be done by diffusing an additive into the polymeric material by immersing the polymeric material in additive, by contacting the polymeric material with additive in the solid state, by contacting the polymeric material with a bath of additive in the liquid state, or by contacting the polymeric material with a mixture of the additive in one or more solvents in solution, emulsion, suspension, slurry, aerosol form, or in a gas or in a supercritical fluid. The doping process by diffusion can involve contacting a polymeric material, a preform, medical implant or device with an additive, such as 2,5-dimethyl-2,5-Di-(t-butylperoxy)hexyne-3 (Luperox® 130), for about an hour up to several days, preferably for about one hour to 24 hours, more preferably for one hour to 16 hours. The doping time can be from a second to several weeks, or it can be 1 minute to 24 hours, or it can be 15 minutes to 24 hours in 15 minute intervals. The medium for the diffusion of the additive (bath, solution, emulsion, paste, slurry and the like) can be heated to room temperature or up to about 200° C. or more and the doping can be carried out at room temperature or up to about 200° C. or more. Preferably, the antioxidant can be heated to 100° C. and the doping is carried out at 100° C. Or the doping can be carried out at 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 230° C., 240° C., 250° C., 260° C., 270° C., 280° C., 290° C., 300° C., 320° C., or 340° C. If the additive is a peroxide, the doping temperature may be below the peroxide initiation temperature, at the peroxide initiation temperature or above the peroxide initiation temperature or parts of the doping process may be done at different temperatures. A polymeric material incorporated with an additive by diffusion in such a way is termed an “additive-diffused” polymeric material. For example, a polymeric material immersed in a bath of peroxide(s) for enough time to dope at least some parts of the polymeric material with the peroxide, is termed a “peroxide-diffused” polymeric material. The polymeric material can be “diffused” by other additives as well, such as a crosslinking agent, in which case the polymeric material treated in such a way may be termed as “crosslinking agent-diffused”. Alternatively, if the polymeric material is doped by one or more antioxidant(s) by diffusion, it may be termed “antioxidant-diffused”. The polymeric material may be diffused with more than one additive at the same time or at different instances. For example, in such a case where a cross-linking agent and an antioxidant have been introduced into the polymeric material by diffusion, the polymeric material is ‘cross-linking agent and antioxidant-diffused’. The diffusion of additive into polymeric material can be done in any form of the polymeric material, for instance resin, flakes, powder, consolidated form, the form, medical device, finished implant etc. The diffusion or doping of additive into polymeric material can be done by using multiple additives simultaneously.

To increase the depth of diffusion of the antioxidant, the material can be doped for longer durations, at higher temperatures, at higher pressures, and/or in presence of a supercritical fluid.

What is meant by “virgin” is a material with no additives. For instance virgin polymeric material is a polymeric material with no additives such as antioxidants or cross-linking agents.

The doped polymeric material can be annealed (heated) by heating below or above the melting point of the polymeric material subsequent to doping. The annealing is preferably for about an hour up to several days, more preferably for about one hour to 24 hours, most preferably for one hour to 16 hours. The doping time can be from a second to several weeks, or it can be 1 minute to 24 hours, or it can be 15 minutes to 24 hours in 15 minute intervals. The doped polymeric material can be heated to room temperature or up to about 350° C. and the annealing can be carried out at room temperature or up to about 350° C. Preferably, the doped polymeric material can be heated to 120° C. and the annealing is carried out at 120° C. Or annealing can be carried out at 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 315° C., 320° C., 325° C., 330° C., 335° C. or 340° C. In the case of a “peroxide-doped” polymeric material, annealing can cause cross-linking if the temperature(s) used during annealing is close to or above the peroxide initiation temperature(s). Annealing can be performed in liquid(s), in air, in other gases such as oxygen, in inert gas, in supercritical fluid(s), in a sensitizing environment or in vacuum. Annealing can also be performed in ambient pressure, above ambient pressure, or below ambient pressure. Annealing can also be performed while the polymeric material is immersed in liquid antioxidant, such as vitamin E, or a solution/emulsion of antioxidant(s).

A “sensitizing environment” or “sensitizing atmosphere” refers to a mixture of gases and/or liquids (at room temperature) that contain sensitizing gases and/or liquid component(s) that can react with residual free radicals to assist in the recombination of the residual free radicals. The gases maybe acetylene, chloro-trifluoro ethylene (CTFE), ethylene, or like. The gases or the mixtures of gases thereof may contain noble gases such as nitrogen, argon, neon and like. Other gases such as, carbon dioxide or carbon monoxide may also be present in the mixture. In applications where the surface of a treated material is machined away during the device manufacture, the gas blend could also contain oxidizing gases such as oxygen. The sensitizing environment can be dienes with different number of carbons, or mixtures of liquids and/or gases thereof. An example of a sensitizing liquid component is octadiene or other dienes, which can be mixed with other sensitizing liquids and/or non-sensitizing liquids such as a hexane or a heptane. A sensitizing environment can include a sensitizing gas, such as acetylene, ethylene, or a similar gas or mixture of gases, or a sensitizing liquid, for example, a diene. The environment is heated to a temperature ranging from room temperature to a temperature below the melting point of the material.

In certain embodiments of the present invention in which the sensitizing gases and/or liquids or a mixture thereof, inert gas, air, vacuum, and/or a supercritical fluid can be present at any of the method steps disclosed herein, including blending, mixing, consolidating, quenching, irradiating, annealing, mechanically deforming, doping, homogenizing, heating, melting, and packaging of the finished product, such as a medical implant.

The term “free radical initiator” refers to what is known in the art as substances which can yield radical species under certain conditions, for example, by heating. They generally possess bonds that can easily dissociate. For example, peroxide(s) contain easily breakable O—O bonds.

Melting point refers to the peak melting temperature of the polymeric material measured by a differential scanning calorimeter at a heating rate of 10° C. per minute when heating from −100° C. to 400° C. There may be melting of part of the polymeric material at temperatures below this temperature. Typically most semicrystalline polymeric materials start to melt at a temperature lower than the melting point; as the polymeric material is heated more crystals will melt until all crystals are molten.

What is meant by a semi-crystalline polymeric material is a polymeric material that comprises crystalline regions embedded in amorphous regions. In the crystalline domains, some regions of the long molecular polymer chains are aligned to occupy a crystalline lattice forming ordered regions. Crystallization in polymers typically occurs when the polymeric material is being cooled to below its melting point. Depending on the crystallization conditions and the characteristics of the polymer, (such as the composition of the polymer, the melting temperature, the cooling rate, the time in the melt, entanglement density, the molecular weight of the polymer), the crystal lattice and crystal size may change. Due to thermodynamic and kinetic limitations the polymer chains form folds, where the folded regions form the interface between crystal and amorphous regions. In addition, there are some polymeric chain segments spanning between different crystalline regions. In the amorphous regions there is no long range order and the segments of the polymeric material are randomly oriented.

Consolidation refers generally to processes used to convert the polymeric material resin, particles, flakes, i.e., small pieces of polymeric material, into a mechanically integral large-scale solid form, which can be further processed, by for example machining in obtaining articles of use such as preforms, or medical implants. Consolidation methods such as injection molding, extrusion, direct compression molding, compression molding, (cold and/or hot) isostatic pressing, etc. can be used.

In the case of UHMWPE, consolidation is most often performed by “compression molding”. In some instances, consolidation can be interchangeably used with compression molding. The molding process generally involves: (i) heating the polymeric material to be molded, (ii) pressurizing the polymeric material while heated, (iii) keeping at elevated temperature and pressure, and (iv) cooling down and releasing pressure. Typically the consolidation is carried out by pressurizing the heated polymeric material inside a mold to obtain the shape of the said mold with the consolidation of the polymeric material.

In some embodiments, some of the additives or polymeric materials may generate volatile substances during consolidation. In such instances the volatile substances may need to be removed from the mold during consolidation.

Heating and/or pressurizing of the polymeric material during consolidation can be done at any rate. Temperature and/or pressure can be increased linearly with time or in a step-wise fashion or at any other rate. Alternatively, the polymeric material can be placed in a pre-heated environment. The mold for the consolidation can be heated together or separately from the polymeric material to be molded. Steps (i) and (ii), i.e., heating and pressurizing before consolidation can be done in multiple steps and in any order. For example, polymeric material can be pressurized at room temperature to a set pressure level 1, after which it can be heated and pressurized to another pressure level 2, which still may be different from the pressure or pressure(s) in step (iii). Step (iii), where a high temperature and pressure are maintained is the “dwell period” where a major part of the consolidation takes place. One temperature and pressure or several temperatures and pressures can be used during this time without releasing pressure at any point. For example, dwell temperatures in the range of 135° C. to 350° C. and dwell pressures in the range of 0.1 MPa to 100 MPa or up to 1000 MPa can be used. The dwell temperature can be from −20 to 400° C., or can be 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 230° C., 240° C., 250° C., 260° C., 270° C., 280° C., 290° C., 300° C., 320° C. or 340° C. The dwell time can be from 1 minute to 24 hours, more preferably from 2 minutes to 1 hour, most preferably about 10 minutes. For example, dwell time can be 2 hours. Dwell time can be 5, 10, 15, 20, 25, 30, 35, 40, 45, 50, 55, 60 minutes, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9 hours or more. The temperature(s) at step (iii) are termed “dwell” or “molding” temperature(s). The pressure(s) used in step (iii) are termed “dwell” or “molding” pressure(s). In some embodiments, the pressure may increase during the dwell period from the set pressure of the consolidation equipment up to 40 MPa or more. The order of cooling and pressure release step (iv) can be used interchangeably. In some embodiments, the cooling and pressure release may follow varying rates independent of each other. In some embodiments, consolidation of polymeric resin or blends of the resin with crosslinking agent(s) and/or antioxidant(s) are achieved by compression molding. The dwell temperature and dwell time for consolidation can be changed to control the amount of peroxide disassociation/decomposition and therefore desired cross-linking.

In some embodiments, the consolidated polymeric material is fabricated through “direct compression molding” (DCM), which is compression molding using parallel plates or any plate/mold geometry which can directly result in an implant or implant preform. Preforms are generally oversized versions of implants, where some machining of the preform can give the final implant shape.

Compression molding can also be done such that the polymeric material is directly compression molded onto a second surface, for example, a metal or a porous metal to result in an implant or implant preform. This type of molding results in a “hybrid interlocked polymeric material” or “hybrid interlocked material” or “hybrid interlocked medical implant preform” or “hybrid interlocked medical implant” or “monoblock implant”. Molding can be conducted with a metal piece that becomes an integral part of the consolidated polymeric article. For example, a combination of antioxidant-containing polyethylene resin, powder, or flake and virgin polyethylene resin, powder or flake is direct compression molded into a metallic acetabular cup or a tibial base plate. The porous tibial metal base plate is placed in the mold, antioxidant blended polymeric resin, powder, or flake is added on top. Prior to consolidation, the pores of the metal piece can be filled with a waxy or plaster substance through part of the thickness to achieve polyethylene interlocking through the other unfilled half of the metallic piece. The pore filler is maintained through the irradiation and subsequent processing (for example additive diffusion, peroxide and/or antioxidant diffusion) to prevent inextricable infusion of additives in to the pores of the metal. In some embodiments, the article is machined after processing to shape an implant. In some embodiments, there is more than one metal piece integral to the polymeric article. The metal(s) may be porous only in part or non-porous. In another embodiment, one or some or all of the metal pieces integral to the polymeric article is a porous metal piece that allows bone in-growth when implanted into the human body. In one embodiment, the porous metal of the implant is sealed using a sealant to prevent or reduce the infusion of additive such as antioxidant/cross-linking agent (in diffusion steps after consolidation) into the pores during the selective doping of the implant. Preferably, the sealant is water soluble. But other sealants are also used. The final cleaning step that the implant is subjected to also removes the sealant. Alternatively, an additional sealant removal step is used. Such sealants as water, saline, aqueous solutions of water soluble polymers such as poly-vinyl alcohol, water soluble waxes, plaster of Paris, or others are used. In addition, a photoresist like SU-8, or other, may be cured within the pores of the porous metal component. Following processing, the sealant may be removed via an acid etch or a plasma etch.

Compression molding can also be done by “layered molding”. This refers to consolidating a polymeric material by compression molding one or more of its resin forms, which may be in the form of flakes, powder, pellets or the like or consolidated forms in layers such that there are distinct regions in the consolidated form containing different concentrations of additives such as antioxidant(s) or crosslinking agent(s). Whenever a layered-molded polymeric material is described in the examples below and is used in any of the embodiments, it can be fabricated by:
(a) layered molding of polymeric resin powder or its antioxidant/crosslinking agent blends where one or more layers contain no crosslinking agent(s) and one or more layers contain one or more additives, antioxidants and/or crosslinking agents;
(b) molding together of previously molded layers of polymeric material containing different or identical concentration of additives such as antioxidant(s) and crosslinking agent(s) where one or more layers contain no crosslinking agent(s) and one or more layers contain one or more additives, antioxidants and/or anti-crosslinking agents; or
(c) molding of UHMWPE resin powder with or without antioxidant(s) and/or crosslinking agent(s) onto at least one previously molded polymeric material with or without antioxidant(s) and/or crosslinking agent(s) where one or more layers contain no crosslinking agent(s) and one or more layers contain one or more additives, antioxidant(s) and/or crosslinking agent(s).

The layer or layers to be molded can be heated in liquid(s), in water, in air, in inert gas, in supercritical fluid(s) or in any environment containing a mixture of gases, liquids or supercritical fluids before pressurization. The layer or layers can be pressurized individually at room temperature or at an elevated temperature below the melting point or above the melting point before being molded together. The temperature at which the layer or layers are pre-heated can be the same or different from the molding or dwell temperature(s). The temperature can be gradually increased from pre-heat to mold temperature with or without pressure. The pressure to which the layers are exposed before molding can be gradually increased or increased and maintained at the same level.

During molding, different regions of the mold can be heated to different temperatures. The temperature and pressure can be maintained during molding for 1 second up to 1000 hours or longer. During cool-down under pressure, the pressure can be maintained at the molding pressure or increased or decreased. The cooling rate can be 0.0001° C./minute to 120° C./minute or higher. The cooling rate can be different for different regions of the mold. After cooling down to about room temperature, the mold can be kept under pressure for 1 second to 1000 hours. Or the pressure can be released partially or completely at an elevated temperature.

The term “oxidation” refers to the state of polymeric material where reactions with oxygen have taken place such that oxidation products have formed. Generally such a state can be monitored by calculating an ‘oxidation index’ by obtaining a Fourier transform infrared spectrum for the polymeric material after extraction of non-cross-linked components and analyzing the spectrum to calculate an oxidation index, as the ratio of the areas under the 1740 cm⁻¹ carbonyl (limits 1680-1780 cm⁻¹) and 1370 cm⁻¹ (limits 1330-1390 cm⁻¹) methylene stretching absorbance after subtracting the corresponding baselines. Generally speaking an oxidation index of about 0.1 or below is considered baseline levels of oxidation. “Oxidation resistant” refers to a state of polymeric material when there is little or no oxidation or an oxidation index of less than about 0.1 in the material when the material is exposed to oxidizing conditions, for example accelerated aging for 2 weeks at 70° C. in 5 atmospheres of oxygen. “Highly oxidation resistant” refers to a state of polymeric material where there is little or no oxidation or an oxidation index of less than about 0.2 following doping with at least 10 mg of the pro-oxidant squalene diffused into the polymeric material prior to aging and aging for 2 weeks at 70° C. in 5 atmospheres of oxygen.

Crosslinking: Polymeric materials, for example, UHMWPE can be cross-linked by a variety of approaches, including those employing cross-linking chemicals (such as peroxides and/or silane) and/or irradiation. Cross-linked UHMWPE can be obtained according to the teachings of U.S. Pat. Nos. 6,641,617 and 5,879,400, PCT Patent Application Publication Nos. WO 01/05337 and WO 97/29793, and U.S. Patent Application Publication No. 2003/0149125, the entirety of which are hereby incorporated by reference.

The term ‘substantial cross-linked’ refers to the state of a polymeric material where polymer swelling in a good solvent is significantly reduced from the uncross-linked state. For instance, the cross-link density of polyolefins, such as polyethylene is measured by swelling a roughly 3×3×3 mm cube of polymeric material in xylene. The samples are weighed before swelling in xylene at 130° C. for 2 hours and they are weighed immediately after swelling in xylene. The amount of xylene uptake is determined gravimetrically, then converted to volumetric uptake by dividing by the density of xylene; 0.75 g/cc. By assuming the density of polyethylene to be approximately 0.94 g/cc, the volumetric swell ratio of cross-linked UHMWPE is then determined. The cross-link density is calculated by using the swell ratio as described in Oral et al., Biomaterials 31: 7051-7060 (2010) and is reported in mol/m³. The term ‘highly cross-linked’ refers generally to the state of the polymeric material where there is further cross-linking and the cross-link density is higher than that of ‘substantially cross-linked’ polymeric material. The term ‘cross-linked’ refers to the state of polymeric material that is cross-linked to any level, for instance substantial cross-linked or highly cross-linked states.

The term ‘wear’ refers to the removal of material from the polymeric material during articulation or rubbing against another material. For UHMWPE, wear is generally assessed gravimetrically after an initial creep deformation allowance in number of cycles of motion. The term ‘wear resistant’ refers to the state of a polymeric material where it has low wear. For example, the wear rate is tested on cylindrical pins (diameter 9 mm, length 13 mm) on a bidirectional pin-on-disc wear tester in undiluted bovine calf serum at 2 Hz in a rectangular pattern (5 mm×10 mm) under variable load with a maximum of 440 lbs as described in Bragdon et al., J Arthroplasty 16: 658-665 (2001). Initially, the pins are subjected to 0.5 million cycles (MC), after which they are tested to 1.25 million cycles with gravimetric measurements approximately every 0.125 MC. The wear rate is determined by the linear regression of the weight loss as a function of number of cycles from 0.5 to 1.25 MC. The term “highly wear resistant” refers to the state of a polymeric material with a wear rate of less than 3 mg/million-cycles under these conditions.

The term “sterile” refers to what is known in the art; to a condition of an object that is sufficiently free of biological contaminants and is sufficiently sterile to be medically acceptable, i.e., will not cause an infection or require revision surgery. The object, for example a medical implant, can be sterilized using ionizing radiation or gas sterilization techniques. Gamma sterilization is well known in the art. Electron beam sterilization is also used. Ethylene oxide gas sterilization and gas plasma sterilization are also used. Autoclaving is another method of sterilizing medical implants. Exposure to solvents or supercritical fluids for sufficient to kill infection-causing microorganisms and/or their spores can be a method of sterilizing.

The term “heating” refers to the thermal treatment of the polymer at or to a desired heating temperature. In one aspect, heating can be carried out at a rate of about 10° C. per minute to the desired heating temperature. Heating can be carried out at a rate between 0.001° C./min to 1000° C./min, or 0.1° C./min and 100° C./min, or at about 0.2, 0.3, 0.4, 0.5, 0.6, 0.7, 0.8, 0.9, 1° C./min or rates between 1 and 20° C./min in 0.1° C. intervals. In another aspect, the heating can be carried out at the desired heating temperature for a desired period of time. Heating can be performed at a temperature between about −80° C. and about 500° C. or at about 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., or 320° C. In other words, heated polymers can be continued to heat at the desired temperature, below or above the melting point, for a desired period of time. Heating time at or to a desired heating temperature can be at least 1 minute to 48 hours to several weeks long. In one aspect, the heating time is about 1 hour to about 24 hours. For example, the heating is continued for at least for 1 second, 1 minute, 10 minutes, 20 minutes, 30 minutes, one hour, two hours, five hours, ten hours, 24 hours, or more. Or the heating is continued from 10 minutes to 24 hours or more in 10 minute intervals. Cooling after heating can be done at any rate. For example, cooling rate can be about 0.0001° C./min to 1000° C./min, or about 0.1° C./min to 10° C./min, or about 1° C./min or about 2° C./min.

In another aspect, the heating can be carried out for any time period as set forth herein, before or after irradiation. Heating temperature refers to the thermal condition for heating in accordance with the invention. Heating can be performed at any time in a process, including during, before and/or after irradiation. Heating can be done with a heating element. Other sources of energy include the environment and irradiation.

The term “high temperature melting” refers to thermal treatment of the polymer or a starting material to a temperature between about the peak melting temperature of the polymeric material and about 500° C., or 135° C. and about 500° C., or 200° C. and about 500° C. or more, for example, temperature of about 200° C., about 250° C., about 280° C., about 300° C., about 320° C., about 350° C., about 380° C., about 400° C., about 420° C., about 450° C., about 480° C. or more. High temperature melting can be at about 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 315° C., 320° C., 325° C., 330° C., 335° C. or 340° C. Heating time at “high temperature melting” can be at least 30 minutes to 48 hours to several weeks long. In one aspect, the “high temperature melting” time is continued for about 1 minute to about 48 hours or more. For example, the heating is continued for at least for one minute, 10 minutes, 20 minutes, 30 minutes, one hour, two hours, five hours, ten hours, 24 hours, or more. Or the heating is continued from 10 minutes to 24 hours or more in 10 minute intervals. Cooling can be done at any rate. For example, cooling rate can be about 0.0001° C./min to 1000° C./min, or about 0.1° C./min to 10° C./min, or about 1° C./min or about 2° C./min.

The term “annealing” refers to heating or a thermal treatment condition of the polymers in accordance with the invention. Annealing generally refers to continued heating of the polymers at a desired temperature below its peak melting point for a desired period of time, but in the invention refers to the thermal treatment of polymeric material at any desired temperature for a period of time. Annealing can be performed at a temperature between about −80° C. and about 500° C. or at about 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., or 320° C. Annealing time can be at least 1 minute to several weeks long. In one aspect, the annealing time is about 4 hours to about 48 hours, preferably 24 to 48 hours and more preferably about 24 hours. “Annealing temperature” refers to the thermal condition for annealing in accordance with the invention.

The term “packaging” refers to the container or containers in which a medical device is packaged and/or shipped. Packaging can include several levels of materials, including bags, blister packs, heat-shrink packaging, boxes, ampoules, bottles, tubes, trays, or the like or a combination thereof. A single component may be shipped in several individual types of package, for example, the component can be placed in a bag, which in turn is placed in a tray, which in turn is placed in a box. The whole assembly can be sterilized and shipped. The packaging materials include, but are not limited to, vegetable parchments, multi-layer polyethylene, Nylon 6, polyethylene terephthalate (PET), and polyvinyl chloride-vinyl acetate copolymer films, polypropylene, polystyrene, and ethylene-vinyl acetate (EVA) copolymers.

The term “non-permanent device” refers to what is known in the art as a device that is intended for implantation in the body for a period of time shorter than several months. Some non-permanent devices could be in the body for a few seconds to several minutes, while other may be implanted for days, weeks, or up to several months. Non-permanent devices include catheters, tubing, intravenous tubing, and sutures, for example. The term “permanent device” refers to what is known in the art that is intended for implantation in the body for a period longer than several months. Permanent devices include medical devices, for example, acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, and vascular grafts. The term “medical implant” refers to what is known in the art as a device intended for implantation in animals or humans for short or long term use. The medical implants, according to an aspect of the invention, comprises medical devices including acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, and vascular grafts.

The present invention relates generally to methods of making cross-linked, wear and oxidation resistant polymeric materials. Methods of making medical implants containing cross-linked and antioxidant-containing polymers, and materials obtainable thereby, and materials used therewith, also are provided. More specifically, the invention relates to methods of making cross-linked, wear and oxidation resistant antioxidant-containing polymeric materials by using cross-linking agents.

In some embodiments, the cross-linking agent(s) and antioxidant(s) are incorporated with the polymeric material by blending before consolidation of the polymeric material and/or after consolidation. In some embodiments, some cross-linking agent(s) and antioxidant(s) are incorporated before consolidation of the polymeric material and some are incorporated after consolidation.

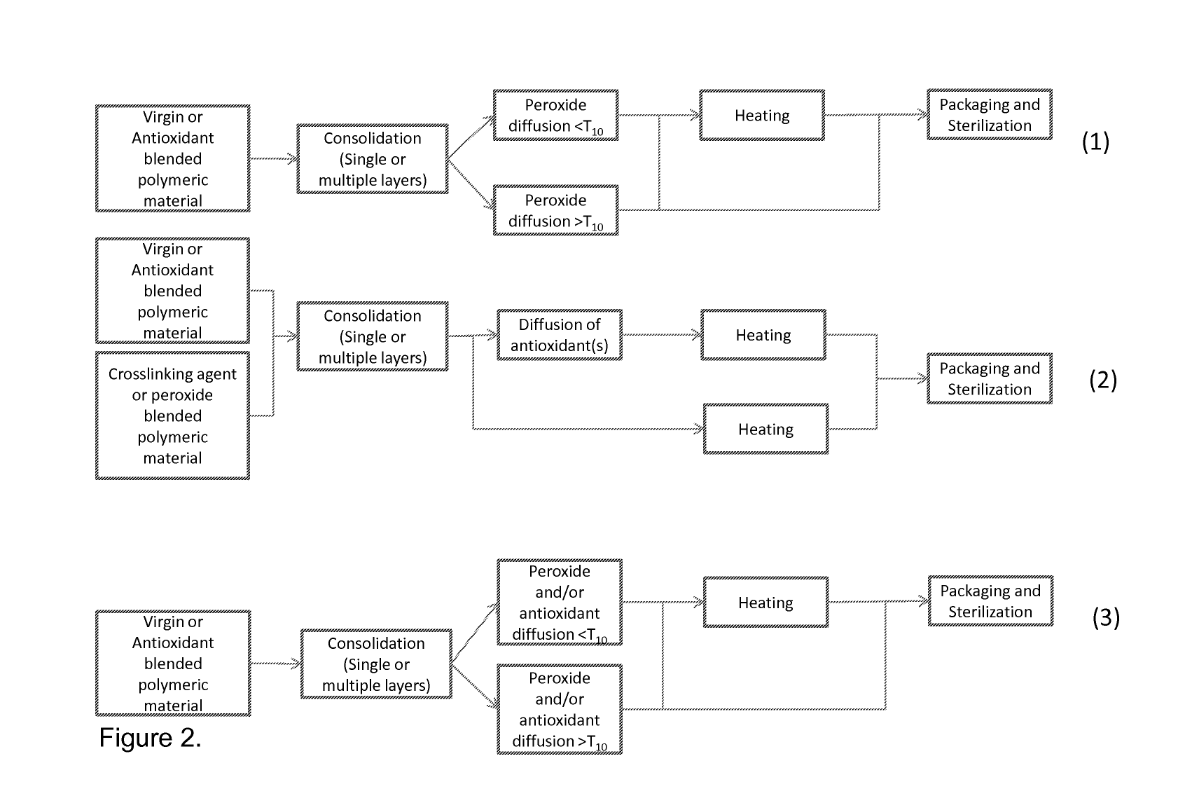

Some non-limiting example embodiments are shown in FIG. 2.

Blending of Antioxidant(s) and Cross-Linking Agent(s) into Polymeric Materials for Cross-Linking

In some embodiments of the invention, one or more antioxidants are used to prevent oxidation in the polymeric materials during manufacturing and in vivo use as medical implants. Such manufacturing methods may include high temperature and pressure such as those commonly used in the consolidation and processing of polymeric materials such as injection molding, compression molding, direct compression molding, screw extrusion, or ram extrusion. In some embodiments of the invention, methods of making medical implant preforms and medical implants are described. Such methods may include machining, packaging and sterilization by radiation and/or gas sterilization methods. Any or all of these methods may initiate oxidation in polymeric materials.

The manufacturing of UHMWPE is commonly performed by compression molding at a temperature between 180° C. and 210° C. in a mold of desired shape in between heated surfaces by bringing the polymeric material resin to dwell or molding temperature (T_{dwell}), pressurizing the polymeric material resin at temperature and maintaining the temperature and pressure (P_{dwell}) for a desired amount of time (t_{dwell}) to affect consolidation of the polymeric material by inter-diffusion of the polymer chains from neighboring resins into each other. The polymeric material resin is cooled under pressure to yield a consolidated polymeric material. Typically, T_{dwell} is between 180° C. and 210° C., t_{dwell} is between 15 minutes and 1 hour, and P_{dwell} is between 10 and 20 MPa. P_{dwell} can be a value between 1 MPa and 100 MPa in 0.5 MPa intervals. In addition, the cooling rate under pressure can contribute to changes in the crystallinity. The cooling rate can be between 0.01° C./min to 200° C./min, preferably 0.5 to 5° C./min, most preferably about 2° C./min. These descriptions also hold true for other polymeric materials where the consolidation temperature is commonly above the glass transition or melting temperature of the polymeric material allowing it to be shaped easily. For example, T_{dwell} can be between −20° C. to 500° C., more preferably 0° C. to 200° C., more preferably from 30° C. to 190° C. Tₚ can be 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., or 190° C. For example, t_{dwell} can be between 1 minute and 24 hours, more preferably 2 minutes to 5 hours, or about 5 minutes or about 2 hours. Or it can be a time from 1 minute to 5 hours in 1 minute intervals. Multiple temperatures and pressures can be used during the dwell cycle.

When the polymeric material is consolidated in the presence of a free radical initiator and/or a cross-linking agent, the consolidation process can lead to oxidation, which degrades the mechanical strength and wear properties of the polymeric material. The presence of antioxidants during consolidation can decrease or eliminate the oxidation caused by the free radical initiators and/or cross-linking agents.

When the polymeric material is consolidated in the presence of peroxide(s), the cross-linking during the consolidation depends on the amount of decomposed peroxide. If T_{dwell} is substantially below the peroxide initiation temperature (Tₚ) or the T₁₀ of the peroxide(s) (whichever one is lower), no substantial cross-linking is expected during the consolidation, which is typically less than 1 hour. If T_{dwell} is between T₁₀ and T₁ of the peroxide, then substantial cross-linking is expected. Therefore, cross-linking of the polymeric material by using peroxides during consolidation can be controlled by the type of peroxide(s), concentration of peroxide(s) and molding factors such as pre-heat temperature, pre-heat time, molding or dwell temperature and molding or dwell time. Typically, T_{dwell} is between 180° C. and 210° C., t_{dwell} is between 15 minutes and 1 hour, and P is between 10 and 20 MPa. P_{dwell} can be a value between 1 MPa and 100 MPa a 0.5 MPa intervals. In addition, the cooling rate under pressure can contribute to changes in the crystallinity. The cooling rate can be between 0.01° C./min to 200° C./min, preferably 0.5 to 5° C./min, most preferably about 2° C./min. These descriptions also hold true for other polymeric materials where the consolidation temperature is commonly above the glass transition or melting temperature of the polymeric material allowing it to be shaped easily. For example, T_{dwell} can be between −20° C. to 500° C., more preferably 0° C. to 200° C., more preferably from 30° C. to 190° C. T_{dwell} can be 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 155° C., 160° C., 165° C., 170° C., 175° C., 180° C., 185° C., or 190° C. For example, t_{dwell} can be between 1 minute and 24 hours, more preferably 2 minutes to 5 hours, or about 5 minutes or about 2 hours. Or it can be a time from 1 minute to 5 hours in 1 minute intervals. The temperature and pressure during consolidation can be increased or decreased stepwise; for example, one temperature and pressure can be maintained for a period of time and then another temperature and pressure can be obtained and maintained during the same molding cycle. For example, the polymeric material can be pre-heated to 190° C. in a mold, then placed in between heated plates at 170° C. and pressurized to 0.1 MPa for 10 minutes, then pressurized to 10 MPa and the pressure and temperature are maintained for 10 minutes, then the temperature can be increased to 180° C. and the pressure can be increased to 20 MPa and the temperature and pressure can be maintained for 10 minutes. Pre-heating before molding is optional. For example, the polymeric material can be placed in a mold at about room temperature and placed in between plates at about 180° C. and pressurized to about 20 MPa and the pressure and temperature maintained for 2 hours. The changes in temperature and pressure can be simultaneous or subsequent to each other. Cooling and heating rates can also be varied.

When the polymeric material is consolidated in the presence of antioxidants, antioxidants can hinder cross-linking in the polymeric material. This effect may be linear or non-linear with increasing concentration. In some embodiments, the total antioxidant concentration in the polymeric material can be from 0.001 to 50 wt %, more preferably 0.1 to 1 wt %, most preferably antioxidant(s) are blended at a concentration of 0.5 wt % or 1 wt %. Antioxidant concentration can be a value between 0.1 and 5 wt % in 0.1 wt % intervals. It can be 0.3, 0.4, 0.5, 0.6, 0.7, 0.8, 0.9 or 1 wt % or more. Antioxidants can be used by themselves or together. They can be blended into the polymeric material in pure form or with the aid of a solvent prior to consolidation. The antioxidants can be incorporated during consolidation or after consolidation. An example of such an antioxidant is vitamin E. Therefore, the cross-linking of the polymeric material by using cross-linking agent(s) in the presence of antioxidants during consolidation can be further controlled by the antioxidant concentration.

When the polymeric material is consolidated in the presence of crosslinking agents, the concentration of the crosslinking agent can be from 0.001 to 50 wt %, more preferably 0.1 to 5 wt %, most preferably the crosslinking agent(s) are blended at a concentration of 0.5 wt % or 1 wt % or 1.5 wt % or 2 wt %. Cross-linking agent concentration can be a value between 0.1 and 5 wt % or 10 wt % in 0.1 wt % intervals. Cross-linking agent can be a peroxide.

In some embodiments of this invention, polymeric material is blended with one or more antioxidants and one or more crosslinking agents. The blend is consolidated into an implant preform. The implant preform is machined to obtain a final implant. The final implant is packaged and sterilized by irradiation or gas sterilization. In some embodiments, one of the antioxidants blended with the polymeric material can be vitamin E.

In one embodiment, UHMWPE is blended with vitamin E and one or more peroxides. The blend is consolidated into an implant preform. The implant preform is machined to obtain a final implant. The final implant is packaged and sterilized by irradiation or gas sterilization.

In one embodiment, UHMWPE is blended with vitamin E and one peroxide. The blend is consolidated into an implant preform. The implant preform is machined to obtain a final implant. The final implant is packaged and sterilized by irradiation or gas sterilization.

In some embodiments, the antioxidant blended into the polymeric material is β-tocopherol. In some embodiments, the concentration of the antioxidant in the antioxidant-blended polymeric material is 0 wt %, 0.2 wt %, or 1 wt %. In some embodiments, the concentration of the peroxide(s) in the polymeric material is 0.05 wt % or 0.1 wt %, or 0.2 wt %, or 0.3 wt %, or 0.4 wt %, or 0.5 wt %, or 0.75 wt %, or 1 wt %, or 2 wt %, or 5 wt % or more.

In one embodiment, polymeric material is blended with one or more peroxides and one or more antioxidants. The blend is consolidated into an implant preform. The peroxides can be chosen such that the initiation temperatures of the peroxides are substantially less than the molding temperature(s). This is such that the consolidated polymeric blend is substantially cross-linked. The cross-linked implant preform is then machined to obtain a final implant. The final implant can be packaged and sterilized by irradiation or gas sterilization.

In one aspect, the invention provides methods of making oxidation resistant, cross-linked polymeric material comprising (a) blending polymeric material with one or more antioxidant(s) and one or more peroxide(s); and (b) consolidating the polymeric material, thereby forming a cross-linked polymeric material.

In one aspect, the invention provides methods of making oxidation resistant, cross-linked medical implant comprising (a) blending polymeric material with one or more antioxidant(s) and one or more peroxide(s); (b) consolidating the polymeric material, thereby forming a cross-linked medical implant preform; and (c) machining the medical implant preform to obtain a medical implant, thereby forming an oxidation resistant, cross-linked medical implant.

In one aspect, the invention provides methods of making a sterile, oxidation resistant, cross-linked medical implant comprising (a) blending polymeric material with one or more antioxidant(s) and one or more peroxide(s); (b) consolidating the polymeric material, thereby forming a cross-linked medical implant preform; (c) machining the medical implant preform to obtain a medical implant, thereby forming an oxidation resistant, cross-linked medical implant; and (d) sterilizing the implant by gas sterilization or radiation sterilization, thereby forming a sterile, oxidation resistant, cross-linked medical implant.

In one aspect, the invention provides methods of making oxidation resistant, cross-linked polymeric material comprising (a) blending polymeric material with one or more antioxidant(s) and one or more peroxide(s); (b) consolidating the polymeric material, thereby forming a consolidated, antioxidant and peroxide-blended polymeric material; and (c) heating the polymeric material, thereby forming a cross-linked consolidated polymeric material.

In some embodiments of this invention, polymeric material is blended with one or more antioxidants and one or more crosslinking agents. The blend is consolidated. The consolidated blend is heated. The consolidated, heated blend is machined to obtain a final implant. The final implant is packaged and sterilized by irradiation or gas sterilization. In some embodiments, one of the antioxidants blended with the polymeric material can be vitamin E. In some embodiments, one or more of the cross-linking agent(s) can be a peroxide.

In one aspect, the invention provides methods of making an oxidation resistant, cross-linked medical implant comprising (a) blending polymeric material with one or more antioxidant(s) and one or more peroxide(s); (b) consolidating the polymeric material, thereby forming a consolidated, antioxidant and peroxide-blended polymeric material; (c) heating the polymeric material, thereby forming a cross-linked consolidated polymeric material; and (d) machining the substantially cross-linked consolidated polymeric material, thereby forming a cross-linked medical implant.

In one aspect, the invention provides methods of making an oxidation resistant, highly cross-linked medical implant comprising (a) blending polymeric material with one or more antioxidant(s) and one or more peroxide(s); (b) consolidating the polymeric material, thereby forming a consolidated, antioxidant and peroxide-blended polymeric material; (c) heating the polymeric material, thereby forming a highly cross-linked consolidated polymeric material; and (d) machining the highly cross-linked consolidated polymeric material, thereby forming a highly cross-linked medical implant.

In one aspect, the invention provides methods of making a sterile, oxidation resistant, cross-linked medical implant comprising (a) blending polymeric material with one or more antioxidant(s) and one or more peroxide(s); (b) consolidating the polymeric material, thereby forming a consolidated, antioxidant and peroxide-blended polymeric material; (c) heating the polymeric material, thereby forming a cross-linked consolidated polymeric material; (d) machining the cross-linked consolidated polymeric material, thereby forming a cross-linked medical implant; and (e) sterilizing the medical implant by gas sterilization and radiation sterilization.

In one aspect, the invention provides methods of making a sterile, oxidation resistant, highly cross-linked medical implant comprising (a) blending polymeric material with one or more antioxidant(s) and one or more peroxide(s); (b) consolidating the polymeric material, thereby forming a consolidated, antioxidant and peroxide-blended polymeric material; (c) heating the polymeric material, thereby forming a highly cross-linked consolidated polymeric material; (d) machining the highly cross-linked consolidated polymeric material, thereby forming a highly cross-linked medical implant; and (e) sterilizing the medical implant by gas sterilization and radiation sterilization.

In one embodiment, polymeric material is blended with one or more peroxide(s) and one or more antioxidant(s). The blend is consolidated into an implant preform. The peroxides can be chosen such that the initiation temperatures of the peroxides are higher than the temperatures used during consolidation. This is such that the consolidated polymeric blend is not cross-linked. The consolidated blend is then heated to above the initiation temperature of the peroxides such that chemical cross-linking is achieved. The implant preform can be machined to obtain a final implant before or after the heating step after consolidation. The final implant can be packaged and sterilized by irradiation or gas sterilization.

In one embodiment, polymeric material is blended with one or more peroxide(s) and one or more antioxidant(s). The blend is consolidated into an implant preform. The peroxides can be chosen such that the initiation temperatures of some of the peroxides are substantially less than the molding temperature. This is such that the consolidated polymeric blend is cross-linked. The consolidated blend can then be heated to above the initiation temperature of all of the peroxides such that further chemical cross-linking is achieved. The implant preform can be machined to obtain a final implant before or after the heating step after consolidation. The final implant can be packaged and sterilized by irradiation or gas sterilization.

The concentration of cross-linking agents blended into the polymeric material can be from 0.001 wt % to 50 wt %, more preferably 0.1 wt % to 10 wt %, more preferably 0.5 wt % to 5 wt %, most preferably about 1 wt %.

It is likely that under “conventional” conditions, i.e., temperatures of 170° C.-210° C. and 10-20 MPa of pressure in the molding step with a dwell time up to 20 minutes, blending virgin UHMWPE (no additives) or antioxidant-blended UHMWPE with traditional peroxides (peroxides with T₁₀<150° C.) will result in substantial cross-linking of the polymeric material in resin form before consolidation can take place; thus limit the mechanical integrity of the consolidated polymeric material. This problem can be solved in two ways; (1) deviating from “conventional” conditions by using novel molding conditions specific to the peroxide(s) used in the blend to prevent substantial cross-linking of the peroxide-blended UHMWPE before consolidation, and (2) use of peroxides with T₁₀>150° C., more preferably closer to the molding temperature such that after consolidation, substantial cross-linking is not achieved. In such a material, cross-linking is then achieved by heating the consolidated polymeric material to temperature(s) at or above T₁₀ for at least 1 hour up to 24 hours or longer.

### Blending Process

If the cross-linking agent(s) and/or antioxidant(s) to be blended with the polymeric material are solid, then they can be dry mixed with the polymer resin manually or by using a mixer. If the polymeric material is not a powder, it can be made into powder by using a pulverizer. Alternatively, if any component is liquid, it can be mixed in pure form directly into the polymeric material. Alternatively the additive can be dissolved in a solvent to form an additive solution. The additive solution can then be mixed with the polymeric material and the solvent can be evaporated thereafter.

In any of the embodiments of this invention, where the cross-linking agent(s) and/or antioxidant(s) are blended with the polymeric material, solvent(s) can be used to aid the dispersion of the components in the subsequently consolidated blend. Any solvent, in which one or more of the components are soluble or dispersed, can be used. In some embodiments, it is preferred that the cross-linking agent(s) and/or antioxidant(s) are soluble in isopropanol, ethanol, or acetone. Different solvents can be used to blend different components simultaneously or in any sequence. After the blending, it is preferred that the solvent(s) are evaporated before consolidation of the blend. In any of the embodiments, components can be mixed with each other simultaneously or in any sequence.

In some embodiments, cross-linking of the polymeric material can be achieved before consolidation by blending with one or more cross-linking agent(s) and triggering the reactivity of the cross-linking agent(s). In some embodiments, at least one of the cross-linking agent(s) is a peroxide; the reactivity of peroxide is triggered by decomposing the peroxide with heat. In some embodiments, cross-linking of the polymeric material already containing one or more antioxidant(s) can be achieved by blending with one or more cross-linking agent(s) and triggering the reactivity of the cross-linking agent(s). In some embodiments, cross-linking of the polymeric material can be achieved before consolidation by blending with one or more peroxide(s) and triggering the decomposition of the peroxides by heating the polymeric material blended with peroxide(s) to above the initiation temperature of at least one of the peroxides for a period of time to allow substantial cross-linking. This time can be between 30 seconds and 24 hours or longer, more preferably between 2 minutes and 30 minutes, more preferably between 5 and 20 minutes. Or it can be 2 hours. It can be from 1 hour to 36 hours in 30 minute intervals. In some embodiments, this cross-linked blend of polymeric material with cross-linking agent(s) and/or antioxidant(s) can be consolidated into an implant preform. Further cross-linking can occur during consolidation and after consolidation. The implant preform can be machined to obtain a final implant before or after the heating step after consolidation. Alternatively, direct compression molding can be used to obtain a medical implant after consolidation. The final implant can be packaged and sterilized by irradiation or gas sterilization.

In one embodiment, an antioxidant, for example vitamin E, is dissolved in isopropyl alcohol. The solvent with the antioxidant is mixed with the polymeric material resin, flakes or powder. The isopropyl alcohol is evaporated to obtain an antioxidant-blended polymeric material. A cross-linking agent, for example a peroxide, is dissolved in isopropyl alcohol. The solvent with the cross-linking agent is mixed with the antioxidant-blended polymeric material. The isopropyl alcohol is evaporated to obtain a cross-linking agent and antioxidant-blended polymeric material. More than one antioxidant or more than one cross-linking agent can be mixed in the blend in this manner simultaneously or in any sequence.

In one embodiment, one or more antioxidant(s) and one or more cross-linking agent(s) are mixed with polymeric material in dry form or with the aid of a solvent. For example, vitamin E is dissolved in isopropyl alcohol and mixed with UHMWPE resin powder such that the vitamin E concentration in UHMWPE is 0.1 wt %. The mixture is dried. Then, the vitamin E-blended UHMWPE is mixed with an isopropyl alcohol solution of dicumyl peroxide. The mixture is dried, obtaining a vitamin E and dicumyl peroxide-blended UHMWPE powder. Then, the blend can be consolidated into an implant preform. Alternatively the implant preform can be heated to complete the decomposition of the peroxide if it was not complete during the consolidation step. The implant preform can be machined into a final implant. The final implant can be packaged, and sterilized by irradiation or gas sterilization.

In some embodiments, the cross-linking agent in the polymeric material is a peroxide. During consolidation of the polymeric material if the peroxide decomposition is not complete the consolidated polymeric material is heated and/or annealed to further the peroxide decomposition. This furtherance may be to near complete decomposition or less.

In some embodiments, further treatments after consolidation of blends can be performed. For example, the consolidated blend can be annealed at a temperature below or above the melting temperature of the polymeric material. The annealing temperature can be between −20° C. to 500° C., more preferably 0° C. to 200° C., more preferably from 30° C. to 190° C. The annealing temperature can be 40° C., 120° C., 130° C., 135° C., 140° C., 150° C., 170° C., 200° C. or 300° C. Heat treatment at any step before, during or after consolidation can be performed in air, in inert gas, in supercritical fluids, in vacuum or in sensitizing gas, such as acetylene.

In some embodiments, the methods described by Gul (see, “The effects of peroxide content on the wear behavior, microstructure and mechanical properties of peroxide crosslinked ultra-high molecular weight polyethylene used in total hip replacement”, Journal of Materials Science: Materials in Medicine, vol. 19, issue 6, pages 2427-2435 (2008)) are used to incorporate peroxides to antioxidant-blended or antioxidant-diffused polymeric material, for example UHMWPE.

In some embodiments, the polymeric material, consolidated polymeric material, preform, implant preform, implant, medical device that was cross-linked using a cross-linking agent can be subjected to processes to extract unreacted cross-linking agent, byproducts of the chemical cross-linking, and/or other low molecular weight species resulting from previous processing steps. Typically this extraction may be through heating, applying vacuum, submerging in solvents, and/or such methods.

In a preferred embodiment, ultrahigh molecular weight polyethylene resin powder is blended with vitamin E and 2,5-dimethyl-2,5-Di-(t-butylperoxy)hexyne-3. The blend is consolidated using compression molding, direct compression molding, hot isostatic pressing or ram extrusion such that substantial cross-linking takes place during consolidation. The consolidation temperature can be 170° C., 180° C., 190° C., 200° C., 210° C., 220° C. or more. The dwell time at temperature and pressure can be 2 minutes to 24 hours or more. More preferably, the dwell time at temperature and pressure is about 2 hours. The vitamin E concentration can be 0.5 wt %, 0.6 wt %, 0.8 wt % or 1 wt % or more. The peroxide concentration can be 0.5 wt %, 1 wt %, 1.1 wt %, 1.2 wt %, 1.3 wt %, 1.4 wt %, or 1.5 wt % or more. Then, the consolidated and cross-linked antioxidant-blended polymeric material is heated for a period of time, then cooled. Heating can be done to 130° C., 150° C., 170° C., 190° C., 200° C., 210° C., 220° C., 230° C. or more. Then, the cross-linked antioxidant-blended polymeric material is machined into final implant shape. The implant is packaged and sterilized. Sterilization is done by a gas sterilization method or by ionizing radiation.

In a preferred embodiment, ultrahigh molecular weight polyethylene resin powder is blended with vitamin E and 2,5-dimethyl-2,5-Di-(t-butylperoxy)hexyne-3. The blend is consolidated using direct compression molding onto a porous metal surface such that substantial cross-linking takes place during consolidation. The consolidation temperature can be 170° C., 180° C., 190° C., 200° C., 210° C., 220° C. or more. The dwell time at temperature and pressure can be 2 minutes to 24 hours or more. More preferably, the dwell time at temperature and pressure is about 2 hours. The vitamin E concentration can be 0.5 wt %, 0.6 wt %, 0.8 wt % or 1 wt % or more. The peroxide concentration can be 0.5 wt %, 1 wt %, 1.1 wt %, 1.2 wt %, 1.3 wt %, 1.4 wt %, or 1.5 wt % or more. The consolidation of the polymeric material onto the porous metal creates an interlocked hybrid material. Then, the consolidated and cross-linked antioxidant-blended polymeric material is heated for a period of time, then cooled. Heating can be done to 130° C., 150° C., 170° C., 190° C., 200° C., 210° C., 220° C., 230° C. or more. Then, the interlocked hybrid material is machined into final implant shape. The implant is packaged and sterilized. Sterilization is done by a gas sterilization method or by ionizing radiation.

In a most preferred embodiment, ultrahigh molecular weight polyethylene resin powder is blended with vitamin E and 2,5-dimethyl-2,5-Di-(t-butylperoxy)hexyne-3. The blend is consolidated using compression molding, direct compression molding, hot isostatic pressing or ram extrusion such that substantial cross-linking takes place during consolidation. The consolidation temperature can be 170° C., 180° C., 190° C., 200° C., 210° C., 220° C. or more. The vitamin E concentration can be 0.5 wt %, 0.6 wt %, 0.8 wt % or 1 wt % or more. The peroxide concentration can be 0.5 wt %, 1 wt %, 1.1 wt %, 1.2 wt %, 1.3 wt %, 1.4 wt %, or 1.5 wt % or more. Then, the consolidated and cross-linked antioxidant-blended polymeric material is machined into final implant shape. The implant is packaged and sterilized. Sterilization is done by a gas sterilization method or by ionizing radiation.

In a most preferred embodiment, ultrahigh molecular weight polyethylene resin powder is blended with vitamin E and 2,5-dimethyl-2,5-Di-(t-butylperoxy)hexyne-3. The blend is consolidated using direct compression molding onto a porous metal such that substantial cross-linking takes place during consolidation. The consolidation temperature can be 170° C., 180° C., 190° C., 200° C., 210° C., 220° C. or more. The vitamin E concentration can be 0.5 wt %, 0.6 wt %, 0.8 wt % or 1 wt % or more. The peroxide concentration can be 0.5 wt %, 1 wt %, 1.1 wt %, 1.2 wt %, 1.3 wt %, 1.4 wt %, or 1.5 wt % or more. The consolidation of the polymeric material onto the porous metal creates an interlocked hybrid material. Then, the consolidated and cross-linked antioxidant-blended polymeric material is machined into final implant shape. The implant is packaged and sterilized. Sterilization is done by a gas sterilization method or by ionizing radiation.

### Gradients

In some embodiments, it is desirable to have preferential cross-linking in parts of the consolidated polymeric material or medical implant preform or medical implant. Spatial control of cross-linking methods using control of antioxidant concentrations and irradiation have been described in PCT Patent Application Publication No. WO 2008/092047 to Muratoglu et al. and PCT Patent Application Publication No. WO 2010/096771 to Oral et al., which are incorporated herein by reference. In some applications, it is desirable to have cross-linking limited to a surface layer or a skin layer on the surface of the polymeric material, preform, or implants where cross-linking can be used to improve wear resistance while the remaining regions are not cross-linked at all or are not as highly cross-linked as the surface.

In some embodiments, the polymeric material blended with peroxides and/or antioxidants can have a uniform concentration of the additives after consolidation. In some embodiments, some part(s), for example, surfaces of the consolidated polymeric material can have different concentrations of one or more additives than other part(s), for example the bulk of the consolidated polymeric material. The surface of the consolidated polymeric material or medical implant preform can be about 300 micrometers to about 5 centimeters, more preferably about 1 to 5 millimeters, 2 to 4 millimeters, or 2 millimeters.

In some embodiments, the polymeric material is blended with antioxidant(s) and cross-linking agent(s). At least one cross-linking agent can be a peroxide. The polymeric material is consolidated in layers where one layer contains more cross-linking agent than others. Cross-linking agent is triggered during consolidation such that a cross-linked consolidated polymeric material is obtained after consolidation. In the case of the peroxide(s), consolidation is performed close to or above the decomposition temperature of the peroxide such that cross-linking takes place during consolidation. An antioxidant-containing consolidated polymeric material with spatial control of cross-linking is achieved with regions with high amounts of cross-linking agent resulting in higher cross-link density. Then, the polymeric material can be machined into an implant. The implant can be packaged and sterilized.

Alternatively, in some embodiments, polymeric material is blended with antioxidant(s) and cross-linking agent(s). At least one cross-linking agent can be a peroxide. The polymeric material is consolidated in layers where one layer contains more cross-linking agent than others. A consolidated polymeric material is obtained after consolidation. Then, the consolidated polymeric material is further heated to further cross-link the consolidated polymeric material. In the case of the peroxide(s), consolidation is performed such that some or no cross-linking takes place during consolidation and further cross-linking takes place during heating after consolidation. An antioxidant-containing consolidated polymeric material with spatial control of cross-linking is achieved with regions with high amounts of cross-linking agent resulting in higher cross-link density. Then, the polymeric material can be machined into an implant. The implant can be packaged and sterilized.

In a preferred embodiment, ultrahigh molecular weight polyethylene is blended with vitamin E and 2,5-dimethyl-2,5-Di-(t-butylperoxy)hexyne-3. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide concentration can be 0.1 wt % to 5 wt % or more, preferably 0.5 wt % to about 2 wt %, most preferably about 1 to 1.5 wt %. Then, the peroxide and antioxidant-blend is consolidated into an implant preform by layering a vitamin E-blended UHMWPE blend (without peroxide) and compression molding. The consolidation temperature can be 170° C., 180° C., 190° C., 200° C., 210° C., 220° C. or more. The dwell time at temperature and pressure can be 2 minutes to 24 hours or more. More preferably, the dwell time at temperature and pressure is about 2 hours. Then, consolidated and cross-linked antioxidant-blended polymeric material is machined into an implant. The implant is packaged and sterilized. Sterilization is done by a gas sterilization method or by ionizing radiation.

In some embodiments, the polymeric material is blended with antioxidant(s). Then the antioxidant-blended polymeric material can be machined into an implant or implant preform. Cross-linking agent(s) are diffused into the implant or implant preform. The depth of diffusion can be varied depending on the diffusion parameters. Cross-linking agent can be triggered such that a cross-linked implant or implant preform is obtained. At least one cross-linking agent can be a peroxide. In the case of peroxides, cross-linking can be (further) triggered by heating the implant preform or implant to close to or above the decomposition temperature(s) of the peroxide(s). In some embodiments the temperature of diffusion will be high enough to decompose the peroxide as it diffuses in to the implant or implant preform or the polymeric material, thereby cross-linking the polymer during diffusion. An antioxidant-containing consolidated polymeric material with spatial control of cross-linking is achieved with regions with high amounts of cross-linking agent resulting in higher cross-link density. Then, the polymeric material can be machined into an implant. The implant can be packaged and sterilized.

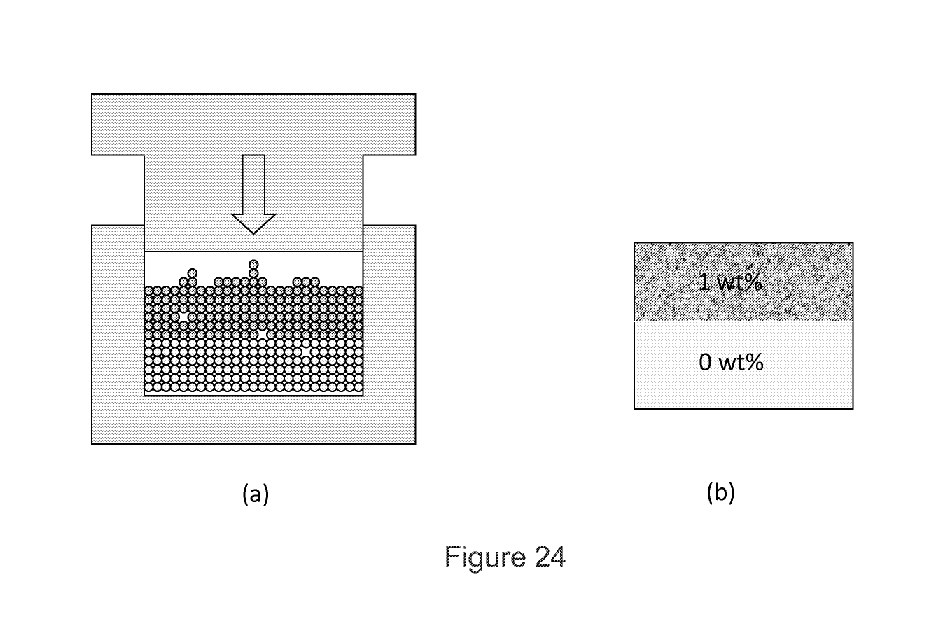

In another embodiment, the consolidated polymeric material with a spatial distribution of cross-links is fabricated through direct compression molding (DCM). The DCM mold is filled with a combination of polyethylene powder containing antioxidant(s) and a high concentration of cross-linking agent and with polyethylene powder containing no or a low concentration of cross-linking agent (see schematic diagram in FIG. 24). The mold is then heated and pressurized to complete the DCM process. The consolidated polymeric material thus formed comprises substantially cross-linked regions. The concentration of cross-linking agent(s) in the cross-linking agent-rich region(s) is between about 0.0005 wt % and about 20 wt % or higher, preferably between 0.005 wt % and 5.0 wt %, preferably about 0.5 wt % or 1.0 wt %. The concentration of the cross-liking agent(s) in the other region(s) is between about 0 wt % and about 20 wt % or higher, preferably about 0 wt % to 0.5 wt %, most preferably about 0 wt % to 0.1 wt %. The antioxidant(s) contained in the different regions can be the same, similar or different concentrations. These concentrations can be between about 0.001 wt % to about 50 wt % or higher, more preferably between about 0.1 wt % and 1.5 wt %, most preferably between about 0.5 wt % to 1 wt %.

In another embodiment, the invention provides methods of making an oxidation-resistant cross-linked polymeric material comprising: a) doping a consolidated polymeric material containing antioxidant(s) with cross-linking agent(s) by diffusion below or above the melting point of the polymeric material, wherein the surface (exterior regions) of the polymeric material contains a higher concentration of cross-linking agent(s) and bulk (generally the interior regions) of the polymeric material contains a lower concentration of cross-linking agent(s), thereby allowing a spatial distribution of the cross-linking agent-rich and cross-linking agent-poor regions; and b) heating the doped polymeric material to close to or above the decomposition temperature of the cross-linking agent, thereby forming an oxidation-resistant cross-linked polymeric material having a spatially controlled antioxidant distribution and/or cross-linking. At least one of the antioxidants can be vitamin E. At least one cross-linking agent can be a peroxide. Heating during diffusion can enable some or all of the cross-linking. Cross-linking can be completed or furthered by the heating step after the diffusion of the cross-linking agents.

In any of the embodiments, irradiation can be used before, during or after cross-linking by the cross-linking agent(s). Radiation can be used for the purposes of cross-linking the material, for grafting components such as antioxidants to the polymeric material or for sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is direct compression molded into final implant shape. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is layered on a second layer of vitamin E blended UHMWPE without any peroxide and is direct compression molded into final implant shape. The final implant can be a tibial insert for total knee arthroplasty. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is direct compression molded into final implant or implant preform shape. The final implant or the preform is heated for a period of time. The heating can be performed at 130, 150, 160, 170, 180° C., 190° C., 200° C., 210° C., 220° C. or more. The heating can be performed for 1, 2, 3, 4 or 5 hours or more. The heated final implant or the implant preform is cooled. If the article is not in its final shape, it can be machined into final implant shape. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is layered on a second layer of vitamin E blended UHMWPE without peroxides and direct compression molded into final implant or implant preform shape. The final implant or implant preform is heated for a period of time. The heating can be performed at 130° C., 150° C., 160° C., 170° C., 180° C., 190° C., 200° C., 210° C., 220° C. or more. The heating can be performed for 1, 2, 3, 4 or 5 hours or more. The heated final implant is cooled. If the article is not in final shape, it can be machined into final implant shape. The implant can be a tibial insert for total knee arthroplasty. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is direct compression molded onto a porous surface into final implant shape. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is layered onto a second layer of vitamin E blended UHMWPE without peroxides and direct compression molded onto a porous surface into final implant shape. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is direct compression molded onto a porous surface into final implant shape. The final implant is heated for a period of time. The heating can be performed at 130° C., 150° C., 160° C., 170° C., 180° C., 190° C., 200° C., 210° C., 220° C. or more. The heating can be performed for 1, 2, 3, 4 or 5 hours or more. The heated final implant is cooled. The final implant is packaged and sterilized by irradiation or gas sterilization.

In some embodiments, ultrahigh molecular weight polyethylene is blended with vitamin E and 2,5-dimethyl-2,5-Di-(t-butylperoxy)hexyne-3. The vitamin E is concentration is 0.5 wt %, 0.6 wt %, 0.8 wt %, 1 wt % or more. The peroxide concentration is 0.5 wt %, 1 wt %, 1.5 wt % or more. The blend is layered onto another layer of vitamin E blended UHMWPE and direct compression molded onto a porous surface into final implant shape. The final implant is heated for a period of time. The heating can be performed at 130° C., 150° C., 160° C., 170° C., 180° C., 190v, 200° C., 210° C., 220° C. or more. The heating can be performed for 1, 2, 3, 4 or 5 hours or more. The heated final implant is cooled. The final implant is packaged and sterilized by irradiation or gas sterilization.

Diffusion of Cross-Linking Agent(s) and Antioxidant(s) into Consolidated Polymeric Material for Cross-Linking

In some embodiments of this invention, cross-linking agent(s) and/or antioxidant(s) can be incorporated into polymeric materials by diffusion after consolidation.

In one embodiment, polymeric material without antioxidants is blended with one or more crosslinking agent(s). The blend is consolidated into an implant preform. At least one cross-linking agent can be a peroxide. The peroxide(s) can be chosen such that the initiation temperatures of the peroxides are substantially less than the molding temperature(s). This is such that the consolidated polymeric blend is substantially cross-linked. Then, one or more antioxidants are diffused into the consolidated blend by immersing the blend in the pure antioxidant(s) or a solution of the antioxidant(s). Alternatively the consolidated polymeric blend is annealed after doping with antioxidants through diffusion to increase the depth of penetration of antioxidants in the consolidated polymeric blend. The implant preform is machined to obtain a final implant. The final implant is packaged and sterilized by irradiation or gas sterilization.

In one embodiment, polymeric material without antioxidants is blended with one or more crosslinking agent(s). The blend is consolidated into an implant preform. At least one cross-linking agent can be a peroxide. The peroxide(s) can be chosen such that the initiation temperatures of the peroxides are substantially higher than the molding temperature(s). This is such that the consolidated polymeric blend is not substantially cross-linked. Then, one or more antioxidants are diffused into the consolidated blend by immersing the blend in the pure antioxidant(s) or a solution of the antioxidant(s). A heating step at a temperature above the initiation temperature(s) of the peroxide(s) can be used to cross-link the polymeric material before, during or after the diffusion of the antioxidant. The implant preform is machined to obtain a final implant. The final implant is packaged and sterilized by irradiation or gas sterilization.

In one embodiment, polymeric material blended with one or more antioxidant(s) is consolidated into an implant preform. Then, one or more antioxidant(s) and/or one or more crosslinking agent(s) are diffused into the consolidated blend by immersing the blend in the pure antioxidant(s) and/or cross-linking agent(s) and/or a solution of the antioxidant(s) and/or cross-linking agent(s). At least one cross-linking agent can be a peroxide. A heating step at a temperature above the initiation temperatures of the peroxides can be used to cross-link the polymeric material before, during or after the diffusion of the antioxidant(s) and/or cross-linking agent(s). The implant preform is machined to obtain a final implant. The final implant is packaged and sterilized by irradiation or gas sterilization.

Diffusion of the antioxidant(s) can be comprised of two or more steps involving immersing the polymeric material in pure antioxidant(s) followed by the homogenization of the antioxidant(s) by an annealing step above or below the melting point of the polymeric material.

Diffusion of the crosslinking agent(s) can be comprised of two or more steps involving immersing the polymeric material in pure crosslinking agent(s) followed by the homogenization of the crosslinking agent(s) by an annealing step above or below the melting point of the polymeric material. The annealing step can be used to decompose the peroxides to cross-link the polymeric material. The annealing step for homogenization may be separate from the annealing step for cross-linking; or these two steps may be combined.

The diffusion of the antioxidant(s) and crosslinking agent(s) can be performed simultaneously or in any order.

In one embodiment, polymeric material blended with one or more antioxidant(s) is consolidated into an implant preform. Then, one or more crosslinking agent(s) are diffused into the consolidated blend by immersing the blend in the pure crosslinking agent(s) or one or more solution(s) of the cross-linking agent(s). At least one cross-linking agent can be a peroxide. A heating step at a temperature above the initiation temperatures of the peroxides can be used to cross-link the polymeric material during or after the diffusion of the cross-linking agent(s). The implant preform can be machined to obtain a final implant. The final implant can be packaged and sterilized by irradiation or gas sterilization.

In one embodiment, polymeric material blended with one or more antioxidant(s) is consolidated into an implant preform. Then, one or more crosslinking agent(s) are diffused into the consolidated blend by immersing the blend in the pure crosslinking agent(s) or one or more solution(s) of the cross-linking agent(s). At least one cross-linking agent can be a peroxide. Some parts of the implant preform can be machined to reduce the amount of cross-linking agent(s) on the surfaces. A heating step at a temperature above the initiation temperatures of the peroxides can be used to cross-link the polymeric material during or after the diffusion of the cross-linking agent(s). The implant preform can be machined to obtain a final implant. The final implant can be packaged and sterilized by irradiation or gas sterilization.

In one embodiment, polymeric material blended with one or more antioxidant(s) is consolidated into an implant preform. Then, one or more crosslinking agent(s) are diffused into the consolidated blend by immersing the blend in the pure crosslinking agent(s) or one or more solution(s) of the cross-linking agent(s). At least one cross-linking agent can be a peroxide. Some surfaces of the implant preform can be contacted with an extraction environment to reduce the amount of crosslinking agent(s) on the surfaces. A heating step at a temperature above the initiation temperatures of the peroxides can be used to cross-link the polymeric material during or after the diffusion of the cross-linking agent(s). The implant preform can be machined to obtain a final implant. The final implant can be packaged and sterilized by irradiation or gas sterilization.

In one aspect, the invention provides methods of making oxidation resistant and cross-linked polymeric material comprising: (a) blending a polymeric material with one or more antioxidant(s); (b) consolidating the blend; and (c) diffusing one or more crosslinking agent(s) into the consolidated antioxidant-blended polymeric material.

In one aspect, the invention provides methods of making oxidation resistant and highly cross-linked polymeric material comprising: (a) blending a polymeric material with one or more antioxidant(s); (b) consolidating the blend; and (c) diffusing one or more crosslinking agent(s) into the consolidated antioxidant-blended polymeric material.

In one aspect, the invention provides methods of making oxidation resistant and highly cross-linked polymeric material comprising: (a) blending a polymeric material with one or more antioxidant(s); (b) consolidating the blend; and (c) diffusing one or more peroxide(s) into the consolidated antioxidant-blended polymeric material at or above the T₁₀ of the peroxide(s).

In a preferred embodiment, ultrahigh molecular weight polyethylene blended with vitamin E is consolidated into an implant preform. Then, dicumyl peroxide is diffused into the consolidated blend by immersing the blend in the pure crosslinking agent(s) or one or more solution(s) of the cross-linking agent(s). Diffusion can be performed at 100° C., 110° C., 120° C., 130° C., 140° C., 150° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the consolidated and cross-linked antioxidant-blended polymeric material is machined into final implant shape. The implant is packaged and sterilized. Sterilization is done by a gas sterilization method or by ionizing radiation.

In a preferred embodiment, ultrahigh molecular weight polyethylene blended with vitamin E is consolidated into an implant preform onto a porous metal surface, thus forming an interlocked hybrid material. Then, dicumyl peroxide is diffused into the interlocked hybrid material by immersing the blend in the pure crosslinking agent(s) or one or more solution(s) of the cross-linking agent(s). Diffusion can be performed at 100° C., 110° C., 120° C., 130° C., 140° C., 150° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the consolidated and cross-linked interlocked hybrid material is machined into final implant shape. The implant is packaged and sterilized. Sterilization is done by a gas sterilization method or by ionizing radiation.

In a most preferred embodiment, ultrahigh molecular weight polyethylene blended with vitamin E is consolidated into an implant preform onto porous metal, thus forming an interlocked hybrid material. Then, dicumyl peroxide is diffused into the interlocked hybrid material by immersing the interlocked hybrid material in the pure crosslinking agent(s) or one or more solution(s) of the cross-linking agent(s). Diffusion can be performed at 40° C., 50° C., 60° C., 70° C., 80° C., 90° C., 100° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the cross-linked interlocked hybrid material is heated for a period of time, then cooled. Heating can be performed at 100° C., 110° C., 120° C., 130° C., 140° C., 150° C. or more. Then, the cross-linked interlocked hybrid material is machined into final implant shape. The implant is packaged and sterilized. Sterilization is done by a gas sterilization method or by ionizing radiation.

In any of the embodiments, only a part of the polymeric material may be contacted with the medium for diffusion. One method of achieving this is to contact the desired part of the polymeric material, for example the surface or parts of the surface with the medium or masking parts of the polymeric material when contacting the diffusion medium.

In a most preferred embodiment, ultrahigh molecular weight polyethylene blended with vitamin E is consolidated into an implant preform. Then, dicumyl peroxide is diffused into the consolidated blend by immersing the blend in the pure crosslinking agent(s) or one or more solution(s) of the cross-linking agent(s). Diffusion can be performed at 40° C., 50° C., 60° C., 70° C., 80° C., 90° C., 100° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the consolidated and cross-linked antioxidant-blended polymeric material is heated for a period of time, then cooled. Heating can be performed at 100° C., 110° C., 120° C., 130° C., 140° C., 150° C. or more. Then, the consolidated and cross-linked antioxidant-blended polymeric material is machined into final implant shape. The implant is packaged and sterilized. Sterilization is done by a gas sterilization method or by ionizing radiation.

In a preferred embodiment, ultrahigh molecular weight polyethylene blended with vitamin E is consolidated into an implant preform. Then, 2,5-dimethyl-2,5-Di-(t-butylperoxy)hexyne-3 is diffused into the consolidated blend by immersing the blend in the pure crosslinking agent(s) or one or more solution(s) of the cross-linking agent(s). Diffusion can be performed at 100° C., 110° C., 120° C., 130° C., 140° C., 150° C., 160° C., 170° C., 180° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the consolidated and cross-linked antioxidant-blended polymeric material is machined into final implant shape. The implant is packaged and sterilized. Sterilization is done by a gas sterilization method or by ionizing radiation.

In a preferred embodiment, ultrahigh molecular weight polyethylene blended with vitamin E is consolidated into an implant preform onto a porous metal surface, thus forming an interlocked hybrid material. Then, 2,5-dimethyl-2,5-Di-(t-butylperoxy)hexyne-3 is diffused into the interlocked hybrid material by immersing the hybrid material in the pure crosslinking agent(s) or one or more solution(s) of the cross-linking agent(s). Diffusion can be performed at 100° C., 110° C., 120° C., 130° C., 140° C., 150° C., 160° C., 170° C., 180° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the cross-linked interlocked hybrid material is machined into final implant shape. The implant is packaged and sterilized. Sterilization is done by a gas sterilization method or by ionizing radiation.

In a most preferred embodiment, ultrahigh molecular weight polyethylene blended with vitamin E is consolidated into an implant preform. Then, 2,5-dimethyl-2,5-Di-(t-butylperoxy)hexyne-3 is diffused into the consolidated blend by immersing the blend in the pure crosslinking agent(s) or one or more solution(s) of the cross-linking agent(s). Diffusion can be performed at 40° C., 50° C., 60° C., 70° C., 80° C., 90° C., 100° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the consolidated and cross-linked antioxidant-blended polymeric material is heated for a period of time, then cooled. Heating can be performed at 130° C., 140° C., 150° C., 160° C., 170° C., 180° C., 190° C. or more. Then, the consolidated and cross-linked antioxidant-blended polymeric material is machined into final implant shape. The implant is packaged and sterilized. Sterilization is done by a gas sterilization method or by ionizing radiation.

In a most preferred embodiment, ultrahigh molecular weight polyethylene blended with vitamin E is consolidated into an implant preform onto a porous surface, thus forming an interlocked hybrid material. Then, 2,5-dimethyl-2,5-Di-(t-butylperoxy)hexyne-3 is diffused into the interlocked hybrid material by immersing the hybrid material in the pure crosslinking agent(s) or one or more solution(s) of the cross-linking agent(s). Diffusion can be performed at 40° C., 50° C., 60° C., 70° C., 80° C., 90° C., 100° C. or more. The vitamin E concentration can be 0.1 wt %, 0.2 wt %, 0.3 wt %, 0.4 wt %, 0.5 wt %, 0.6 wt %, 0.7 wt %, 0.8 wt %, 0.9 wt %, 1.0 wt % or more. The peroxide can be diffused for 10 minutes to 24 hours, more preferably 1 hour to 8 hours, most preferably 4 hours. Then, the cross-linked interlocked hybrid material is heated for a period of time, then cooled. Heating can be performed at 130° C., 140° C., 150° C., 160° C., 170° C., 180° C., 190° C. or more. Then, the cross-linked interlocked hybrid material is machined into final implant shape. The implant is packaged and sterilized. Sterilization is done by a gas sterilization method or by ionizing radiation.

In one aspect, the invention provides methods of making oxidation resistant and highly cross-linked polymeric material comprising: (a) blending a polymeric material with one or more antioxidant(s); (b) consolidating the blend; and (c) diffusing one or more peroxide(s) into the consolidated antioxidant-blended polymeric material at or above the T₁ of the peroxide(s).

In one aspect, the invention provides methods of making oxidation resistant and cross-linked polymeric material comprising: (a) blending a polymeric material with one or more antioxidant(s); (b) consolidating the blend; (c) diffusing one or more crosslinking agent(s) into the consolidated antioxidant-blended polymeric material; and (d) heating the polymeric material.

In one aspect, the invention provides methods of making oxidation resistant and highly cross-linked polymeric material comprising: (a) blending a polymeric material with one or more antioxidant(s); (b) consolidating the blend; (c) diffusing one or more crosslinking agent(s) into the consolidated antioxidant-blended polymeric material; and (d) heating the polymeric material.

In one aspect, the invention provides methods of making oxidation resistant and highly cross-linked polymeric material comprising: (a) blending a polymeric material with one or more antioxidant(s); (b) consolidating the blend; (c) diffusing one or more peroxide(s) into the consolidated antioxidant-blended polymeric material; and (d) heating the polymeric material.

In one aspect, the invention provides methods of making oxidation resistant and cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) heating the medical implant preform; and (e) machining the medical implant preform, thereby forming an oxidation resistant and substantially cross-linked medical implant.

In one aspect, the invention provides methods of making oxidation resistant and highly cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) heating the medical implant preform; and (e) machining the medical implant preform, thereby forming an oxidation resistant and highly cross-linked medical implant.

In one aspect, the invention provides methods of making oxidation resistant and highly cross-linked medical implant comprising: (a) blending a polymeric material with one or more antioxidant(s); (b) consolidating the blend, thereby forming a medical implant preform; (c) diffusing one or more peroxide(s) into the consolidated antioxidant-blended medical implant preform; (d) heating the medical implant preform; and (e) machining the medical implant preform, thereby forming an oxidation resistant and highly cross-linked medical implant.

In one aspect, the invention provides methods of making sterile, oxidation resistant and cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) heating the medical implant preform, thereby forming a substantially cross-linked medical implant preform; (e) machining the medical implant preform, thereby forming an oxidation resistant and substantially cross-linked medical implant; and (f) sterilizing by gas sterilization of radiation sterilization, thereby forming a sterile, oxidation resistant and substantially cross-linked medical implant.

In one aspect, the invention provides methods of making sterile, oxidation resistant and highly cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) heating the medical implant preform, thereby forming a highly cross-linked medical implant preform; (e) machining the medical implant preform, thereby forming an oxidation resistant and highly cross-linked medical implant; and (f) sterilizing by gas sterilization or radiation sterilization, thereby forming a sterile, oxidation resistant and highly cross-linked medical implant.

In one aspect, the invention provides methods of making oxidation resistant and cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) machining the medical implant preform, thereby forming an oxidation resistant medical implant; and (e) heating the medical implant, thereby forming an oxidation resistant and substantially cross-linked medical implant.

In one aspect, the invention provides methods of making oxidation resistant and highly cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) machining the medical implant preform, thereby forming an oxidation resistant medical implant; and (e) heating the medical implant, thereby forming an oxidation resistant and highly cross-linked medical implant.

In one aspect, the invention provides methods of making sterile, oxidation resistant and cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) machining the medical implant preform, thereby forming an oxidation resistant medical implant; (e) heating the medical implant, thereby forming an oxidation resistant and substantially cross-linked medical implant; and (f) sterilizing by gas sterilization or radiation sterilization, thereby forming a sterile, oxidation resistant and substantially cross-linked medical implant.

In one aspect, the invention provides methods of making sterile, oxidation resistant and highly cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) machining the medical implant preform, thereby forming an oxidation resistant medical implant; (e) heating the medical implant, thereby forming an oxidation resistant and highly cross-linked medical implant; and (f) sterilizing by gas sterilization or radiation sterilization, thereby forming a sterile, oxidation resistant and highly cross-linked medical implant.

In one aspect, the invention provides methods of making sterile, oxidation resistant and highly cross-linked medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) machining the medical implant preform, thereby forming an oxidation resistant medical implant; (e) heating the medical implant, thereby forming an oxidation resistant and highly cross-linked medical implant; and (f) sterilizing by gas sterilization or radiation sterilization, thereby forming a sterile, oxidation resistant and highly cross-linked medical implant.

In one aspect, the invention provides methods of making sterile, oxidation resistant and wear resistant medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) heating the medical implant preform, thereby forming an oxidation resistant and wear resistant medical implant preform; (e) machining the medical implant preform, thereby forming an oxidation resistant and wear resistant medical implant; and (f) sterilizing by gas sterilization or radiation sterilization, thereby forming a sterile, oxidation resistant and wear resistant medical implant.

In one aspect, the invention provides methods of making sterile, oxidation resistant and highly wear resistant medical implant comprising: (a) blending a polymeric material with one or more antioxidant(s); (b) consolidating the blend, thereby forming a medical implant preform; (c) diffusing one or more crosslinking agent(s) into the consolidated antioxidant-blended medical implant preform; (d) heating the medical implant preform, thereby forming an oxidation resistant and wear resistant medical implant preform; (e) machining the medical implant preform, thereby forming an oxidation resistant and highly wear resistant medical implant; and (f) sterilizing by gas sterilization or radiation sterilization, thereby forming a sterile, oxidation resistant and highly wear resistant medical implant.

In some embodiments, some of the unreacted cross-linking agent(s) and their byproducts can be extracted from the surface(s) of the polymeric material, implant, or implant preform after diffusion. Extraction from the surface can be done before or after any process step. For example, it can be done after heating to close to or above the decomposition temperature of the peroxide(s) used as cross-linking agent(s). Extraction can be done in solvent(s), emulsion (s), gas(es) (can be inert gas) or supercritical fluid(s) or combinations thereof for sufficient period of time to remove at least 10% of the crosslinking agent(s) and/or its byproducts in the first 1 millimeter or the entire bulk. Extraction can be done at high temperature on under elevated pressure, atmospheric pressure, partial pressure or vacuum to evaporate the byproducts. Extraction can remove 0.1 wt % to 100 wt % of the peroxide and/or its byproducts from the first 1 millimeter of the sample, from the first 2 millimeters of the sample, from the first 3 millimeter of the sample or more or the bulk of the sample. In some embodiments, polymeric material blended with one or more antioxidant(s) is consolidated into an implant preform. Then, one or more crosslinking agent(s) are diffused into the consolidated blend by immersing the blend in the pure crosslinking agent(s) or one or more solution(s) of the cross-linking agent(s). At least one cross-linking agent can be a peroxide. Some of the peroxide(s) and/or its byproducts can be extracted from the surface(s). A heating step at a temperature above the initiation temperatures of the peroxides can be used to cross-link the polymeric material during or after the diffusion of the cross-linking agent(s). The implant preform can be machined to obtain a final implant. The final implant can be packaged and sterilized by irradiation or gas sterilization.

In some embodiments, it is desirable to diffuse cross-linking agent(s) into consolidated polymeric material or antioxidant blends of polymeric material to avoid exposing cross-linking agent(s) to the high temperatures encountered during consolidation. For example, T₁ of benzoyl peroxide is 91° C. and its T₁₀ is 73° C. While consolidation of UHMWPE in the presence of this peroxide at temperatures around 180° C. may cause very fast disassociation/decomposition and oxidation and degradation of the polymer, diffusion of benzoyl peroxide into consolidated UHMWPE at temperatures ranging from room temperature to 100° C., more preferably between room temperature and 70° C. can result in a desired amount of cross-linking agent in the surface of the polymeric material and enable the use of this peroxide to be used in obtaining a mechanically integral, cross-linked UHMWPE. The disassociation/decomposition of the peroxide and cross-linking of the polymeric material can be simultaneous with the diffusion at temperatures where disassociation/decomposition rates are high, for example, 90° C., or can be accomplished after diffusion, where diffusion is achieved at temperatures where disassociation/decomposition rates are low, for example, 60° C., and diffusion is followed by a heating step at one or more temperature(s) where disassociation/decomposition rates are high, for example 120° C.

In other embodiments, the cross-linking of the polymeric material using peroxide(s) is complete by the time the diffusion is complete—that is, as the peroxide diffuses into UHMWPE, it also dissociates into free radicals and causes the cross-linking during the diffusion. In some embodiments, the extent of cross-linking will be sufficient by the time diffusion is complete; in others the process is completed by additional heating to achieve the desired cross-link density. The diffusion and/or homogenization temperatures can be chosen below, at, or above the peroxide initiation temperature such that cross-linking can take place during diffusion and/or homogenization.

In some embodiments, the cross-link density will have a gradient near the surface of the UHMWPE.

In one embodiment, polymeric material is blended with one or more antioxidant(s) and one or more crosslinking agent(s). The blend is consolidated into an implant preform. In this embodiment, at least one cross-linking agent can be a peroxide. The peroxide(s) can be chosen such that the initiation temperatures of the peroxides are substantially less than the molding temperature(s). This is such that the consolidated polymeric blend is substantially cross-linked. Then, one or more antioxidant(s) are diffused into the consolidated blend by immersing the blend in the pure antioxidant(s) or a solution of the antioxidant(s). The implant preform is machined to obtain a final implant. The final implant is packaged and sterilized by irradiation or gas sterilization.

In one embodiment, polymeric material is blended with one or more antioxidant(s). The blend is consolidated into an implant preform. Then, one or more crosslinking agent(s) are diffused into the consolidated blend by immersing the blend in the pure crosslinking agents or a solution of the crosslinking agent(s). At least one cross-linking agent can be a peroxide. The peroxides can be chosen such that the initiation temperatures of the peroxides are substantially less than the diffusion temperature. This is such that the consolidated polymeric blend is substantially cross-linked during the diffusion of the peroxide(s). The implant preform is machined to obtain a final implant. The final implant is packaged and sterilized by irradiation or gas sterilization.

In one embodiment, polymeric material is blended with one or more antioxidant(s). The blend is consolidated into an implant preform or consolidated first and then machined into an implant preform. Then, one or more crosslinking agent(s) are diffused into the implant preform by immersing the implant preform in the pure crosslinking agent(s) or a solution of the crosslinking agent(s). The cross-linking agent can be chosen from peroxides. The peroxide(s) can be chosen such that the initiation temperatures of the peroxides are substantially higher than the diffusion temperature. This is such that the consolidated implant preform is not substantially cross-linked during the diffusion of the peroxide(s). The implant preform can then be substantially cross-linked by heating the peroxide-diffused consolidated polymeric blend to about or above the initiation temperature of the peroxide(s). Heating and/or annealing can be done for 0.1 hours to 1 hour, 2 hours, 3 hours, 4 hours, 24 hours, or more. The implant preform is machined to obtain a final implant. The final implant is packaged and sterilized by irradiation or gas sterilization.

In one embodiment, polymeric material is blended with one or more antioxidants. The blend is consolidated into an implant preform or consolidated and then machined into an implant preform. Then, one or more crosslinking agent(s) and one or more antioxidants are diffused into the consolidated blend by immersing the blend in the pure crosslinking agents or a solution of the crosslinking agent(s). The cross-linking agent can be chosen from peroxides. The peroxide(s) can be chosen such that the initiation temperatures of the peroxides are substantially higher than the diffusion temperature. This is such that the consolidated polymeric blend is not substantially cross-linked during the diffusion of the peroxide(s). The implant perform can then be substantially cross-linked by heating the peroxide-diffused consolidated polymeric blend to about or above the initiation temperature of the peroxide(s). The implant preform is machined to obtain a final implant. The final implant is packaged and sterilized by irradiation or gas sterilization.

In any of the embodiments, the diffusion of different components can be performed simultaneously or in subsequent steps in any order. All cross-linking can be done either in air, or any vacuum, or in inert gas, or sensitizing gas, or a mixture thereof.

In any of the embodiments, the diffusion of the antioxidant(s) and/or cross-linking agent(s) can be performed in pure form or in solution or emulsion of the compounds. Emulsion of the antioxidant(s) and/or cross-linking agent(s) can be done with the aid of emulsifying agent(s), for example Tween 20 or Tween 80. Similarly, antioxidant(s) and/or cross-linking agent(s) can be dissolved in a solvent or a mixture of solvents for diffusion.

In one aspect, the invention provides methods of making an oxidation resistant, cross-linked polymeric material comprising: (a) blending a polymeric material with one or more peroxide(s), thereby forming an peroxide blended polymeric material; (b) consolidating the polymeric material, thereby forming a peroxide blended, consolidated polymeric material; (c) machining the peroxide blended, consolidated polymeric material; (c) diffusing one or more antioxidant(s) into the peroxide blended consolidated polymeric material; (d) heating the antioxidant diffused, peroxide blended consolidated polymeric material, thereby forming an oxidation resistant, cross-linked polymeric material. This implant is then packaged and sterilized.

In one aspect, the invention provides methods of making an oxidation resistant, cross-linked polymeric material comprising: (a) blending a polymeric material with one or more peroxide(s) and one or more antioxidant(s), thereby forming an peroxide and antioxidant blended polymeric material; (b) consolidating the polymeric material, thereby forming a peroxide blended, consolidated, crosslinked polymeric material; (c) machining the peroxide blended, consolidated, crosslinked polymeric material; (d) diffusing one or more antioxidant(s) into the peroxide blended consolidated polymeric material; (e) heating the antioxidant diffused, peroxide blended consolidated polymeric material, thereby forming an oxidation resistant, cross-linked polymeric material. This implant is then packaged and sterilized.

### Radiation Cross-Linking

Exposure to irradiation is known to cross-link most polymeric materials. Radiation cross-linking of UHMWPE is used in reducing the wear rate of UHMWPE used in joint replacements.

In some embodiments, the cross-linking agent and antioxidant-doped UHMWPE can be further irradiated to further cross-link the polymeric material and/or sterilize the implant. In some embodiments the peroxide(s) and/or vitamin E containing UHMWPE is irradiated to further cross-link the material and/or sterilize the implant.

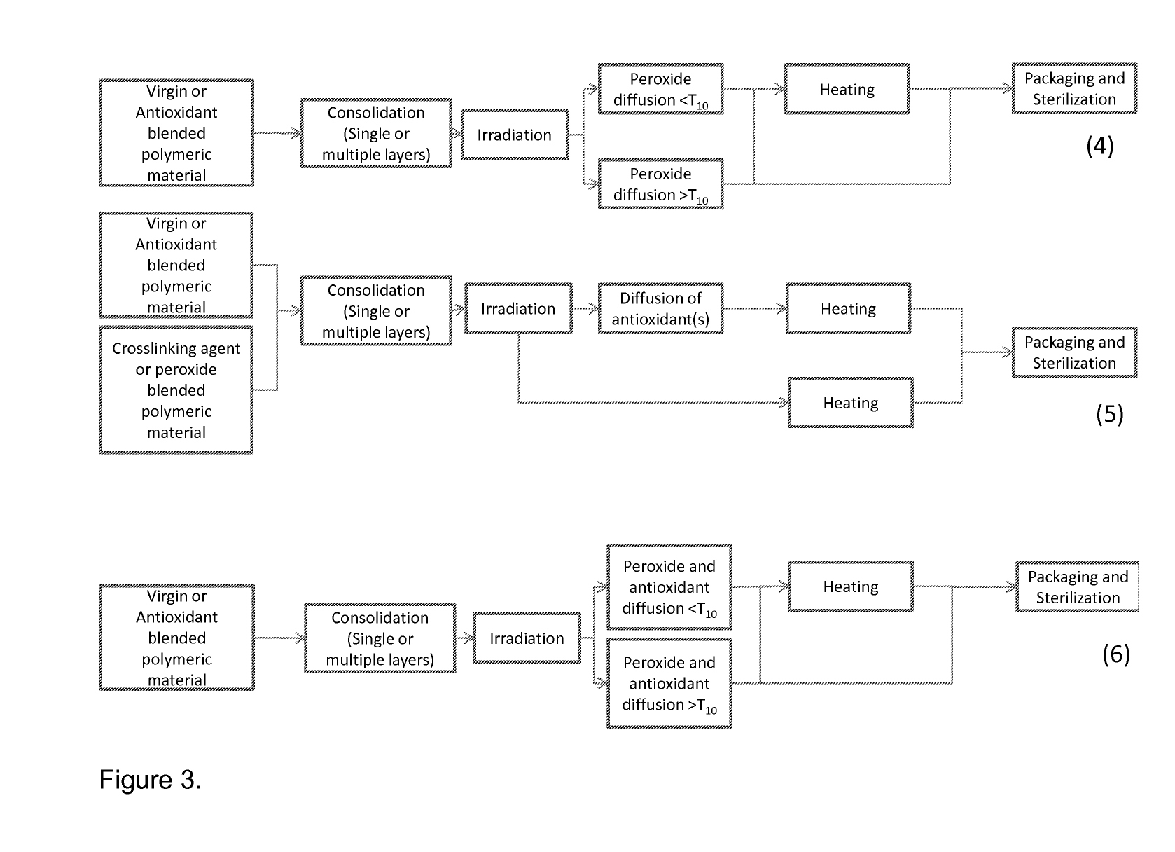

Some schemes for cross-linking polymeric material by a combination of irradiation and crosslinking agents are shown in FIG. 3.

In some embodiments, an antioxidant containing UHMWPE can be irradiated to cross-link the polymeric material; then the cross-linked polymeric material is further cross-linked by incorporating and activating cross-linking agents, for example, peroxide(s).

In some embodiments, irradiation of an antioxidant-containing polymeric material is performed to cause grafting of some or all of the antioxidant or antioxidant(s) onto the polymeric material.

In one embodiment, the polymeric material is blended with one or more antioxidant(s). The polymeric blend is consolidated into an implant preform. The implant preform is irradiated. Then, one or more crosslinking agent(s) are diffused into the consolidated blend by immersing the blend in the pure crosslinking agents or a solution of the crosslinking agent(s). The cross-linking agent can be chosen from peroxides. The peroxides can be chosen such that the initiation temperatures of the peroxides are substantially higher than the diffusion temperature. This is such that the consolidated polymeric blend is not substantially cross-linked during the diffusion of the peroxide(s). The implant perform can then be substantially cross-linked by heating the peroxide-diffused consolidated polymeric blend to about or above the initiation temperature of the peroxide(s). The implant preform is machined to obtain a final implant before or after irradiation, before and after diffusion of the cross-linking agent or before or after the heating for cross-linking. The final implant is packaged and sterilized by irradiation or gas sterilization.

In one aspect, the invention provides methods of making an oxidation resistant, substantially cross-linked polymeric material comprising: (a) blending a polymeric material with one or more antioxidant(s), thereby forming an oxidation resistant polymeric material; (b) consolidating the polymeric material, thereby forming an oxidation resistant, consolidated polymeric material; (c) irradiating the consolidated polymeric material; (d); diffusing one or more peroxide(s) into the oxidation resistant, irradiated consolidated polymeric material; (e) heating the oxidation resistant, consolidated polymeric material, thereby forming an oxidation resistant, substantially cross-linked, consolidated polymeric material.

In one aspect, the invention provides methods of making an oxidation resistant, highly cross-linked polymeric material comprising: (a) blending a polymeric material with one or more antioxidant(s), thereby forming an oxidation resistant polymeric material; (b) consolidating the polymeric material, thereby forming an oxidation resistant, consolidated polymeric material; (c) irradiating the consolidated polymeric material; (d) diffusing one or more peroxide(s) into the oxidation resistant, irradiated consolidated polymeric material; and (e) heating the oxidation resistant, consolidated polymeric material, thereby forming an oxidation resistant, highly cross-linked, consolidated polymeric material.

In one aspect, the invention provides methods of making an oxidation resistant, substantially cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) irradiating the consolidated polymeric material; (d) diffusing one or more peroxide(s) into the oxidation resistant, irradiated consolidated polymeric material; (e) heating the oxidation resistant, consolidated polymeric material, thereby forming an oxidation resistant; and (f) machining, thereby forming an oxidation resistant, substantially cross-linked medical implant.

In one aspect, the invention provides methods of making an oxidation resistant, highly cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) irradiating the consolidated polymeric material; (d) diffusing one or more peroxide(s) into the oxidation resistant, irradiated consolidated polymeric material; (e) heating the oxidation resistant, consolidated polymeric material, thereby forming an oxidation resistant; and (f) machining, thereby forming an oxidation resistant, highly cross-linked medical implant.

In one aspect, the invention provides method of making a sterile, oxidation resistant, substantially cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) irradiating the consolidated polymeric material; (d) diffusing one or more peroxide(s) into the oxidation resistant, consolidated, irradiated polymeric material; (e) heating; (f) machining, thereby forming an oxidation resistant, substantially cross-linked medical implant; and (g) sterilizing the oxidation resistant, substantially cross-linked medical implant, thereby forming a sterile, oxidation resistant, substantially cross-linked medical implant.

In one aspect, the invention provides method of making a sterile, oxidation resistant, highly cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) irradiating the consolidated polymeric material; (d) diffusing one or more peroxide(s) into the oxidation resistant, consolidated, irradiated polymeric material; (e) heating; (f) machining, thereby forming an oxidation resistant, highly cross-linked medical implant; and (g) sterilizing the oxidation resistant, highly cross-linked medical implant, thereby forming a sterile, oxidation resistant, highly cross-linked medical implant.

In all of the above embodiments where the polymeric material is subjected to ionizing radiation, the step of ionizing radiation can take place after chemical cross-linking using a cross-linking agent such as a peroxide. For instance, in one embodiment polymeric material containing an antioxidant that is also a chemically cross-linked using a peroxide is subjected to irradiation at a temperature between room temperature and the melting point of the polymeric material.

In some embodiments, the cross-linking agent and antioxidant-doped UHMWPE can be further irradiated. Further irradiation may not cause an increase in cross-linking but may cause an increase in wear resistance. In some embodiments the peroxide(s) and/or vitamin E containing UHMWPE is irradiated to increase the wear resistance of the material and/or sterilize the implant.

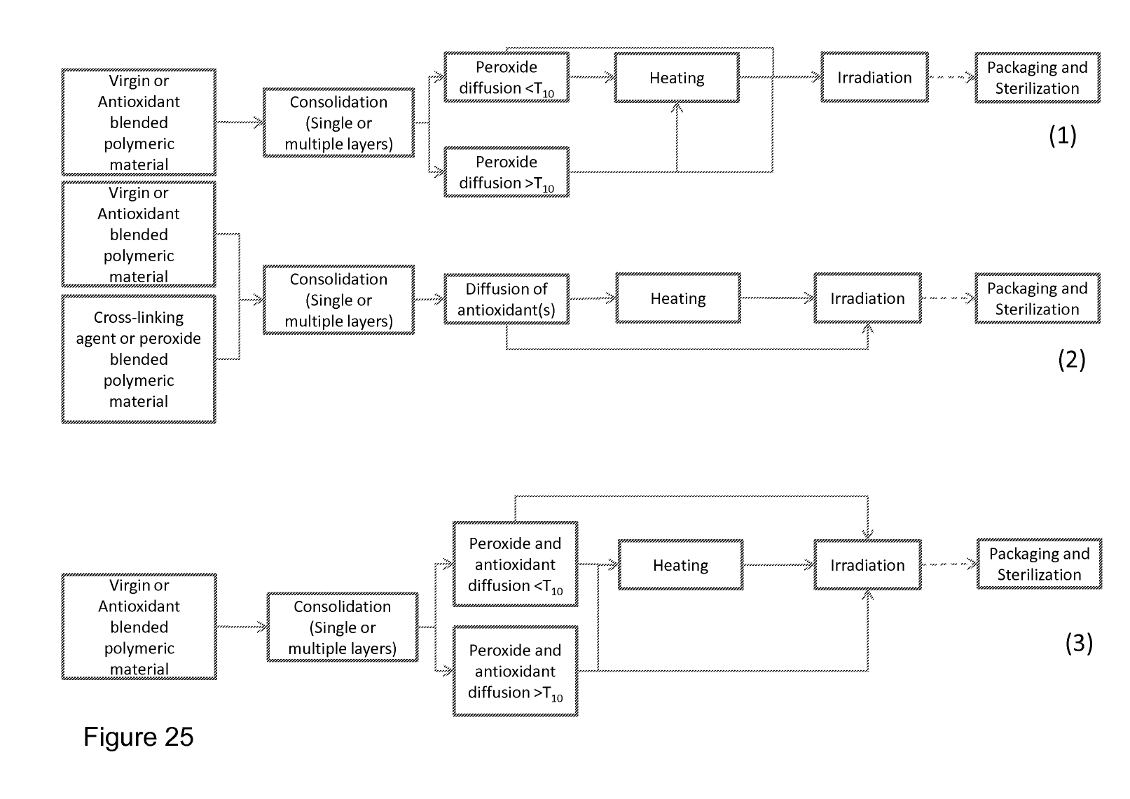

Some schemes for cross-linking polymeric material by a combination of cross-linking agents and irradiation are shown in FIG. 25.

In some embodiments, an antioxidant containing UHMWPE can be cross-linked by incorporating and activating cross-linking agents, for example, peroxide(s). Then, the antioxidant and cross-linking agent containing UHMWPE can be further treated by radiation. If the radiation is used a terminal step, it may also be used for the purpose of sterilization. In some embodiments, the antioxidant and cross-linking agent containing, irradiated UHMWPE can be sterilized by sterilization methods other than radiation, for example gas sterilization.

In some embodiments, irradiation of an antioxidant-containing polymeric material is performed to cause grafting of some or all of the antioxidant or antioxidant(s) onto the polymeric material. In some embodiments, irradiation of a crosslinking agent containing polymeric material can be used to degrade or decompose the cross-linking agent.

In one embodiment, the polymeric material is blended with one or more antioxidant(s). The polymeric blend is consolidated into an implant preform. Then, one or more crosslinking agent(s) are diffused into the consolidated blend by immersing the blend in the pure crosslinking agents or a solution of the crosslinking agent(s). The cross-linking agent can be chosen from peroxides. The peroxides can be chosen such that the initiation temperatures of the peroxides are substantially higher than the diffusion temperature. This is such that the consolidated polymeric blend is not substantially cross-linked during the diffusion of the peroxide(s). The implant perform can then be substantially cross-linked by heating the peroxide-diffused consolidated polymeric blend to about or above the initiation temperature of the peroxide(s). Then the implant preform can be irradiated. The implant preform is machined to obtain a final implant before or after irradiation, before and after diffusion of the cross-linking agent or before or after the heating for cross-linking. The final implant is packaged and sterilized by irradiation or gas sterilization.

In one aspect, the invention provides methods of making an oxidation resistant, cross-linked polymeric material comprising: (a) blending a polymeric material with one or more antioxidant(s), thereby forming an oxidation resistant polymeric material; (b) consolidating the polymeric material, thereby forming an oxidation resistant, consolidated polymeric material; (c); diffusing one or more peroxide(s) into the oxidation resistant, consolidated polymeric material; (d) heating the oxidation resistant, consolidated polymeric material; and (e) irradiating the oxidation resistant, cross-linked consolidated polymeric material, thereby forming an oxidation resistant, cross-linked, consolidated polymeric material.

In one aspect, the invention provides methods of making an oxidation resistant, cross-linked polymeric material comprising: (a) blending a polymeric material with one or more antioxidant(s), thereby forming an oxidation resistant polymeric material; (b) consolidating the polymeric material, thereby forming an oxidation resistant, consolidated polymeric material; (c); diffusing one or more peroxide(s) into the oxidation resistant, consolidated polymeric material; (d) irradiating the consolidated polymeric material; and (e) heating the oxidation resistant, consolidated polymeric material, thereby forming an oxidation resistant, cross-linked, consolidated polymeric material.

In one aspect, the invention provides methods of making an oxidation resistant, cross-linked polymeric material comprising: (a) blending a polymeric material with one or more antioxidant(s), thereby forming an oxidation resistant polymeric material; (b) consolidating the polymeric material, thereby forming an oxidation resistant, consolidated polymeric material; (c) diffusing one or more peroxide(s) into the oxidation resistant, consolidated polymeric material; and (d) irradiating the consolidated polymeric material, thereby forming an oxidation resistant, cross-linked, consolidated polymeric material.

In some embodiments, irradiation can be performed at an elevated temperature and/or the temperature during irradiation can be controlled by the pre-heat temperature and dose rate of irradiation to cause decomposition of the cross-linking agent and cross-linking.

In one aspect, the invention provides methods of making an oxidation resistant, cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) diffusing one or more peroxide(s) into the oxidation resistant, consolidated polymeric material; (d) heating the oxidation resistant, consolidated, peroxide-diffused polymeric material; (e) irradiating the oxidation resistant, consolidated, peroxide-diffused and heated polymeric material; and (f) machining, thereby forming an oxidation resistant, cross-linked medical implant. This implant is then packaged and sterilized.

In one aspect, the invention provides methods of making an oxidation resistant, cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) diffusing one or more peroxide(s) into the oxidation resistant, consolidated polymeric material; (d) irradiating the consolidated, peroxide diffused polymeric material; and (e) heating the oxidation resistant, consolidated, peroxide diffused, irradiated polymeric material; and (f) machining, thereby forming an oxidation resistant, cross-linked medical implant. This implant is then packaged and sterilized.

In one aspect, the invention provides methods of making an oxidation resistant, cross-linked medical implant comprising: (a) blending a polymeric material with one or more antioxidant(s), thereby forming an oxidation resistant polymeric material; (b) consolidating the polymeric material, thereby forming an oxidation resistant, consolidated polymeric material; (c) diffusing one or more peroxide(s) into the oxidation resistant, consolidated polymeric material; (d) irradiating the consolidated, peroxide diffused polymeric material; (e) machining, thereby forming an oxidation resistant, cross-linked medical implant. This implant is then packaged and sterilized.

In some aspects, antioxidant(s) and peroxide(s) or other additives can be diffused into the consolidated polymeric material at the same time or one after the other.

In any of the embodiments, radiation treatment may decrease, not change, or increase the cross-link density.

In one embodiment, the polymeric material is blended with one or more antioxidant(s). The polymeric blend is consolidated into an implant preform. The implant preform is irradiated. Then, one or more crosslinking agent(s) are diffused into the consolidated blend by immersing the blend in the pure crosslinking agents or a solution of the crosslinking agent(s). The cross-linking agent can be chosen from peroxides. The peroxides can be chosen such that the initiation temperatures of the peroxides are substantially lower than the diffusion temperature. This is such that the consolidated polymeric blend is further cross-linked during the diffusion of the peroxide(s). The implant perform can then be substantially cross-linked by heating the peroxide-diffused consolidated polymeric blend to about or above the initiation temperature of the peroxide(s). The implant preform is machined to obtain a final implant before or after irradiation, before and after diffusion of the cross-linking agent or before or after the heating for cross-linking. The final implant is packaged and sterilized by irradiation or gas sterilization.

Irradiation can be done by ionizing irradiation, specifically by electron beam or gamma irradiation. Irradiation temperature can be below, at or above the melting temperatures of the polymeric material or blends of the polymeric material with the antioxidant(s) and/or peroxide(s).

Gamma irradiation or electron radiation may be used. In general, gamma irradiation results in a higher radiation penetration depth than electron irradiation. Gamma irradiation, however, generally provides low radiation dose rate and requires a longer duration of time, which can result in more in-depth and extensive oxidation, particularly if the gamma irradiation is carried out in air. Oxidation can be reduced or prevented by carrying out the gamma irradiation in an inert gas, such as nitrogen, argon, or helium, or under vacuum. Electron irradiation, in general, results in more limited dose penetration depth, but requires less time and, therefore, reduces the risk of extensive oxidation if the irradiation is carried out in air. In addition, if the desired dose levels are high, for instance 20 MRad, the irradiation with gamma may take place over one day, leading to impractical production times. On the other hand, the dose rate of the electron beam can be adjusted by varying the irradiation parameters, such as conveyor speed, scan width, and/or beam power. With the appropriate parameters, a 20 MRad melt-irradiation can be completed in for instance less than 10 minutes. The penetration of the electron beam depends on the beam energy measured by million electron-volts (MeV). Most polymers exhibit a density of about 1 g/cm³, which leads to the penetration of about 1 centimeter with a beam energy of 2-3 MeV and about 4 centimeters with a beam energy of 10 MeV. If electron irradiation is preferred, the desired depth of penetration can be adjusted based on the beam energy. Accordingly, gamma irradiation or electron irradiation may be used based upon the depth of penetration preferred, time limitations and tolerable oxidation levels. Double-sided irradiation using electron beam can increase the overall thickness of the irradiated polymeric material.

In some embodiments low energy electron beam is used to limit the effect of irradiation to a thin surface layer of the polymeric material. The polymeric material may be in any form. For instance it could be in the form of an implant preform or an implant.

Various irradiation methods are defined and described in greater detail below:

### (i) Irradiation in the Molten State (IMS):

Melt-irradiation (MIR), or irradiation in the molten state (“IMS”), is described in detail in U.S. Pat. No. 5,879,400. In the IMS process, the polymer to be irradiated is heated to at or above its melting point. Then, the polymer is irradiated. Following irradiation, the polymer is cooled.

Prior to irradiation, the polymer is heated to at or above its melting temperature and maintained at this temperature for a time sufficient to allow the polymer chains to achieve an entangled state. A sufficient time period may range, for example, from about 5 minutes to about 3 hours. For UHMWPE, the polymer may be heated to a temperature between about 145° C. and about 230° C., preferably about 150° C. to about 200° C.

The temperature of melt-irradiation for a given polymer depends on the differential scanning calorimetry (DSC) (measured at a heating rate of 10° C./min during the first heating cycle) peak melting temperature (“PMT”) for that polymer. In general, the irradiation temperature in the IMS process is at least about 2° C. higher than the PMT, more preferably between about 2° C. and about 20° C. higher than the PMT, and most preferably between about 5° C. and about 10° C. higher than the PMT.

The total dose of irradiation also may be selected as a parameter in controlling the properties of the irradiated polymer. In particular, the dose of irradiation can be varied to control the degree of cross-linking and crystallinity in the irradiated polymer. The total dose may range from about 0.1 MRad to as high as the irradiation level where the changes in the polymer characteristics induced by the irradiation reach a saturation point. For instance, the high end of the dose range could be 20 MRad for the melt-irradiation of UHMWPE, above which dose level the cross-link density and crystallinity are not appreciably affected with any additional dose. The preferred dose level depends on the desired properties that will be achieved following irradiation. Additionally, the level of crystallinity in polyethylene is a strong function of radiation dose level. See Dijkstra et al., Polymer 30: 866-73 (1989). For instance with IMS irradiation, a dose level of about 20 Mrad would decrease the crystallinity level of UHMWPE from about 55% to about 30%. This decrease in crystallinity may be desirable in that it also leads to a decrease in the elastic modulus of the polymer and consequently a decrease in the contact stress when a medical prosthesis made out of the IMS-treated UHMWPE gets in contact with another surface during in vivo use. Lower contact stresses are preferred to avoid failure of the polymer through, for instance, subsurface cracking, delamination, fatigue, etc. The increase in the cross-link density is also desirable in that it leads to an increase in the wear resistance of the polymer, which in turn reduces the wear of the medical prostheses made out of the cross-linked polymer and substantially reduces the amount of wear debris formed in vivo during articulation against a counterface.

In electron beam IMS, the energy deposited by the electrons is converted to heat. This primarily depends on how well the sample is thermally insulated during the irradiation. With good thermal insulation, most of the heat generated is not lost to the surroundings and leads to the adiabatic heating of the polymer to a higher temperature than the irradiation temperature. The heating could also be induced by using a high enough dose rate to minimize the heat loss to the surroundings. In some circumstances, heating may be detrimental to the sample that is being irradiated. Gaseous by-products, such as hydrogen gas when polyethylene is irradiated, are formed during the irradiation. During irradiation, if the heating is rapid and high enough to cause rapid expansion of the gaseous by-products, and thereby not allowing them to diffuse out of the polymer, the polymer may cavitate. The cavitation is not desirable in that it leads to the formation of defects (such as air pockets, cracks) in the structure that could in turn adversely affect the mechanical properties of the polymer and in vivo performance of the device made thereof.

The temperature rise depends on the dose level, level of insulation, and/or dose rate. The dose level used in the irradiation stage is determined based on the desired properties. In general, the thermal insulation is used to avoid cooling of the polymer and maintaining the temperature of the polymer at the desired irradiation temperature. Therefore, the temperature rise can be controlled by determining an upper dose rate for the irradiation. For instance, for the IMS of UHMWPE the dose rate should be less than about 5 Mrad/pass. These considerations for optimization for a given polymer of a given size are readily determined by the person of skill in view of the teachings contained herein.

In embodiments of the present invention in which electron radiation is utilized, the energy of the electrons can be varied to alter the depth of penetration of the electrons, thereby controlling the degree of cross-linking and crystallinity following irradiation. The range of suitable electron energies is disclosed in greater detail in PCT Patent Application Publication No. WO 97/29793. In one embodiment, the energy is about 0.5 MeV to about 12 MeV. In another embodiment the energy is about 1 MeV to 10 MeV. In another embodiment, the energy is 1.7 MeV. Or it can be from 0.5 to 10 MeV in 0.5 MeV intervals. In another embodiment, the energy is about 10 MeV.

### (ii) Cold Irradiation (CIR):

An example of cold irradiation is described in PCT Patent Application Publication No. WO 97/29793, the contents of which is herein incorporated by reference in its entirety. In the cold irradiation process, a polymer is provided at room temperature or below room temperature. Preferably, the temperature of the polymer is about 20° C. Then, the polymer is irradiated. In one embodiment of cold irradiation, the polymer may be irradiated at a high enough total dose and/or at a fast enough dose rate to generate enough heat in the polymer to result in at least a partial melting of the crystals of the polymer.

Gamma irradiation or electron radiation may be used. In general, gamma irradiation results in a higher dose penetration depth than electron irradiation. Gamma irradiation, however, generally requires a longer duration of time, which can result in more in-depth oxidation, particularly if the gamma irradiation is carried out in air. Oxidation can be reduced or prevented by carrying out the gamma irradiation in an inert gas, such as nitrogen, argon, or helium, or under vacuum. Electron irradiation, in general, results in more limited dose penetration depths, but requires less time and, therefore, reduces the risk of extensive oxidation. Accordingly, gamma irradiation or electron irradiation may be used based upon the depth of penetration preferred, time limitations and tolerable oxidation levels.

The total dose of irradiation may be selected as a parameter in controlling the properties of the irradiated polymer. In particular, the dose of irradiation can be varied to control the degree of cross-linking and crystallinity in the irradiated polymer. The preferred dose level depends on the molecular weight of the polymer and the desired properties that will be achieved following irradiation. For instance, to achieve maximum improvement in wear resistance using UHMWPE and the WIAM (warm irradiation and adiabatic melting) or CISM (cold irradiation and subsequent melting) processes, a radiation dose of about 10 Mrad is suggested. To achieve maximum improvement in wear resistance using LDPE and LLDPE, a dose level greater than about 10 Mrad is suggested. In general, increasing the dose level with CIR would lead to an increase in wear resistance. If the CIR is carried out without further post-irradiation thermal treatment such as melting, the crystallinity and elastic modulus of the polymer would increase. Following melting, however, these would decrease to values lower than those prior to irradiation.

Exemplary ranges of acceptable total dosages are disclosed in greater detail in PCT Patent Application Publication No. WO 97/29793, the contents of which is herein incorporated by reference in its entirety. In the embodiments below, UHMWPE is used as the starting polymer. In one embodiment, the total dose is about 0.05 MRad to about 1,000 MRad. In another embodiment, the total dose is about 1 MRad to about 100 MRad. In yet another embodiment, the total dose is about 4 MRad to about 30 MRad. In still other embodiments, the total dose is about 20 MRad or about 15 MRad. If radiation is used as a means of sterilization, generally a dose of up to 40 kGy (4 MRad) is used. But, doses of about 0.1 kGy to 1000 kGy can be used for sterilization, preferably about 10 kGy or 50 kGy, most preferably about 25-40 kGy.

If electron radiation is utilized, the energy of the electrons also is a parameter that can be varied to tailor the properties of the irradiated polymer. In particular, differing electron energies will result in different depths of penetration of the electrons into the polymer. The practical electron energies range from about 0.1 MeV to 16 MeV giving approximate iso-dose penetration levels of 0.5 mm to 8 cm, respectively. A preferred electron energy for maximum penetration is about 10 MeV, which is commercially available through vendors such as Studer (Daniken, Switzerland) or E-Beam Services (New Jersey, USA). The lower electron energies may be preferred for embodiments where a surface layer of the polymer is preferentially cross-linked with gradient in cross-link density as a function of distance away from the surface. A preferred electron energy for surface penetration of electrons is 1.7 MeV.

(iii) Warm Irradiation (WIR):

Warm irradiation is described in detail in PCT Patent Application Publication No. WO 97/29793, the contents of which is herein incorporated by reference in its entirety. In the warm irradiation process, a polymer is provided at a temperature above room temperature and below the melting temperature of the polymer. Then, the polymer is irradiated. In one embodiment of warm irradiation, it has been termed “warm irradiation adiabatic melting” or “WIAM.” In a theoretical sense, adiabatic heating means an absence of heat transfer to the surroundings. In a practical sense, such heating can be achieved by the combination of insulation, irradiation dose rates and irradiation time periods, as disclosed herein and in the documents cited herein. However, there are situations where irradiation causes heating, but there is still a loss of energy to the surroundings. Also, not all warm irradiation refers to an adiabatic heating. Warm irradiation also can have non-adiabatic or partially (such as about 10-75% of the heat generated is lost to the surroundings) adiabatic heating. In all embodiments of WIR, the polymer may be irradiated at a high enough total dose and/or a high enough dose rate to generate enough heat in the polymer to result in at least a partial melting of the crystals of the polymer.

The polymer may be provided at any temperature below its melting point but preferably above room temperature. The temperature selection depends on the specific heat and the enthalpy of melting of the polymer and the total dose level that will be used. The equation is provided in PCT Patent Application Publication No. WO 97/29793 may be used to help calculate the preferred temperature range with the criterion that the final temperature of polymer may be below or above the melting point. Preheating of the polymer to the desired temperature may be done in an inert or non-inert environment.

Exemplary ranges of acceptable total dosages are disclosed in greater detail in WO 97/29793. In one embodiment, the UHMWPE is preheated to about room temperature (about 25° C.) to about 135° C. In one embodiment of WIAM, the UHMWPE is preheated to about 100° C. to just below the melting temperature of the polymer. In another embodiment of WIAM, the UHMWPE is preheated to a temperature of about 100° C. to about 135° C. In yet other embodiments of WIAM, the polymer is preheated to about 120° C. or about 130° C.

In general terms, the pre-irradiation heating temperature of the polymer can be adjusted based on the peak melting temperature (PMT) measure on the DSC at a heating rate of 10° C./min during the first heat. In one embodiment the polymer is heated to about 20° C. to about PMT. In another embodiment, the polymer is preheated to about 90° C. In another embodiment, the polymer is heated to about 100° C. In another embodiment, the polymer is preheated to about 30° C. below PMT and 2° C. below PMT. In another embodiment, the polymer is preheated to about 12° C. below PMT.

In the WIAM embodiment of WIR, the temperature of the polymer following irradiation is at or above the melting temperature of the polymer. Exemplary ranges of acceptable temperatures following irradiation are disclosed in greater detail in WO 97/29793. In one embodiment, the temperature following irradiation is about room temperature to PMT, or about 40° C. to PMT, or about 100° C. to PMT, or about 110° C. to PMT, or about 120° C. to PMT, or about PMT to about 200° C. In another embodiment, the temperature following irradiation is about 145° C. to about 190° C. In yet another embodiment, the temperature following irradiation is about 145° C. to about 190° C. In still another embodiment, the temperature following irradiation is about 150° C.

In WIR, gamma irradiation or electron radiation may be used. In general, gamma irradiation results in a higher dose penetration depth than electron irradiation. Gamma irradiation, however, generally requires a longer duration of time, which can result in more in-depth oxidation, particularly if the gamma irradiation is carried out in air. Oxidation can be reduced or prevented by carrying out the gamma irradiation in an inert gas, such as nitrogen, argon, or helium, or under vacuum. Electron irradiation, in general, results in more limited dose penetration depths, but requires less time and, therefore, reduces the risk of extensive oxidation. Accordingly, gamma irradiation or electron irradiation may be used based upon the depth of penetration preferred, time limitations and tolerable oxidation levels. In the WIAM embodiment of WIR, electron radiation is used.

The total dose of irradiation may also be selected as a parameter in controlling the properties of the irradiated polymer. In particular, the dose of irradiation can be varied to control the degree of cross-linking and crystallinity in the irradiated polymer. Exemplary ranges of acceptable total dosages are disclosed in greater detail in WO 97/29793.

The dose rate of irradiation also may be varied to achieve a desired result. The dose rate is a prominent variable in the WIAM process. In the case of WIAM irradiation of UHMWPE, higher dose rates would provide the least amount of reduction in toughness and elongation at break. The preferred dose rate of irradiation would be to administer the total desired dose level in one pass under the electron-beam. One also can deliver the total dose level with multiple passes under the beam, delivering a (equal or unequal) portion of the total dose at each time. This would lead to a lower effective dose rate.

Ranges of acceptable dose rates are exemplified in greater detail in WO 97/29793. In general, the dose rates will vary between 0.5 Mrad/pass and 50 Mrad/pass. The upper limit of the dose rate depends on the resistance of the polymer to cavitation/cracking induced by the irradiation.

In some embodiments, irradiation of a crosslinking agent-doped polymeric material is performed to initiate free radicals. In some embodiments, heating of the polymer can be performed during irradiation. In some embodiments, heating of the polymer during irradiation can be to a temperature above the initiation temperature of at least one of the peroxides used as cross-linking agent(s). In some embodiments, cross-linking of the polymer can be induced by irradiation and/or heating. In some embodiments, radiation used for sterilization can induce cross-linking of the polymeric material or antioxidant and/or peroxide containing polymeric material. In some embodiments, irradiation of the polymeric material in the presence of the cross-linking agent(s) can cause grafting of the cross-linking agent(s) onto the polymeric material.

In one embodiment, the polymeric material is blended with one or more antioxidant(s). The polymeric blend is consolidated into an implant preform. Then, one or more crosslinking agent(s) are diffused into the consolidated blend by immersing the blend in the pure crosslinking agent(s) or a solution of the crosslinking agent(s). Then the cross-linking reactions are initiated. The cross-linking agent can be chosen from peroxides. The implant preform is irradiated. The implant preform is machined to obtain a final implant before and after diffusion of the cross-linking agent or before or after irradiation. The final implant is packaged and sterilized by irradiation or gas sterilization.

In one embodiment, the polymeric material is blended with one or more antioxidant(s) and one or more crosslinking agent(s). At least one crosslinking agent can be a peroxide. The polymeric blend is consolidated into an implant preform. The implant preform is irradiated. The implant preform is machined to obtain a final implant before and after diffusion of the cross-linking agent or before or after irradiation. Cross-linking can be initiated before or after consolidation. The final implant is packaged and sterilized by irradiation or gas sterilization.

In one embodiment, the polymeric material is blended with one or more antioxidant(s) and one or more crosslinking agent(s). At least one crosslinking agent can be a peroxide. The polymeric blend is consolidated into an implant preform. The implant preform is irradiated at an elevated temperature above or below the melting temperature of the polymeric material. The implant preform is machined to obtain a final implant before and after diffusion of the cross-linking agent or before or after irradiation. The final implant is packaged and sterilized by irradiation or gas sterilization.

In one embodiment, consolidated UHMWPE is irradiated and then doped with a peroxide or a peroxide solution followed by the optional step of thermal treatment to initiate cross-linking.

In some embodiments, machining of a polymeric material can be done at any step after consolidation into a solid article. Multiple machining steps can be used after different steps after consolidation of the polymeric material into a solid article, for example a medical implant preform or a medical implant.

All consolidated material is machined into finished implant, packaged, and sterilized using ionizing radiation and/or with gas sterilization.

In some embodiments, the consolidation uses direct compression molding to achieve a finished implant.

### Silanes as Cross-Linking Agents

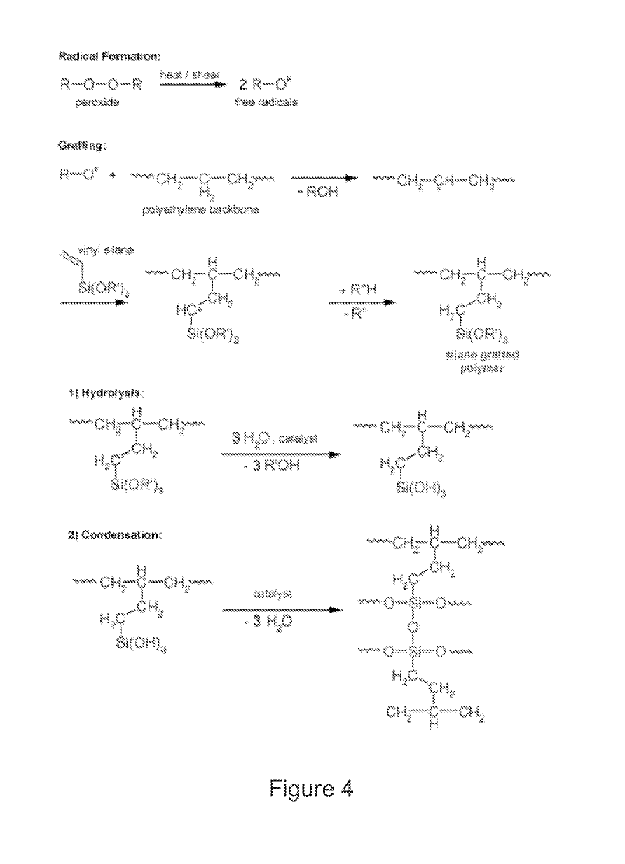

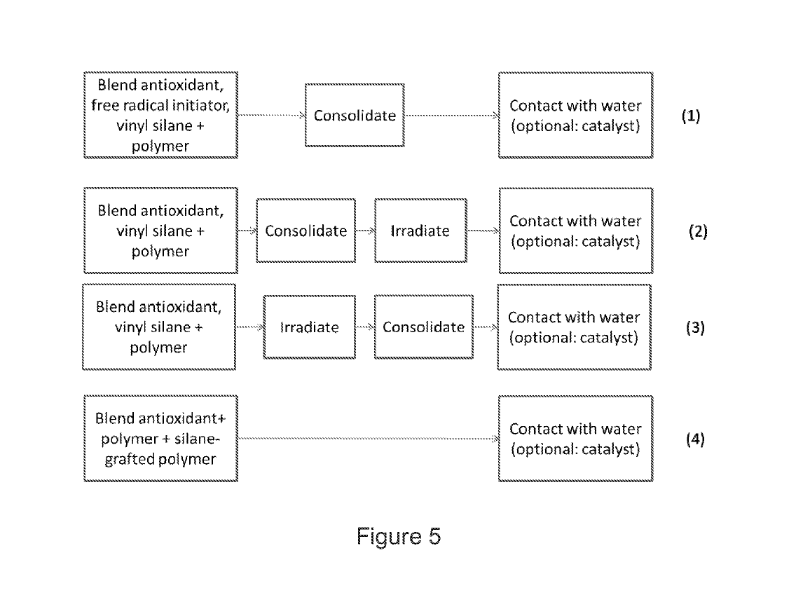

It is known that vinyl silanes can act as cross-linking agents in polymeric materials, specifically polyolefins. Once, free radicals are generated on the polymer backbone, vinyl silanes are grafted onto the polymer. When the silane-grafted polymeric material is contacted with water, or an environment with increased humidity, preferably in the presence of a catalyst, the alkoxysilane groups are converted to hydroxyls (silanols), which can then condense, preferably with the aid of a condensation catalyst into Si-oxygen-Si bonds to cross-link polymer chains (see FIG. 4). Some methods involving the incorporation of antioxidant(s) into silane-crosslinked polymeric material are described schematically in FIG. 5.

In some embodiments, the invention includes methods of making oxidation resistant, substantially cross-linked polymeric material comprising: (a) blending the polymeric material with one or more antioxidant(s), a free radical initiator such as benzoyl peroxide, and one or more vinyl silane(s); (b) consolidating the blend; and (c) contacting the consolidated polymeric material with water in the presence of a catalyst.

In some embodiments, the invention includes methods of making oxidation resistant, substantially cross-linked polymeric material comprising: (a) blending the polymeric material with one or more antioxidant(s), a free radical initiator such as benzoyl peroxide, and one or more vinyl silane(s); (b) consolidating the blend; and (c) contacting the consolidated polymeric material with water in the absence of a catalyst.

In some embodiments, the invention includes methods of making oxidation resistant, substantially cross-linked polymeric material comprising: (a) blending the polymeric material with a free radical initiator such as benzoyl peroxide, and one or more vinyl silane(s); (b) consolidating the blend; (c) doping the consolidated polymeric material by one or more antioxidant(s) by diffusion; and (d) contacting the consolidated polymeric material with water in the presence of a catalyst.

In some embodiments, the invention includes methods of making oxidation resistant, substantially cross-linked polymeric material comprising: (a) blending the polymeric material with a free radical initiator such as benzoyl peroxide, one or more vinyl silane(s); (b) consolidating the blend; (c) doping the consolidated polymeric material by one or more antioxidant(s) by diffusion; and (d) contacting the consolidated polymeric material with water in the absence of a catalyst.

In some embodiments, the invention includes methods of making oxidation resistant, substantially cross-linked polymeric material comprising: (a) blending the polymeric material with a free radical initiator such as benzoyl peroxide, one or more vinyl silane(s); (b) consolidating the blend; (c) contacting the consolidated polymeric material with water in the presence of a catalyst; and (d) doping the consolidated polymeric material by one or more antioxidant(s) by diffusion.

In some embodiments, the invention includes methods of making oxidation resistant, substantially cross-linked polymeric material comprising: (a) blending the polymeric material with a one or more vinyl silane(s); (b) consolidating the blend; (c) irradiating the consolidated blend; (d) contacting the consolidated polymeric material with water in the presence of a catalyst; and (e) doping the consolidated polymeric material by one or more antioxidant(s) by diffusion.

In some embodiments, the invention includes methods of making oxidation resistant, substantially cross-linked polymeric material comprising: (a) blending the polymeric material with a one or more antioxidant(s) and one or more vinyl silane(s); (b) consolidating the blend; (c) irradiating the consolidated blend; and (d) contacting the consolidated polymeric material with water in the presence of a catalyst.

In some embodiments, the invention includes methods of making oxidation resistant, substantially cross-linked polymeric material comprising: (a) blending the polymeric material with one or more antioxidant(s) and one or more vinyl silane(s); (b) consolidating the blend; (c) diffusing one or more free radical initiator(s) into the consolidated blend; (d) heating the consolidated, free radical initiator-doped blend; and (d) contacting with water in the presence of a catalyst.

In some embodiments, the invention includes methods of making oxidation resistant, substantially cross-linked polymeric material comprising: (a) blending the polymeric material with one or more antioxidant(s) and one or more vinyl silane(s); (b) consolidating the blend; (c) diffusing one or more free radical initiator(s) into the consolidated blend; (d) heating the consolidated, free radical initiator-doped blend, thereby obtaining a silane-grafted polymeric material; (e) diffusing a catalyst into the silane grafted polymeric material; and (f) contacting with water.

### Additional Treatments

Several pre- and post-crosslinking treatments may be utilized to improve the oxidation resistance, wear resistance, or mechanical strength of the polymeric material. For example, high pressure crystallization of UHMWPE leads to the formation of a hexagonal crystalline phase and induces higher crystallinity and higher mechanical strength in uncross-linked and cross-linked UHMWPE, more so in the presence of a plasticizing agent such as vitamin E. High pressure crystallization methods are described U.S. Patent Application Publication Nos. 2007/0265369 and 2007/0267030 to Muratoglu et al.

In some embodiments, the antioxidant contained in an article made of polymeric material may be decreased after peroxide diffusion and/or cross-linking. To prevent oxidation on the antioxidant-poor region(s), the cross-linked polymeric material, medical implant preform or medical implant can be treated by using one or more of the following methods:
(1) doping with antioxidant(s) through diffusion at an elevated temperature below or above the melting point of the cross-linked article;
(2) mechanically deforming of the UHMWPE followed by heating below or above the melting point of the article;
(3) high pressure crystallization or high pressure annealing of the article; and
(4) further heat treating the article.
   After one or more of these treatments, the free radicals are stabilized or practically eliminated everywhere in the article.

It may be desirable that after cross-linking, any heat treatments close to or above the melting temperature of the polymeric material not decrease the crystallinity significantly. A decrease in crystallinity may be accompanied by a decrease in mechanical strength, as determined by impact strength, ultimate tensile strength or fatigue strength.

To maintain the crystallinity of the polymeric material, the heat treatments involved in diffusion of the antioxidant(s) and/or the cross-inking agent(s) and the activation of the crosslinking agent(s) can be performed under pressure to elevate the melting temperature of the polymeric material. In this way, melting during or after cross-linking can be avoided and mechanical properties maintained.

In some embodiments, mechanical annealing of cross-linked polymeric material can be performed. General methods for mechanical annealing of uncross-linked and cross-linked polymeric materials, also in the presence of antioxidants and plasticizing agents are described in, for example, U.S. Pat. Nos. 7,166,650 and 7,431,874, and U.S. Patent Application Publication Nos. 2007/0265369 and 2007/0267030, the contents of which are incorporated herein by reference in their entirety. In another embodiment, invention provides methods to improve oxidative stability of polymers by mechanically deforming the irradiated antioxidant-containing polymers to reduce or eliminate the residual free radicals. General mechanical deformation methods have been described in, for example, U.S. Patent Publication Nos. 2004/0156879 and US 2005/0124718; and PCT Patent Application Publication No. WO 2005/074619, the contents of which are incorporated herein by reference in their entirety.

Some embodiments of the present invention also include methods that allow reduction in the concentration of residual free radical in irradiated polymer, even to undetectable levels, without heating the material above its melting point. This method involves subjecting an irradiated sample to a mechanical deformation that is below the melting point of the polymer. The deformation temperature could be as high as about 135° C., for example, for UHMWPE. The deformation causes motion in the crystalline lattice, which permits recombination of free radicals previously trapped in the lattice through cross-linking with adjacent chains or formation of trans-vinylene unsaturations along the back-bone of the same chain. If the deformation is of sufficiently small amplitude, plastic flow can be avoided. The percent crystallinity should not be compromised as a result. Additionally, it is possible to perform the mechanical deformation on machined components without loss in mechanical tolerance. The material resulting from the present invention is a cross-linked polymeric material that has reduced concentration of residuals free radical, and preferably substantially no detectable free radicals, while not substantially compromising the crystallinity and modulus.

Some embodiments of the present invention further provide that the deformation can be of large magnitude, for example, a compression ratio of 2. The deformation can provide enough plastic deformation to mobilize the residual free radicals that are trapped in the crystalline phase. It also can induce orientation in the polymer that can provide anisotropic mechanical properties, which can be useful in implant fabrication. If not desired, the polymer orientation can be removed with an additional step of heating at an increased temperature below or above the melting point.

According to another aspect of the invention, a high strain deformation can be imposed on the irradiated component. In this fashion, free radicals trapped in the crystalline domains likely can react with free radicals in adjacent crystalline planes as the planes pass by each other during the deformation-induced flow. High frequency oscillation, such as ultrasonic frequencies, can be used to cause motion in the crystalline lattice. This deformation can be performed at elevated temperatures that is below the melting point of the polymeric material, and with or without the presence of a sensitizing gas. The energy introduced by the ultrasound yields crystalline plasticity without an increase in overall temperature.

The present invention also provides methods of further heating following free radical elimination below melting point of the polymeric material. According to the invention, elimination of free radicals below the melt is achieved either by the sensitizing gas methods and/or the mechanical deformation methods. Further heating of cross-linked polymer containing reduced or no detectable residual free radicals is done for various reasons, for example:

1. Mechanical deformation, if large in magnitude (for example, a compression ratio of two during channel die deformation), will induce molecular orientation, which may not be desirable for certain applications, for example, acetabular liners. Accordingly, for mechanical deformation:
a) Thermal treatment below the melting point (for example, less than about 137° C. for UHMWPE) is utilized to reduce the amount of orientation and also to reduce some of the thermal stresses that can persist following the mechanical deformation at an elevated temperature and cooling down. Following heating, it is desirable to cool down the polymer at slow enough cooling rate (for example, at about 10° C./hour) so as to minimize thermal stresses. If under a given circumstance, annealing below the melting point is not sufficient to achieve reduction in orientation and/or removal of thermal stresses, one can heat the polymeric material to above its melting point.
b) Thermal treatment above the melting point (for example, more than about 137° C. for UHMWPE) can be utilized to eliminate the crystalline matter and allow the polymeric chains to relax to a low energy, high entropy state. This relaxation leads to the reduction of orientation in the polymer and substantially reduces thermal stresses. Cooling down to room temperature is then carried out at a slow enough cooling rate (for example, at about 10° C./hour) so as to minimize thermal stresses.

2. The contact before, during, and/or after irradiation with a sensitizing environment to yield a polymeric material with no substantial reduction in its crystallinity when compared to the reduction in crystallinity that otherwise occurs following irradiation and subsequent or concurrent melting. The crystallinity of polymeric material contacted with a sensitizing environment and the crystallinity of radiation treated polymeric material is reduced by heating the polymer above the melting point (for example, more than about 137° C. for UHMWPE). Cooling down to room temperature (about 20° C. to 25° C.) is then carried out at a slow enough cooling rate (for example, at about 10° C./hour) so as to minimize thermal stresses.

As described herein, it is demonstrated that mechanical deformation can eliminate residual free radicals in a radiation cross-linked UHMWPE. The invention also provides that one can first deform UHMWPE to a new shape either at solid- or at molten-state, for example, by compression. According to a process of the invention, mechanical deformation of UHMWPE when conducted at a molten-state, the polymer is crystallized under load to maintain the new deformed shape. Following the deformation step, the deformed UHMWPE sample is irradiated below the melting point to cross-link, which generates residual free radicals. To eliminate these free radicals, the irradiated polymer specimen is heated to a temperature below the melting point of the deformed and irradiated polymeric material (for example, up to about 135° C. for UHMWPE) to allow for the shape memory to partially recover the original shape. Generally, it is expected to recover about 80-90% of the original shape. During this recovery, the crystals undergo motion, which can help the free radical recombination and elimination. The above process is termed as a “reverse-IBMA”. The reverse-IBMA (reverse-irradiation below the melt and mechanical annealing) technology can be a suitable process in terms of bringing the technology to large-scale production of UHMWPE-based medical devices.

The consolidated polymeric materials according to any of the methods described herein can be irradiated at room temperature or at an elevated temperature below or above the melting point of the polymeric material.

In one embodiment, the polymeric material can be mechanically deformed at any processing step during peroxide cross-linking. For example, polymeric material can be blended with one or more antioxidant(s). The blend can be consolidated into implant preform shape. The implant preform can be mechanically deformed at any temperature, preferably an elevated temperature below the melting point of the polymeric material. Then, the deformed antioxidant blended polymeric material is diffused with one or more cross-linking agent(s). At least one cross-linking agent can be a peroxide. The antioxidant blended and cross-linking agent diffused polymeric material can be heated for a period of time. Then the implant preform can be machined into final implant shape. The final implant is packaged and sterilized.

In certain embodiments of the present invention any of the method steps disclosed herein, including blending, mixing, consolidating, quenching, irradiating, annealing, mechanically deforming, doping, homogenizing, heating, melting, and packaging of the finished product, such as a medical implant, can be carried out in presence of a sensitizing gas and/or liquid or a mixture thereof, inert gas, air, vacuum, and/or a supercritical fluid.

In some embodiments, high temperature melting of polymeric material can be used to improve the impact toughness of the polymeric material and its blends with antioxidant(s) and/or cross-linking agent(s). In some embodiments, the polymeric material is blended with one or more antioxidant(s). The polymeric blend is consolidated into an implant preform. Then, one or more crosslinking agent(s) are diffused into the consolidated blend by immersing the blend in the pure crosslinking agents or a solution of the crosslinking agent(s). The cross-linking agent can be chosen from peroxides. The implant preform is heated to an elevated temperature above the melting point, for example 300° C. in inert atmosphere. The implant is maintained at temperature for a duration between 1 minute to 24 hours, more preferably from 1 hour to 10 hours, most preferably about 5 hours. The implant preform is machined to obtain a final implant before and after diffusion of the cross-linking agent, or before or after high temperature melting. The final implant is packaged and sterilized by irradiation or gas sterilization. The implant preform or implant can be irradiated before or after the diffusion of the cross-linking agent, or before or after high temperature melting.

In some embodiments, high temperature melting can be performed at any step during manufacturing of the polymeric material or implant preform or implant. In some embodiments, high temperature melting can be used to enhance mechanical properties and in some embodiments, it can be used simply as a heat treatment, for example to reduce free radicals or to initiate the decomposition of a cross-linking agent or peroxide for cross-linking the polymeric material.

In some embodiments, high temperature melting of polymeric material can be used to improve the impact toughness of the polymeric material and its blends with antioxidant(s) and/or cross-linking agent(s). In some embodiments, the polymeric material is blended with one or more antioxidant(s). The polymeric blend is consolidated into an implant preform. The implant preform is heated to an elevated temperature above the melting point, for example 300° C. in inert atmosphere. The implant preform is maintained at temperature for a duration between 1 minute to 24 hours, more preferably from 1 hour to 10 hours, most preferably about 5 hours. The implant preform is cooled at any rate, for example 2° C./min or below. Then, one or more crosslinking agent(s) are diffused into the high temperature melted implant preform by immersing it in the pure crosslinking agents or a solution of the crosslinking agent(s). The cross-linking agent can be chosen from peroxides. Then the diffused implant preform can be heated to above the decomposition temperature of the peroxide(s) to cross-link (further) the diffused implant preform. The cross-linked implant preform is machined to obtain a final implant before and after diffusion of the cross-linking agent, or before or after high temperature melting. The final implant is packaged and sterilized by irradiation or gas sterilization. The implant preform or implant can be irradiated before or after the diffusion of the cross-linking agent, or before or after high temperature melting.

In some embodiments, the polymeric material is blended with one or more antioxidant(s). The polymeric blend is consolidated into an implant preform. The implant preform is cooled at any rate, for example 2° C./min or below. Then, one or more crosslinking agent(s) are diffused into the high temperature melted implant preform by immersing it in the pure crosslinking agents or a solution of the crosslinking agent(s) close to or below the decomposition temperature(s) of the cross-linking agent(s). The cross-linking agent can be chosen from peroxides. The implant preform is heated to an elevated temperature above the melting point, for example 300° C. in inert atmosphere to decompose the cross-linking agent(s). The implant preform is maintained at temperature for a duration between 1 minute to 24 hours, more preferably from 1 hour to 10 hours, most preferably about 5 hours. The cross-linked implant preform is machined to obtain a final implant before and after diffusion of the cross-linking agent, or before or after high temperature melting. The final implant is packaged and sterilized by irradiation or gas sterilization. The implant preform or implant can be irradiated before or after the diffusion of the cross-linking agent, or before or after high temperature melting.

In another embodiment, invention provides methods to improve oxidative stability of polymers by diffusing more antioxidant into the irradiated polymer-antioxidant blend. Antioxidant diffusion methods have been described, for example, in U.S. Patent Application Publication Nos. 2004/0156879 and 2008/0214692 and PCT Patent Application Publication No. WO 2007/024689, the contents of which are incorporated herein by reference in their entirety.

High temperature melting methods have been described by Oral et al. in PCT Patent Application Publication No. WO 2010/096771, which is incorporated herein as reference.

### Doping/Diffusion Of Additives

Diffusion and penetration depth in irradiated UHMWPE has been discussed. Muratoglu et al. (see U.S. Patent Application Publication No. 2004/0156879) described, among other things, high temperature doping and/or annealing steps to increase the depth of penetration of α-tocopherol into radiation cross-linked UHMWPE. Muratoglu et al. (see U.S. Patent Application Publication No. 2008/0214692) described annealing in supercritical carbon dioxide to increase depth of penetration of α-tocopherol into irradiated UHMWPE.

Doping of the polymeric material with an additive such as a cross-linking agent or an antioxidant can be done through diffusion at a temperature above the melting point of the irradiated polymeric material (for example, at a temperature above 137° C. for UHMWPE) can be carried out under sub-ambient pressure, ambient pressure, elevated pressure, and/or in a sealed chamber. Doping above the melting point can be done by soaking the article in vitamin Eat a temperature above 137° C. for at least 10 seconds to about 100 hours or longer. At elevated pressures, the melting point of polymeric material can be elevated, therefore temperature ranges ‘below’ and ‘above’ the melting point may change under pressure.

Polymeric material can be doped with an antioxidant by soaking the material in the additive, a mixture of additives or a solution of the additive. This allows the additive to diffuse into the polymer. For instance, the material can be soaked in 100% peroxide. The material also can be soaked in a cross-linking agent solution where a carrier solvent can be used to dilute the cross-linking agent concentration. To increase the depth of diffusion, the material can be doped for longer durations, at higher temperatures, at higher pressures, and/or in presence of a supercritical fluid. The additive can be diffused to a depth of about 5 millimeters or more from the surface, for example, to a depth of about 3-5 millimeters, about 1-3 millimeters, or to any depth thereabout or therebetween.

The doping process can involve soaking of a polymeric material, medical implant or device with an additive, such as a peroxide, for about half an hour up to several days, preferably for about one hour to 24 hours, more preferably for one hour to 16 hours. The additive or additive solution can be at room temperature or heated up to about 137° C. and the doping can be carried out at room temperature or at a temperature up to about 137° C. The additive solution can be below, at or above the decomposition temperature of the peroxide(s) being used. Preferably the additive solution is heated to a temperature between about 60° C. and 120° C., or about 100° C. and 135° C. or between about 110° C. and 130° C., and the doping is carried out at a temperature between about 60° C. and 135° C. or between about 60° C. and 100° C.

Doping with additive(s) through diffusion at a temperature above the melting point of the irradiated polyethylene (for example, at a temperature above 137° C.) can be carried out under reduced pressure, ambient pressure, elevated pressure, and/or in a sealed chamber, for about 0.1 hours up to several days, preferably for about 0.5 hours to 6 hours or more, more preferably for about 1 hour to 5 hours. The additives or additive solution can be at a temperature of about 137° C. to about 400° C., more preferably about 137° C. to about 200° C., more preferably about 137° C. to about 160° C.

The doping and/or the irradiation steps can be followed by an additional step of “homogenization”, which refers to a heating step in air or in anoxic environment to improve the spatial uniformity of the additive concentration within the polymeric material, medical implant or device. Homogenization also can be carried out after any doping step. The heating may be carried out above or below or at the peak melting point. Additive-doped or -blended polymeric material can be homogenized at a temperature below or above or at the peak melting point of the polymeric material for a desired period of time, for example, the peroxide-doped polymeric material can be homogenized for about an hour to several days at room temperature to about 100° C. In the case of peroxide-doped polymeric material, homogenization can be carried out below, close to or above the decomposition temperature. Preferably, homogenization is close to or below the decomposition temperature to diffuse the peroxide(s) without substantially decomposing them. Preferably, the homogenization is carried out at 0° C. to 400° C., or at 30° C. to 120° C. or at 90° C. to 180° C., more preferably 80° C. to 100° C. Homogenization is preferably carried out for about one minute to several months, one hour to several days to two weeks or more, more preferably about 1 hour to 24 hours or more, more preferably about 4 hours. More preferably, the homogenization is carried out at about 100° C. for about 4 hours or at about 120° C. for about 4 hours. The polymeric material, medical implant or device is kept in an inert atmosphere (nitrogen, argon, and/or the like), under vacuum, or in air during the homogenization process. The homogenization also can be performed in a chamber with supercritical fluids such as carbon dioxide or the like. The pressure of the supercritical fluid can be about 1000 to about 3000 psi or more, more preferably about 1500 psi. It is also known that pressurization increases the melting point of UHMWPE. A higher temperature than 137° C. can be used for homogenization below the melting point if applied pressure has increased the melting point of UHMWPE.

The terms “extraction” or “elution” from consolidated polymeric material refers to partial or complete removal of absorbed components, for example peroxide decomposition products, from the consolidated polymeric material by various processes disclosed herein. For example, the extraction or elution can be done with a compatible solvent that dissolves the components contained in the consolidated polymeric material. Such solvents include, but not limited to, a hydrophobic solvent, such as hexane, heptane, or a longer chain alkane; an alcohol such as ethanol, any member of the propanol or butanol family or a longer chain alcohol; or an aqueous solution in which the components, such as peroxide decomposition products are soluble. Such a solvent also can be made by using an emulsifying agent such as Tween 80 or ethanol.

Extraction of components from polymeric material at a temperature below the melting temperature of the polyethylene can be achieved by placing the polymeric material in an open or in a sealed chamber. A solvent or an aqueous solution also can be added in order to extract the extractable components from the polymeric material. The chamber is then heated below the melting point of the polymeric material, preferably between about room temperature to near the melting point, more preferably about 100° C. to about 137° C., more preferably about 120° C., or more preferably about 130° C. If a sealed chamber is used, there will be an increase in pressure during heating. Because the polyethylene is cross-linked, only the crystalline regions melt. The chemical cross-links between chains remain intact and allow the polyethylene to maintain its shape throughout the process despite surpassing its melting temperature. Increasing pressure increases the melting temperature of the polymeric material. In this case, homogenization below the melt is performed under pressure above 137° C., for example at about 145° C.

Extraction of components from a polyethylene at a temperature above the melting temperature of the polyethylene can be achieved by placing the polyethylene in an open or in a sealed chamber. A solvent or an aqueous solution also can be added in order to extract the components from polyethylene. The chamber is then heated above the melting point of the polyethylene, preferably between about 137° C. to about 400° C., more preferably about 137° C. to about 200° C., more preferably about 137° C., or more preferably about 160° C. If a sealed chamber is used, there will be an increase in pressure during heating. Because the polyethylene is cross-linked, only the crystalline regions melt. The chemical cross-links between chains remain intact and allow the polyethylene to maintain its shape throughout the process despite surpassing its melting temperature. Since crystallites pose a hindrance to diffusion of additives in polyethylene, increasing the temperature above the melting point should increase the rate of extraction of components. Increasing pressure increases the melting temperature of the polymeric material.

To prevent oxidation any cross-linked polymeric material, it can be treated by using one or more of the following methods:
(1) doping with an antioxidant through diffusion at an elevated temperature below or above the melting point of the cross-linked polymeric material;
(2) melting;
(3) mechanically deforming the polymeric material followed by heating below or above the melting point of the polymeric material; and
(4) high pressure crystallization or high pressure annealing of the polymeric material.

Polyethylene undergoes a phase transformation at elevated temperatures and pressures from the orthorhombic to the hexagonal crystalline phase. The hexagonal phase can grow extended chain crystals and result in higher crystallinity in polyethylene. This is believed to be a consequence of less hindered crystallization kinetics in the hexagonal phase compared with the orthorhombic phase. High pressure crystallization can be achieved with one of two methods:
A. Route I: Heat to the desired temperature, for example, above the melt (for example, about 140° C., about 180° C., about 200° C., about 250° C., or about 300° C.); then pressurize; then hold pressure at about the same pressure, for one minute to a day or more, preferably about 0.5 hours to 12 hours, more preferably 1 to 6 hours; then release the pressure (pressure has to be released after cooling down to room temperature to avoid melting of the crystals achieved under high pressure).
B. Route II: Pressurize to the desired pressure; then heat to the desired temperature, for example, below the melt of pressurized polyethylene (for example, about 150° C., about 180° C., about 195° C., about 225° C., about 300° C., and about 320° C.); then hold pressure at about the same pressure, for one minute to a day or more, preferably about 0.5 hours to 12 hours, more preferably 1 to 6 hours; then cool to room temperature; then release the pressure (pressure has to be released after cooling down to room temperature to avoid melting of the crystals achieved under high pressure).

Manufacturing methods. An additive-blended polymer such as vitamin E-blended UHMWPE can be cross-linked by using cross-linking agents during consolidation. In the case of UHMWPE, the consolidation can be most commonly performed by hot isostatic pressing (HIPping), compression molding or direct compression molding (FIG. 2). HIPping or compression molding results commonly in large bar stock from which the desired shaped can be finalized by types of machining. While direct compression molding is intended commonly to result in a final-shape implant, a small amount of machining can follow the direct compression molding step to convert the near-net shape implant preform to final-shape. The final step in manufacturing is appropriate packaging and terminal sterilization. Terminal sterilization can be an irradiation method, or a non-irradiation method such as ethylene oxide or gas plasma sterilization. It could also be a method in which the material is exposed to any environment that can reduce the amount of bacteria or external agents to levels specified by sterility requirements for s desired application. Such a method can include exposure to supercritical fluid(s).

These steps in the manufacturing scheme are not limiting, that is, additional process steps can be interjected. For example, additional chemical cross-linking, irradiation or heat processing can be done after the consolidation before or after machining. Alternatively, additional antioxidant stabilization can be achieved by introducing more antioxidant by diffusion after the consolidation processes before or after machining.

The invention is further illustrated in the following Examples which are presented for purposes of illustration and not of limitation.

### EXAMPLES

### Example 1

### Preparation of Blends

Vitamin E was blended with UHMWPE powder with the aid of isopropyl alcohol (IPA). Vitamin E was dissolved in IPA to prepare a vitamin E solution. The vitamin E solution was added to the UHMWPE powder in a closed container that was subjected to vigorous shaking to prepare the vitamin E/UHMWPE blend. Subsequently, the IPA was evaporated out of the vitamin E/UHMWPE blend at room temperature. Vitamin E/UHMWPE blends with various concentrations were prepared and used in the following examples. Unless otherwise noted, all vitamin E/UHMWPE blends used in the following examples were fabricated using this Example 1.

Vitamin E is blended with polymeric material with the aid of a solvent. Vitamin E is dissolved in the solvent to prepare a vitamin E solution. The vitamin E solution is added to the polymeric material in a closed container that is subjected to vigorous shaking to prepare the vitamin E/polymeric material blend. Subsequently, the solvent is evaporated out of the vitamin E/polymeric material blend.

Antioxidant(s) is blended with polymeric material with the aid of a solvent. Antioxidant(s) is dissolved in the solvent to prepare an antioxidant(s) solution. The antioxidant(s) solution is added to the polymeric material in a closed container that is subjected to vigorous shaking to prepare the antioxidant(s)/polymeric material blend. Subsequently, the solvent is evaporated out of the antioxidant(s)/polymeric material blend.

In the examples below the antioxidant/polymeric material blend (such as vitamin E/UHMWPE blend) is mixed with different cross-linking agents such as peroxides. The mixing of the antioxidant(s)/polymeric material blend and the cross-linking agent(s) is done in a closed container. The container is subjected to vigorous shaking. For example the shaking is done using a commercial Turbula TF2 Shaker-Mixer.

In the examples below the geometries of the samples used are optionally interchanged with the shape of an implant, or the shape of an implant preform, or a stock large enough to be able to machine an implant at any step of processing.

### Example 2

### Chemical Cross-Linking of Antioxidant-Containing Polymeric Material with High Pre-Heat Temperature (Vitamin E as Model Antioxidant)

Vitamin E/UHMWPE blend with 0.1 wt % vitamin E was used. Then the chosen peroxides (Table 2; DCP, BP and Luperox®-130) were each blended with vitamin E-UHMWPE blend by direct mixing (Luperox®-130) or with the aid of a solvent such as IPA (DCP) or acetone (BP). Luperox-130 is liquid at room temperature; therefore it was directly mixed with the vitamin E/UHMWPE blend in a closed container and was subjected to vigorous shaking by hand. DCP is solid at room temperature; therefore it was dissolved in IPA to form a DCP solution. The DCP solution was then mixed with the vitamin E/UHMWPE blend in a closed container and subsequently was subjected to vigorous shaking by hand. Similarly, the BP is solid at room temperature; therefore it was dissolved in acetone to form a BP solution. The BP solution was then mixed with the vitamin E/UHMWPE blend in a closed container and subsequently was subjected to vigorous shaking by hand. The concentration of the peroxide in all three groups of blends was 1 wt %. In the latter two blends, the solvents were substantially removed from the polymer blend by evaporation at ambient pressure at close to room temperature. The vigorous shaking of the blends mentioned in this example could also be done by shaking the containers using the Turbula TF2.

TABLE 2
Some Examples Of Peroxides That Are Used In Peroxide Containing UHMWPE.

| | | 1 hour | 10 hour |
|---|---|---|---|
| | | half-life | half-life |
| | | temperature | temperature |
| Chemical Structure | Peroxide Name | (T₁) (° C.) | (T₁₀)(° C.) |
| 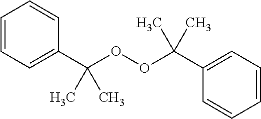 [SMILES: CC(C)(OOC(C)(C)c1ccccc1)c1ccccc1] | Dicumyl peroxide (in benzene) (DCP) | 137 | 117 |
| 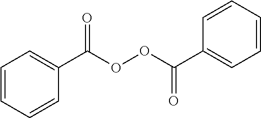 [SMILES: O=C(OOC(=O)c1ccccc1)c1ccccc1] | Benzoyl peroxide (in benzene) (BP) (Also known as dibenzoyl peroxide) | 91 | 73 |
| 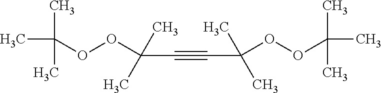 [SMILES: CC(C)(C)OOC(C)(C)C#CC(C)(C)OOC(C)(C)C] | 2,5-dimethyl-2,5-Di-(t- butylperoxy)hexyne-3 (Luperox ®-130) (in dodecane) | 152 | 131 |
| 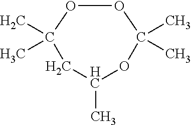 | 3,3,5,7,7-pentamethyl 1,2,4-trioxepane (Trigonox ® 311) | 184 | 158 |

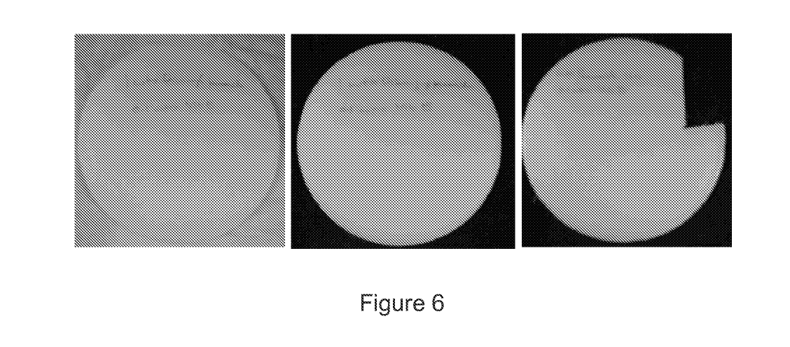

The peroxide and vitamin E blended UHMWPEs were pre-heated in a mold at about 195-200° C. in inert gas for about 1 hour. Then they were consolidated into pucks (diameter 10 cm, thickness 1 cm; see FIG. 6) with the press platens at 181° C. and 20 MPa for 5 minutes with a cool-down to room temperature of about 45 minutes.

Subsequently the consolidated blends are optionally heated to above the dissociation temperature of the peroxide used to further cross-link the polymer. Another optional step is the extraction of the unreacted peroxides and their byproducts from the polymer after consolidation and/or after the subsequent heating step.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before or after the final heating step or before or after the optional extraction step.

### Example 3

### Chemical Cross-Linking of Antioxidant-Containing Polymeric Material with Low Pre-Heat Temperature (Vitamin E as Antioxidant)

Luperox®-130 (see Table 2) is blended with vitamin E/UHMWPE blend with 0.5 wt % vitamin E using the Turbula TF2. The concentration of the peroxide in the blend is 2 wt %.

The peroxide and vitamin E blended UHMWPE is pre-heated in a mold at about 135° C. in inert gas for about 1 hour. Then it is transferred to between press platens at about 180° C. and the mold is closed and contacted with the heated platens from both sides for about 10 minutes. Then it is consolidated into a puck (diameter 10 cm, thickness 1 cm) with the press platens at about 180° C. and under a pressure of about 20 MPa for about 5 minutes with a cool-down to room temperature of about 45 minutes.

Subsequently the consolidated blends are optionally heated to above the peroxide initiation temperature to further cross-link the polymer. Another optional step is the extraction of the unreacted peroxides and their byproducts from the polymer after consolidation and/or after the subsequent heating step.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before or after the final heating step or before or after the optional extraction step.

### Example 4

### Chemical Cross-Linking of Antioxidant-Containing Polymeric Material with No Pre-Heat (Vitamin E as Antioxidant)

Luperox®-130 (Table 2) is blended with vitamin E/UHMWPE containing 0.5 wt % vitamin E by mixing using Turbula TF2. The concentration of the peroxide in the blend is 2 wt %.

The peroxide and vitamin E blended UHMWPE is assembled in a mold at room temperature. Then it is transferred to between press platens at 180° C. and the mold is closed and contacted with the heated platens from both sides for 10 minutes. Then it is consolidated into a puck (diameter 10 cm, thickness 1 cm) with the press platens at 185° C. and under a pressure of 20 MPa for 5 minutes with a cool-down to room temperature of about 45 minutes.

Subsequently the consolidated blends are optionally heated to above the peroxide initiation temperature to further cross-link the polymer. Another optional step is the extraction of the unreacted peroxides and their byproducts from the polymer after consolidation and/or after the subsequent heating step.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before or after the final heating step or before or after the optional extraction step.

### Example 5

### Chemical Cross-Linking of Antioxidant-Containing Polymeric Material with Low Pre-Heat Temperature with Different Peroxide Concentrations (Vitamin E as Antioxidant)

Benzoyl peroxide (BP; Table 2) is dissolved in acetone and the resulting solution is blended with vitamin E/polymeric material blend by mixing using the Turbula TF2. The polymeric material is optionally UHMWPE. The concentration of the vitamin E in vitamin E/polymeric material blend is 0.1 wt %, 0.2 wt %, 0.5 wt %, 0.6 wt %, 0.7 wt %, 0.8 wt %, 0.9 wt %, 1 wt %, or more. The concentration of the peroxide in the blend is 0.1 wt %, 0.5 wt %, 0.7 wt %, 1 wt %, 1.5 wt %, 2 wt %, 2.5 wt % or 3 wt %. The acetone is substantially removed from the polymer blend by evaporation at ambient pressure at close to room temperature.

The BP and vitamin E blended polymeric material is pre-heated in a mold at 70° C. in inert gas for about 1 hour. Then it is transferred to between press platens at 180° C. and the mold is closed and contacted from both sides with the heated platens for 10 minutes. Then it is consolidated into a puck (diameter 10 cm, thickness 1 cm) with the press platens at 180° C. and under a pressure of 20 MPa for 5 minutes with a cool-down to room temperature of about 45 minutes.

DCP (Table 2) is dissolved in IPA and the resulting solution is blended with vitamin E/polymeric material by mixing using the Turbula TF2. The concentration of the vitamin E in vitamin E/polymeric material blend is 0.1 wt %, 0.2 wt %, 0.5 wt %, 0.6 wt %, 0.7 wt %, 0.8 wt %, 0.9 wt %, 1 wt %, or more. The polymeric material is optionally UHMWPE. The concentration of the peroxide in the blend is 0.1 wt %, 0.5 wt %, 0.7 wt %, 1 wt %, 1.5 wt %, 2 wt %, 2.5 wt % or 3 wt %. The IPA is substantially removed from the polymer blend by evaporation at ambient pressure at close to room temperature.

The DCP and vitamin E blended polymeric material is pre-heated in a mold at 115° C. in inert gas for about 1 hour. Then it is transferred to between press platens at 180° C. and the mold is closed and contacted from both sides with the heated platens for 10 minutes. Then it is consolidated into a puck (diameter 10 cm, thickness 1 cm) with the press platens at 180° C. and under a pressure of 20 MPa for 5 minutes with a cool-down to room temperature of about 45 minutes.

Luperox®-130 (P130; Table 2) is blended with vitamin E/polymeric material blend by mixing using the Turbula TF2. The concentration of the vitamin E in vitamin E/polymeric material blend is 0.1 wt %, 0.2 wt %, 0.5 wt %, 0.6 wt %, 0.7 wt %, 0.8 wt %, 0.9 wt %, 1 wt %, or more. The polymeric material is optionally UHMWPE. The concentration of the peroxide in the blend is 0.1 wt %, 0.5 wt %, 0.7 wt %, 1 wt %, 1.5 wt %, 2 wt %, 2.5 wt % or 3 wt %.

The L-130 and vitamin E blended polymeric material is pre-heated in a mold at 135° C. in inert gas for about 1 hour. Then it is transferred to between press platens at 180° C. and the mold is closed and contacted from both sides with the heated platens for 10 minutes. Then it is consolidated into a puck (diameter 10 cm, thickness 1 cm) with the press platens at 180° C. and under pressure of 20 MPa for 5 minutes with a cool-down to room temperature of about 45 minutes.

Trigonox® 311 (T311, Table 2) is blended with vitamin E/polymeric material blend by mixing using the Turbula TF2. The concentration of the vitamin E in vitamin E/polymeric material blend is 0.1 wt %, 0.2 wt %, 0.5 wt %, 0.6 wt %, 0.7 wt %, 0.8 wt %, 0.9 wt %, 1 wt %, or more. The polymeric material is optionally UHMWPE. The concentration of the peroxide in the blend is 0.1 wt %, 0.5 wt %, 0.7 wt %, 1 wt %, 1.5 wt %, 2 wt %, 2.5 wt % or 3 wt %.

The T-311 and vitamin E blended polymeric material is pre-heated in a mold at 150° C. in inert gas for about 1 hour. Then it is transferred to between press platens at 180° C. and the mold is closed and contacted from both sides with the heated platens for 10 minutes. Then it is consolidated into a puck (diameter 10 cm, thickness 1 cm) with the press platens at 180° C. and under pressure of 20 MPa for 5 minutes with a cool-down to room temperature of about 45 minutes.

Subsequently the consolidated blends are optionally heated to above the peroxide initiation temperature to further cross-link the polymer. Another optional step is the extraction of the unreacted peroxides and their byproducts from the polymer after consolidation and/or after the subsequent heating step.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before or after the final heating step or before or after the optional extraction step.

### Example 6

### Chemical Cross-Linking of Antioxidant-Containing Polymeric Material by Blending, Consolidation and Thermal Treatment (T₁ Close to Molding Temperature) (Vitamin E as Antioxidant)

Vitamin E/polymeric material blends are prepared. Optionally the polymeric material is UHMWPE. The concentration of vitamin E in the polymeric material is 0 wt %, 0.001 wt %, 0.01 wt %, 0.1 wt %, 0.2 wt %, 0.5 wt %, 0.7 wt %, 1 wt %, 10 wt % or more. Then the chosen peroxide (Luperox®-130) is blended with vitamin E/polymeric material blend by mixing using Turbula TF2. The concentration of the peroxide in the blend is 0.1 wt %, 0.5 wt %, 0.8 wt %, 1 wt %, 1.2 wt %, 1.5 wt %, 2 wt % and 5 wt %.

The L-130 and vitamin E blended polymeric material is pre-heated in a mold at 135° C. in inert gas for about 1 hour. Then it is transferred to between press platens and the mold is closed and contacted with the heated platens for 10 minutes. The peroxide and vitamin E blended polymeric material is consolidated into an implant preform at 170° C. or 180° or 190° C. In this case, the molding temperature is above the 1-hour decomposition temperature of the peroxide (T₁=152° C.). The consolidation into a puck (diameter 10 cm, thickness 1 cm) under a pressure of 20 MPa is done in 5 minutes with a cool-down to room temperature of about 45 minutes.

After consolidation, the preform is optionally heated to 180° C., 190° C., 200° C., 210° C., 220° C., 230° C., 240° C., 250° C., 260° C., 270° C., 280° C., 290° C. or 300° C. in air or in inert gas such as nitrogen gas for further cross-linking.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before the final heating step or after the final heating step.

The cross-linked implant preform is machined into an implant. The implant is packaged and sterilized in air or inert atmosphere using gamma irradiation.

### Example 7

### Chemical Cross-Linking of Antioxidant-Containing Polymeric Material by Blending, Consolidation and Thermal Treatment (T₁ Above the Molding Temperature) (Vitamin E as Antioxidant)

Vitamin E/polymeric material blends are prepared. The polymeric material is optionally UHMWPE. The concentration of vitamin E in the polymeric material is 0 wt %, 0.001 wt %, 0.01 wt %, 0.1 wt %, 0.2 wt %, 0.5 wt %, 0.7 wt %, 1 wt %, 10 wt % or more. Then the chosen peroxide (Table 2; Trigonox® 311) is blended with vitamin E/polymeric material blend by mixing using Turbula TF2. Optionally the peroxide is dissolved in a solvent such as acetone and the resulting peroxide solution is mixed with the vitamin E/polymeric material blend using Turbula TF2. The concentration of the peroxide in the blend is 0.1 wt %, 0.5 wt %, 0.8 wt %, 1 wt %, 1.2 wt %, 1.5 wt %, 2 wt % and 10 wt %. The solvent is substantially removed from the polymer blend by evaporation at ambient pressure or vacuum.

The T-311 and vitamin E blended polymeric material is pre-heated in a mold at 135° C. in inert gas for about 1 hour. Then it is transferred to between press platens and the mold is closed and contacted from both sides (top and bottom) with the heated platens for 10 minutes. The peroxide and vitamin E blended UHMWPE is consolidated into an implant preform at 170° C. or 180° C. or 190° C. In this case, the molding temperature is close to the 1-hour decomposition temperature of the peroxide (T₁=184° C.). The consolidation at about 20 MPa of pressure is completed in about 5 minutes with a cool-down to room temperature of about 45 minutes.

After consolidation, the preform is heated to 180° C., 190° C., 200° C., 210° C., 220° C., 230° C., 240° C., 250° C., 260° C., 270° C., 280° C., 290° C. or 300° C. in air or in inert gas such as nitrogen for further cross-linking.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before the final heating step or after the final heating step.

The cross-linked implant preform is machined into an implant. The implant is packaged and sterilized in inert atmosphere using gamma irradiation.

### Example 8

### Additional Antioxidant Stabilization of Peroxide-Crosslinked Polymeric Material (Vitamin E as Antioxidant)

Vitamin E is blended with polymeric material with or without the aid of a solvent such as isopropyl alcohol (IPA) as described in Example 1. The solvent is substantially removed from the polymer blend by evaporation at ambient pressure or vacuum. The concentration of vitamin E in the polymeric material is 0 wt %, 0.001 wt %, 0.01 wt %, 0.1 wt %, 0.2 wt %, 0.5 wt %, 1 wt % and 10 wt %. Then the chosen peroxide (Table 2; Luperox® 130 liquid at room temperature) is blended with vitamin E-blended polymeric material by shaking on the Turbula TF2 Shaker-Mixer. The concentration of the peroxide in the blend is 0.1 wt %, 0.5 wt %, 1 wt %, 2 wt % and 10 wt %.

The L-130 and vitamin E blended polymeric material is pre-heated in a mold at 135° C. in inert gas for about 1 hour. Then it is transferred to between press platens and the mold is closed and contacted from both top and bottom with the heated platens for about 10 minutes. The peroxide and vitamin E blended polymeric material is then consolidated into an implant preform at 170° C. or 180° C. or 190° C. under a pressure of about 20 MPa in about 5 minutes with a cool-down to room temperature of about 45 minutes.

After consolidation, the preform is immersed in vitamin Eat 120° C., 130° C., 140° C., 150° C., 160° C., 170° C., 180° C., 190° C., 200° C., 210° C., 220° C., 230° C., 240° C., 250° C., 260° C., 270° C., 280° C., 290° C. or 300° C. in air or in nitrogen for 1 hour or 5 hours.

The peroxide/UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-cross-linked UHMWPE/vitamin E blend, whereby the irradiation takes place before the final heating step or after the final heating step.

The cross-linked and vitamin E-stabilized implant preform is machined into an implant. The implant is packaged and sterilized in inert atmosphere using gamma irradiation.

### Example 9

### Cross-Linking of UHMWPE by Diffusion of Peroxides into Antioxidant-Containing UHMWPEs Followed by Heating (1) (Vitamin E as Antioxidant)

Vitamin E was blended with UHMWPE powder with the aid of isopropyl alcohol (IPA) as described in Example 1. The solvent was substantially removed from the polymer blend by evaporation at ambient pressure. The concentration of vitamin E in the polymeric material was 0 wt %, 0.1 wt % or 1.0 wt %. The virgin UHMWPE and the vitamin E/UHMWPE blends were consolidated by placing in a mold and pre-heating at 180-190° C. in inert gas for about 1 hour. Then they were consolidated into pucks (diameter 10 cm, thickness 1 cm) with the press platens at 181° C. and under a pressure of about 20 MPa for about 5 minutes with a cool-down to room temperature of about 45 minutes. The pucks were then machined into cubes (1×1×1 cm).

The cubes (n=3) were doped in pre-heated dicumyl peroxide (DCP) at 60° C. for 4 hours. The excess DCP was wiped off the surface of the cubes before cooling down below the solidification temperature of DCP at around 40° C. The cubes were then placed in a pre-heated glass container under inert gas flow at 135° C. and maintained at temperature for 2 hours.

The cross-link density of the surface was measured using small sections close to the surface (approximately 0.5 mm×3×3 mm, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density of virgin, DCP-diffused and heated UHMWPE was 179±74 mol/m³. The cross-link density of 0.1 wt % vitamin E-blended, DCP-diffused and heated UHMWPE was 106±26 mol/m³. The cross-link density of 1.0 wt % vitamin E-blended, DCP-diffused and heated UHMWPE was 61±12 mol/m³. Thus, we demonstrated that cross-linking of UHMWPE without additives and blended with the antioxidant vitamin E could be achieved by diffusing DCP at a temperature below its T₁₀ (approximately 117° C. in this case) into UHMWPE/vitamin E blend and heating the DCP-diffused UHMWPE/vitamin E blend above the T₁₀ of DCP for enough time to allow decomposition of the peroxide. We also demonstrated that cross-link density can be modulated by changing vitamin E concentration in the blend.

The peroxide-diffused UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated cube.

### Example 10

### Cross-Linking of UHMWPE by Diffusion of Peroxides into Antioxidant-Containing UHMWPEs at High Temperature (Vitamin E as Antioxidant)

Vitamin E was blended with UHMWPE powder with the aid of isopropyl alcohol (IPA) as described in Example 1. The solvent was substantially removed from the polymer blend by evaporation at ambient pressure or vacuum. The concentration of vitamin E in the polymeric material was 0 wt % or 0.1 wt %. The virgin or blended UHMWPE powders were placed in a mold and were pre-heated at 180-190° C. in inert gas for about 1 hour. Then they were consolidated into pucks (diameter 10 cm, thickness 1 cm) with the press platens at 181° C. under a pressure of about 20 MPa for about 5 minutes with a cool-down to room temperature of about 45 minutes. The consolidated pucks were machined into cubes (1×1×1 cm).

The cubes (n=3) were doped in pre-heated dicumyl peroxide (DCP) at 120° C. for 5 hours. The average weight gain of the virgin UHMWPE cubes was 76.7±2.0 mg. The excess DCP was wiped off the surface of the cubes before cooling down below the solidification temperature of DCP at around 40° C.

The cross-link density of the surface was measured using small sections close to the surface (approximately 0.5 mm×3×3 mm, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density of virgin, DCP-diffused and heated UHMWPE was 194±49 mol/m³. Thus, we demonstrated that cross-linking of UHMWPE without additives and blended with the antioxidant vitamin E could be achieved by diffusing DCP close to its T₁₀ (approximately 117° C. in this case) into UHMWPE for enough time to allow decomposition of the peroxide during diffusion.

One set of cubes without vitamin E, which were doped at 120° C. for 5 hours, was annealed further at 130° C. for 4 hours without doping. The cross-link density of these cubes was 342±21 mol/m³. This result demonstrated that peroxide cross-linking of UHMWPE could be achieved by diffusion close to the T₁₀ of the diffused peroxide and could be enhanced by subsequent annealing of the sample close to or above T₁₀.

The peroxide-diffused UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated cube.

### Example 11

### Cross-Linking of UHMWPE by Diffusion of Peroxides into Antioxidant-Containing UHMWPEs Followed by Heating (2) (Vitamin E as Antioxidant)

Vitamin E was blended with UHMWPE powder with the aid of isopropyl alcohol (IPA) as described in Example 1. The solvent was substantially removed from the polymer blend by evaporation at ambient pressure or vacuum. The concentration of vitamin E in the polymeric material was 0.1 wt % or 1.0 wt %. The virgin or blended UHMWPE powders were placed in a mold and were pre-heated at 180-190° C. in inert gas for about 1 hour. Then they were consolidated into pucks (diameter 10 cm, thickness 1 cm) with the press platens at 181° C. under a pressure of 20 MPa for 5 minutes with a cool-down to room temperature of about 45 minutes. The consolidated pucks were machined into cubes (1×1×1 cm).

The cubes (n=3) were doped in pre-heated Luperox® 130 (Table 2) at 100° C. for 4 hours. The excess peroxide was wiped off the surface of the cubes. The average weight gained by the 0.1 wt % and 1.0 wt % vitamin E-blended cubes was 7.4±0.2 and 6.6±0.0 mg, respectively. The cubes were then placed in a oven in argon gas at 180° C. and maintained at that temperature for 2 hours.

The cross-link density of the surface was measured using small sections close to the surface (0.5 mm×3×3 mm, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density of 0.1 wt % vitamin E-blended, Luperox® 130-diffused and heated UHMWPE was 227±19 mol/m³. The cross-link density of 1 wt % vitamin E-blended, Luperox® 130-diffused and heated UHMWPE was 178±18 mol/m³. Thus, we demonstrated that cross-linking of UHMWPE without additives and blended with the antioxidant vitamin E could be achieved by diffusing Luperox® 130 below its T₁₀ (approximately 131° C. in this case) into UHMWPE and heating the diffused polymer above its T₁₀ for enough time to allow decomposition of the peroxide.

The peroxide-diffused UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated cube.

### Example 12

### Cross-Linking by Irradiation and Peroxides (Vitamin E as Antioxidant)

Vitamin E is blended with polymeric material with the aid of a solvent such as isopropyl alcohol (IPA) as described in Example 1. The solvent is substantially removed from the vitamin E/polymeric material blend by evaporation at ambient pressure or vacuum. The polymeric material is optionally UHMWPE. The concentration of vitamin E in the polymeric material is 0 wt %, 0.001 wt %, 0.01 wt %, 0.1 wt %, 0.2 wt %, 0.5 wt %, 0.7 wt %, 1 wt %, 10 wt % or more. The virgin or blended UHMWPE powders are placed in a mold and are pre-heated at 180° C.-190° C. in inert gas, such as argon gas, for about 1 hour. Then they are consolidated into pucks (diameter 10 cm, thickness 1 cm) with the press platens at 181° C. and under a pressure of about 20 MPa for about 5 minutes with a cool-down to room temperature of about 45 minutes.

One group of the consolidated UHMWPE pucks are irradiated with gamma or electron beam irradiation at 25 kGy, 50 kGy, 75 kGy, 100 kGy, 125 kGy and 150 kGy in either in air or in inert gas or in vacuum packaging. Irradiated cubes (1×1×1 cm) are machined and the cubes are immersed in DCP at 60° C. for 4, 8, or 12 hours. The excess peroxide is wiped off the surface of the cubes above the solidification temperature of DCP. Then, the DCP-diffused, irradiated cubes are heated to 120° C., 130° C., or 140° C. in argon for 2, 4, 6, 8, or 10 hours. Another set of irradiated cubes are immersed in a BP emulsion (for example, obtained by making an emulsion of BP in water using an emulsifying agent such as Tween 20 or Span 80) at 40° C. for 4, 8, 12, 36 or 100 hours. The excess peroxide is wiped off the surface of the cubes above the solidification temperature of BP. Then, the BP-diffused, irradiated cubes are heated to 70° C., 100° C., or 120° C. in argon for 2, 4, 6, 8, or 10 hours. Another set of irradiated cubes are immersed in Luperox® 130 at 100° C. for 4, 8, or 12 hours. The excess peroxide is wiped off the surface of the cubes. Then, the L-130-diffused, irradiated cubes are heated to 150° C., 160° C., or 180° C. in argon for 2, 4, 6, 8, or 10 hours. Another set of irradiated cubes are immersed in Trigonox® 311 at 100° C. for 4, 8, or 12 hours. The excess peroxide is wiped off the surface of the cubes. Then, the T 311-diffused, irradiated cubes are heated to 180° C., 190° C., or 220° C. in argon for 2, 4, 6, 8, or 10 hours.

And other group of the consolidated UHMWPE pucks are machined into cubes (1×1×1 cm) and the cubes are immersed in DCP at 60° C. for 4, 8, or 12 hours. The excess peroxide is wiped off the surface of the cubes above the solidification temperature of DCP. Then, the DCP-diffused, cubes are heated to 120° C., 130° C., or 140° C. in argon for 2, 4, 6, 8, or 10 hours. Another set of cubes are immersed in a BP emulsion (for example, obtained by making an emulsion of BP in water using an emulsifying agent such as Tween 20 or Span 80) at 40° C. for 4, 8, 12, 36 or 100 hours. The excess peroxide is wiped off the surface of the cubes above the solidification temperature of BP. Then, the BP-diffused, cubes are heated to 70° C., 100° C., or 120° C. in argon for 2, 4, 6, 8, or 10 hours. Another set of cubes are immersed in Luperox® 130 at 100° C. for 4, 8, or 12 hours. The excess peroxide is wiped off the surface of the cubes. Then, the L-130-diffused, cubes are heated to 150° C., 160° C., or 180° C. in argon for 2, 4, 6, 8, or 10 hours. Another set of cubes are immersed in Trigonox® 311 at 100° C. for 4, 8, or 12 hours. The excess peroxide is wiped off the surface of the cubes. Then, the T 311-diffused, cubes are heated to 180° C., 190° C., or 220° C. in argon for 2, 4, 6, 8, or 10 hours. Finally the peroxide-diffused cubes are irradiated with gamma or electron beam irradiation at 25 kGy, 50 kGy, 75 kGy, 100 kGy, 125 kGy and 150 kGy in either in air or in inert gas or in vacuum packaging.

In this example the cubes shape is optionally replaced by an implant shape or an implant preform shape.

### Example 13

### Cross-Linking of UHMWPE by Diffusion of Peroxides into Antioxidant-Containing UHMWPE (Irganox® 1010 as Antioxidant)

Irganox® 1010 (Pentaerythritol Tetrakis(3-(3,5-di-tert-butyl-4-hydroxyphenyl)propionate) is blended with polymeric material with the aid of isopropyl alcohol (IPA) similar to the process described in Example 1 for vitamin E. The solvent is substantially removed from the polymer blend by evaporation at ambient pressure or vacuum. The concentration of Irganox® 1010 in the polymeric material is 0 wt %, 0.1 wt %, 0.2 wt %, 0.3 wt %, 0.5 wt %, 0.75 wt %, 1 wt %, 2 wt %, 3 wt %, or 5 wt %. The polymeric material is optionally UHMWPE. The virgin or blended UHMWPE powders are placed in a mold and were pre-heated at 180° C.-190° C. in inert gas for about 1 hour. Then they are consolidated into pucks (diameter 10 cm, thickness 1 cm) with the press platens at 190° C. and under a pressure of 20 MPa for 10 minutes with a cool-down to room temperature of about 3 hours. The consolidated pucks were then machined into cubes (1×1×1 cm).

Cubes are doped in pre-heated dicumyl peroxide (DCP) at 60° C. for 2, 4, 8, 16, or 32 hours or at 120° C. for 2, 4, 8 or 12 hours. The excess DCP is wiped off the surface of the cubes before cooling down below the solidification temperature of DCP at around 40° C. Some cubes doped with DCP are further heated to 115° C., 120° C., 125° C., 130° C., 135° C., 140° C., 145° C., 150° C., 155° C., 160° C., 170° C., 180° C., 190° C., 200° C., 250° C., or 300° C. in inert gas.

Another set of cubes (1×1×1 cm) are doped in pre-heated Luperox® 130 (L-130) at 100° C. for 2, 4, 8, 16, or 32 hours or at 150° C. for 2, 4, 8 or 12 hours. The excess L-130 is wiped off the surface of the cubes. Some cubes doped with L-130 are further heated to 150° C., 155° C., 160° C., 170° C., 180° C., 190° C., 200° C., 250° C., or 300° C. in inert gas.

Another set of cubes (1×1×1 cm) are doped in pre-heated Trigonox® 311 (T311) at 120° C. for 2, 4, 8, 16, or 32 hours or at 170° C. for 2, 4, 8 or 12 hours. The excess T311 is wiped off the surface of the cubes. Some cubes doped with T311 are further heated to 170° C., 180° C., 190° C., 200° C., 250° C., or 300° C. in inert gas.

The peroxide-doped cubes are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated cube.

### Example 14

### Cross-Linking of UHMWPE by Diffusion of Peroxides into Antioxidant-Containing UHMWPEs Followed by Heating (3) (Vitamin E as Antioxidant)

Vitamin E is blended with polymeric material with the aid of isopropyl alcohol (IPA) as described in Example 1. The solvent is substantially removed from the polymer blend by evaporation at ambient pressure or vacuum. The polymeric material is optionally UHMWPE. The concentration of vitamin E in the polymeric material is 0 wt %, 0.1 wt %, 0.2 wt %, 0.3 wt %, 0.5 wt %, 0.75 wt %, 1 wt %, 2 wt % or 5 wt %. The virgin or blended UHMWPE powders are placed in a mold and are pre-heated at 180° C.-190° C. in inert gas for about 1 hour. Then they are consolidated into pucks (diameter 10 cm, thickness 1 cm) with the press platens at 190° C. and under a pressure of about 20 MPa for 10 minutes with a cool-down to room temperature of about 3 hours. The consolidated pucks are machined into cubes (1×1×1 cm).

A paste is formed by blending hydrated benzoyl peroxide (BP) and an emulsifier such as Tween 20 at 50/50 wt %. Cubes are doped in this pre-heated BP (paste) at 50° C. for 4, 6, or 8 hours or longer. The excess BP is wiped off the surface of the cubes. The cubes are then heated in inert gas or air at 100° C. and maintained at that temperature for 2, 4, 6, 8 or 10 hours.

An emulsified solution of BP mixed with an emulsifier is made by adding water at elevated temperature and stirring. Another set of cubes (1×1×1 cm) are doped in this pre-heated BP (emulsion) at 50° C. for 4, 6, or 8 hours or longer. The excess BP is wiped off the surface of the cubes. The cubes are then heated in inert gas or air at 100° C. and maintained at temperature for 2, 4, 6, 8 or 10 hours or longer.

The peroxide-diffused vitamin E/polymeric material blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-diffused vitamin E/polymeric material blend. The irradiation is either before or after the peroxide diffusion.

### Example 15

### Manufacture of an Implant Using Peroxide Cross-Linking

Vitamin E is blended with UHMWPE powder whereby vitamin E constitutes 0.5 wt % of the blend. The blend is subsequently compression molded for consolidation into a near net shape implant preform. The near net shape implant preform is soaked in a peroxide bath or emulsion at below the peroxide initiation temperature (approximately T₁₀) for a duration long enough to diffuse sufficient amounts of peroxide into the near net shape implant to, subsequently, achieve enough cross-linking to reduce wear. The peroxide concentration in the consolidated near-net shape implant preform is about 0.1 wt %, 0.5 wt %, 1 wt %, 1.5 wt % or 2 wt % or higher. The peroxide is either uniformly or non-uniformly distributed throughout the implant preform. In the latter case, the peroxide concentration is calculated based on the overall weight of the implant preform and the total weight of peroxide diffused.

The near net shape UHMWPE implant is soaked in the peroxide bath or emulsion at 20° C., 40° C., 60° C., 80° C., 100° C., 120° C., 140° C., 160° C., 180° C. or 200° C. for sufficient time to achieve about an average of 1 wt % peroxide concentration in the first 2 millimeters of the near-net shape implant preform.

Subsequent to soaking in peroxide bath or emulsion, the near net shape implant preform is blotted dry and optionally heated. The heating is performed at 70° C., 80° C., 90° C., 100° C., 110° C., 120° C., 130° C., 140° C., 150° C., 160° C., 170° C., 180° C., 190° C., 200° C., 210° C., 220° C., 230° C., 240° C., 250° C., 260° C., 270° C., 280° C., 290° C., 300° C., 310° C., or 320° C. for 30 minutes, 1 hour, 2 hours, 2 hours, 4 hours, 5 hours, 6 hours, 7 hours, 8 hours, 9 hours, or 10 hours in inert gas or in air.

Following heating the near net shape implant is machined into a final implant shape, packaged, and sterilized. Sterilization is carried out using ionizing gamma irradiation, electron beam irradiation, ethylene oxide sterilization or gas plasma sterilization.

The peroxide-diffused UHMWPE/vitamin E blends of this example are optionally irradiated with gamma or electron beam irradiation at 25 kGy, 50 kGy, 75 kGy, 100 kGy, 125 kGy and 150 kGy in either in air or in inert gas or in vacuum packaging at a temperature between room temperature and 50° C. above the melting point of the irradiated peroxide-diffused UHMWPE/vitamin E blend before or after peroxide diffusion.

### Example 16

### Cross-Linking of Virgin (No Additive) and Vitamin E-Blended UHMWPE by Using Peroxide Blending (P130 or Luperox® 130)

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. These blends were further mixed with the desired amount of the peroxide. The virgin UHMWPE/peroxide blends and the UHMWPE/antioxidant/peroxide blends were placed in a mold and they were consolidated into pucks (diameter 10 cm, thickness 1 cm) with the press platens at the desired temperature and pressure (20 MPa) for about 2 hours followed by a cool-down for about three hours to room temperature under pressure. In this example the peroxide was Luperox®-130.

The vitamin E concentrations used were 0 wt %, 0.1 wt %, 0.2 wt %, 0.3 wt %, 0.4 wt %, 0.5 wt %, 0.6 wt %, 0.8 wt % and 1 wt %. The peroxide concentrations used were 0.5 wt %, 1 wt % and 1.5 wt %. Molding was done at 190° C.

Controls were 150-kGy irradiated vitamin E-blended GUR®1050 UHMWPE with different vitamin E concentrations and contained no added peroxides. These pucks were prepared in the same manner described with the exception of the consolidation time being 5 minutes instead of 2 hours.

The cross-link density of the consolidated pucks was measured using small sections (3×3×3 mm cubes, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density results are shown in Table 3 below. The numbers in parentheses in Table 3 are standard deviations.

TABLE 3
The cross-link density (mol/m³) of virgin and vitamin E-blended
UHMWPE cross-linked by Luperox ®-130 (P130) by blending
into powder and decomposing the peroxide during compression
molding as a function of peroxide concentration and in comparison
to radiation cross-linked (150 kGy) UHMWPE.

| | 0.5 wt % | 1 wt % | 1.5 wt % | |
|---|---|---|---|---|
| | P130 | P130 | P130 | 150 kGy |
| Virgin | 279 (5) | 343 (5) | 422 (14) | 239 (4) |
| 0.1 wt % | 250 (4) | 301 (5) | 355 (10) | 217 (7) |
| Vitamin-E | | | | |
| 0.2 wt % | 238 (6) | 297 (7) | 336 (8) | 193 (6) |
| Vitamin-E | | | | |
| 0.3 wt % | 244 (5) | 300 (9) | 347 (9) | |
| Vitamin-E | | | | |
| 0.5 wt % | 224 (5) | 279 (3) | 325 (4) | 131 (9) |
| Vitamin-E | | | | |
| 0.6 wt % | 235 (12) | 276 (5) | 321 (9) | |
| Vitamin-E | | | | |
| 0.8 wt % | 220 (8) | 259 (5) | 310 (7) | |
| Vitamin-E | | | | |
| 1 wt % | 193 (2) | 242 (6) | 294 (6) | 106 (9) |
| Vitamin-E | | | | |

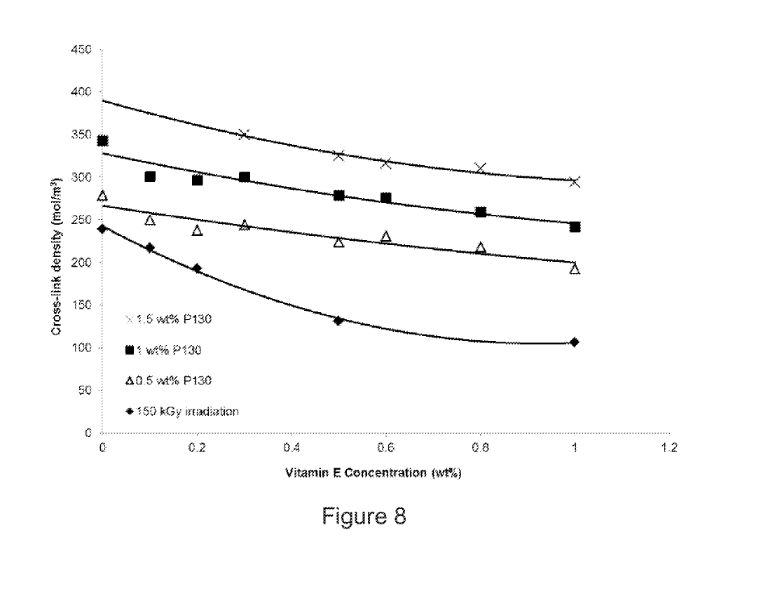

The results showed that (1) cross-linking was achieved during consolidation by using peroxide blending into vitamin E-blended UHMWPE; and (2) the free radical scavenging ability of vitamin E decreased cross-linking of UHMWPE compared to virgin (no additive) UHMWPE, but increasing vitamin E concentration was less effective against peroxide cross-linking than radiation cross-linking (FIG. 8).

The wear rate of peroxide cross-linked samples was measured by bidirectional pin-on-disc testing by rubbing 9 mm diameter and 13 mm height UHMWPE pains through a 5 by 10 millimeter rectangular crossing pattern at 2 Hz for 1.2 million cycles as described in Bragdon C et al., “A new pin-on-disc wear testing method for simulating wear of polyethylene on Cobalt-Chromium alloy in total hip arthroplasty”, J Arthroplasty 16:658-665 (2001). Wear was measured gravimetrically at 0.5 MC and at every 0.16 MC afterwards. The wear rate was calculated by the linear regression of the wear against number of cycles from 0.5 to 1.2 MC.

The wear rate results are shown in Table 4 below. The numbers in parentheses in Table 4 are standard deviations.

TABLE 4
The pin-on-disc wear rate (mg/million cycles) of virgin and vitamin
E-blended UHMWPE cross-linked by Luperox ®-130 (P130) during
compression molding as a function of peroxide concentration and
in comparison to radiation cross-linked (150 kGy) UHMWPE.

| | 0.5 wt % | 1 wt % | 1.5 wt % | 150 kGy |
|---|---|---|---|---|
| | P130 | P130 | P130 | P130 |
| Virgin | 1.51 (0.02) | 0.22 (0.02) | | 1.8 (1.1) |
| 0.1 wt % | 1.79 (0.13) | 0.32 (0.15) | | 2.0 (0.7) |
| Vitamin-E | | | | |
| 0.2 wt % | 2.76 (0.28) | 0.72 (0.03) | 0.26 (0.05) | 2.6 (0.9) |
| Vitamin-E | | | | |
| 0.3 wt % | 3.63 (0.23) | 0.69 (0.14) | 0.30 (0.05) | 3.5 (1.9) |
| Vitamin-E | | | | |
| 0.5 wt % | 4.76 (0.90) | 1.86 (0.21) | 0.53 (0.04) | 6.9 (0.6) |
| Vitamin-E | | | | |
| 0.6 wt % | 4.95 (0.62) | 1.94 (0.25) | 0.65 (0.09) | |
| Vitamin-E | | | | |
| 0.8 wt % | 5.46 (1.13) | 2.22 (0.04) | 0.81 (0.08) | |
| Vitamin-E | | | | |
| 1 wt % | 7.68 (1.03) | 2.41 (0.06) | 1.91 (0.21) | 5.7 (2.7) |
| Vitamin-E | | | | |

The results showed that low (1-2 mg/MC) and extremely low wear rates (<1 mg/MC) could be obtained using peroxide cross-linking of vitamin E-blended UHMWPE. Also, at each vitamin E concentration, all peroxide cross-linked UHMWPEs had lower wear rates than radiation cross-linked UHMWPE using 150 kGy of radiation dose.

Tensile testing was performed on Type V dogbones in accordance with ASTM D638. Thin sections (3.2 mm-thick) were machined from the peroxide cross-linked pucks, out of which dogbones were stamped. The dogbones were tested in tension at a crosshead speed of 10 mm/min (Insight 2, MTS, Eden Prairie, Minn., USA). The strain was measured by a laser extensometer.

The ultimate tensile strength (UTS) of peroxide cross-linked UHMWPEs are reported in Table 5 below. The numbers in parentheses in Table 5 are standard deviations.

TABLE 5
The ultimate tensile strength (MPa) of virgin and vitamin
E-blended UHMWPE cross-linked by Luperox ®-130 (P130)
by blending into powder and decomposing the peroxide during
compression molding as a function of peroxide concentration
and in comparison to radiation cross-linked (150 kGy) UHMWPE.

| | No cross- | 0.5 wt % | 1 wt % | 1.5 wt % | |
|---|---|---|---|---|---|
| | linking | P130 | P130 | P130 | 150 kGy* |
| Virgin | 45.4 (2.7) | | | 33.6 (1.4) | 46.0 (4.6) |
| 0.1 wt % | | | | 26.8 (2.2) | 40.4 (2.5) |
| Vitamin-E | | | | | |
| 0.2 wt % | 44.5 (3.4) | 37.1 (3.2) | 28.6 (2.2) | 27.9 (3.3) | |
| Vitamin-E | | | | | |
| 0.3 wt % | 49.5 (5.7) | 41.3 (3.8) | 31.3 (2.9) | 38.1 (3.8) | 52.6 (1.7) |
| Vitamin-E | | | | | |
| 0.5 wt % | 51.2 (8.3) | 48.0 (3.5) | 37.5 (2.3) | 32.3 (2.9) | |
| Vitamin-E | | | | | |
| 0.6 wt % | 51.1 (10.7) | 49.4 (4.5) | 37.6 (3.8) | 31.0 (3.7) | |
| Vitamin-E | | | | | |
| 0.8 wt % | 56.6 (3.9) | 48.2 (7.5) | 40.5 (3.1) | 33.0 (3.3) | |
| Vitamin-E | | | | | |
| 1 wt % | 51.0 (4.8) | 39.9 (4.6) | 38.3 (1.9) | 36.2 (2.2) | 53.0 (5.2) |
| Vitamin-E | | | | | |

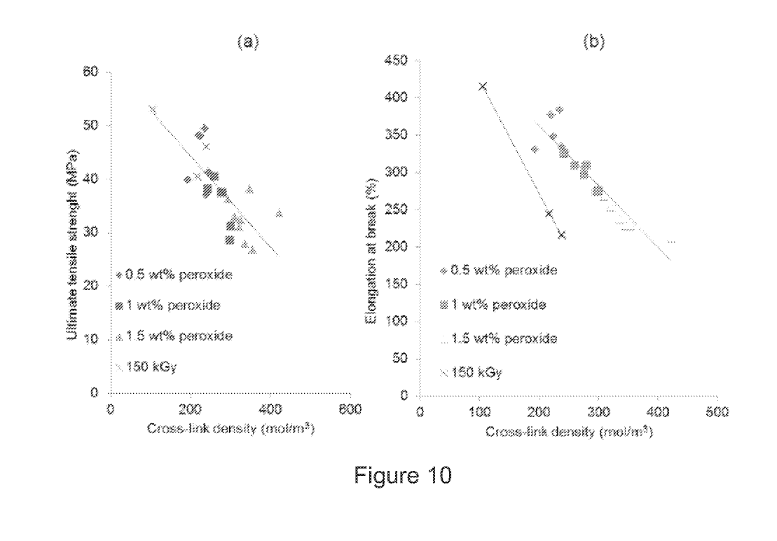

At the similar wear rate, for example for 0.5 wt % vitamin E-blended and 1 wt % peroxide cross-linked UHMWPE (1.86 mg/MC) and virgin, 150 kGy irradiated UHMWPE (1.8 mg/MC), the UTS was comparable (48 and 46 MPa, respectively). The UTS had a similar and strong correlation with cross-link density for both radiation and peroxide cross-linked UHMWPEs (FIG. 10a). In contrast, the elongation-at-break (EAB) of peroxide cross-linked UHMWPEs were higher than those of the radiation cross-linked blends at similar cross-link density (Table 6 below and FIG. 10b). The numbers in parentheses in Table 6 are standard deviations.

TABLE 6
The elongation at break (%) of virgin and vitamin E-blended
UHMWPE cross-linked by Luperox ®-130 (P130) by blending
into powder and decomposing the peroxide during compression
molding as a function of peroxide concentration and in comparison
to radiation cross-linked (150 kGy) UHMWPE.

| | No cross- | 0.5 wt % | 1 wt % | 1.5 wt % | |
|---|---|---|---|---|---|
| | linking | P130 | P130 | P130 | 150 kGy |
| Virgin | 372 (11) | | | 212 (4) | 216 |
| 0.1 wt % | | | | 228 (12) | 244 |
| vitamin E | | | | | |
| 0.2 wt % | 366 (17) | 334 (15) | 274 (11) | 237 (16) | |
| Vitamin-E | | | | | |
| 0.3 wt % | 373 (17) | 328 (18) | 276 (15) | 228 (17) | 297 |
| Vitamin-E | | | | | |
| 0.5 wt % | 390 (25) | 348 (13) | 310 (12) | 260 (9) | |
| Vitamin-E | | | | | |
| 0.6 wt % | 379 (37) | 384 (11) | 297 (21) | 254 (15) | |
| Vitamin-E | | | | | |
| 0.8 wt % | 432 (18) | 377 (33) | 309 (14) | 268 (19) | |
| Vitamin-E | | | | | |
| 1 wt % | 375 (32) | 331 (21) | 326 (10) | 287 (7) | 415 |
| Vitamin-E | | | | | |

Vitamin E concentration profiles were determined by using Fourier Transform Infrared Spectroscopy (FTIR). Thin (150 μm) cross sections were microtomed from the peroxide cross-linked pucks; these were then analyzed on an infrared microscope (BioRad UMA 500, Bio-Rad, Cambridge, Mass., USA). Vitamin E index was calculated as the ratio of the area under the α-tocopherol 1265 cm⁻¹ peak (1245 to 1275 cm⁻¹) to the area under the crystalline polyethylene 1895 cm⁻¹ peak (1850-1985 cm⁻¹) after subtracting the respective baselines. The vitamin E index was measured before and after subjecting the thin cross-sections to extraction by boiling hexane for 16 hours and drying in vacuum for 24 hours. The ratio of grafted vitamin E was calculated as the vitamin E index after hexane extraction to the vitamin E index measured on thin sections cut from uncross-linked vitamin E-blended UHMWPE of the same concentration.

The graft ratios are shown in Table 7 below. The numbers in parentheses in Table 7 are standard deviations.

TABLE 7
Ratio of grafted vitamin E in vitamin E-blended UHMWPE cross-
linked by Luperox ® 130 (P130) during compression
molding compared to radiation cross-linked (150 kGy) UHMWPE.

| | 0.5 wt % | 1 wt % | 1.5 wt % | |
|---|---|---|---|---|
| | P130 | P130 | P130 | 150 kGy |
| Virgin | NA | NA | NA | NA |
| 0.1 wt % | | | | |
| Vitamin-E | | | | |
| 0.2 wt % | | | | |
| Vitamin-E | | | | |
| 0.3 wt % | 38.4 (1.1) | 36.4 (2.4) | 23.9 (2.8) | 29 (4) |
| Vitamin-E | | | | |
| 0.5 wt % | | 42.2 (5.8) | 41.6 (6.1) | 40 (2) |
| Vitamin-E | | | | |
| 0.6 wt % | | 39.1 (1.1) | 50.3 (10.0) | ~28 (5) |
| Vitamin-E | | | | |
| 0.8 wt % | | 44.4 (3.6) | 47.4 (6.9) | |
| Vitamin-E | | | | |
| 1 wt % | 17.8 (1.7) | 42.5 (0.5) | 47.5 (1.5) | 16 (1) |
| Vitamin-E | | | | |

The amount of grafted vitamin E of peroxide cross-linked UHMWPEs, expected to be immobilized in cross-linked UHMWPE, was generally equivalent to or higher than radiation cross-linked UHMWPEs (see Table 7).

Cubes (10 mm) were machined from the cross-linked blends. Then, the cubes were doped with squalene at 110° C. for 1 hour and cooled down to room temperature. The average amount of squalene absorbed into cross-linked UHMWPEs was 21 mg. After squalene doping, the samples were placed in a pressure vessel at 70° C. at 5 atm. of oxygen for 14 days. Oxidation profiles were determined by using FTIR. The cubes were first cut in half and 150 μm thin cross sections were microtomed from the inner surfaces. These thin sections were extracted by boiling hexane for 16 hours and dried under vacuum for 24 hours. These were then analyzed on an infrared microscope (BioRad UMA 500, Bio-Rad, Cambridge, Mass., USA) as a function of depth away from the surface. An oxidation index was calculated as the ratio of the area under the carbonyl peaks at 1740 cm⁻¹ (1680 to 1780 cm⁻¹) to the area under the methylene vibration at 1370 cm⁻¹ (1330 to 1390 cm⁻¹) peak. Measurements were made on three separate thin sections. An average surface oxidation index was calculated as an average of the surface 1.5 mm of the samples.

The average surface oxidation index was below 0.025 for all tested samples after accelerated aging (0.5 wt % vitamin E/1 wt % peroxide; 0.6 wt % vitamin E/1 wt % peroxide; 0.5 wt % vitamin E/1.5 wt % peroxide; 0.6 wt % vitamin E/1.5 wt % peroxide; 0.8 wt % vitamin E/1.5 wt % peroxide; 0.8 wt % vitamin E/1.5 wt % peroxide).

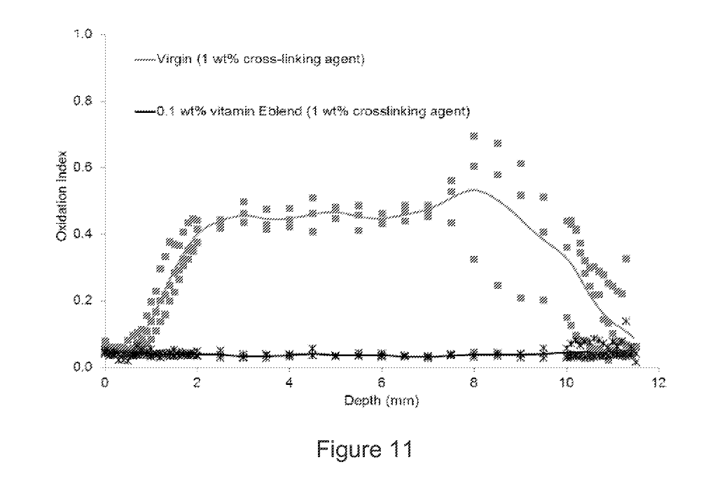

In a separate test, virgin/1 wt % peroxide UHMWPE blend was compared to 0.1 wt % vitamin E/1 wt % peroxide/UHMWPE blend in accelerated aging for 14 days at 5 atm. of oxygen at 70° C. without doping with squalene. The vitamin E-containing samples showed much less oxidation than the virgin, peroxide cross-linked UHMWPE (FIG. 11). Together, these results showed that antioxidant stabilization of peroxide cross-linked UHMWPE against oxidation was possible and the oxidation resistance was improved compared to virgin UHMWPE.

Crystallinity was measured using differential scanning calorimetry (DSC, Q1000, TA Instruments, Delaware, USA). The samples were heated at 10° C./min from −20 to 200° C. and the heat flow was recorded. The crystallinity was calculated by taking the area under this curve from 20 to 160° C. and normalizing the value to the enthalpy of fusion of 100% crystalline polyethylene; 291 J/g.

The crystallinity values for peroxide cross-linked blends are shown in Table 8 below. The numbers in parentheses in Table 8 are standard deviations.

TABLE 8
The crystallinity (%) of virgin and vitamin E-blended UHMWPE
cross-linked by Luperox ®-130 (P130) by blending
into powder and decomposing the peroxide during compression
molding as a function of peroxide concentration and in
comparison to radiation cross-linked (150 kGy) UHMWPE.

| | No cross- | 0.5 wt % | 1 wt % | 1.5 wt % | |
|---|---|---|---|---|---|
| | linking | P130 | P130 | P130 | 150 kGy |
| Virgin | 55.1 (0.8) | 48.5 (0.3) | 47.5 (0.2) | | 60.3 (0.3) |
| 0.1 wt % | 55.1 (0.2) | 48.0 (0.6) | 45.5 (0.4) | | 53.0 (0.2) |
| Vitamin-E | | | | | |
| 0.2 wt % | 53.7 (0.1) | 49.1 (0.5) | 45.5 (0.3) | | 57.9 (1.4) |
| Vitamin-E | | | | | |
| 0.3 wt % | | 48.2 (0.6) | 46.0 (0.8) | | |
| Vitamin-E | | | | | |
| 0.5 wt % | | 49.7 (1.0) | 44.9 (4.5) | 44.2 (0.4) | 57.1 (1.2) |
| Vitamin-E | | | | | |
| 0.6 wt % | | | 47.2 (0.4) | 44.5 (1.5) | |
| Vitamin-E | | | | | |
| 0.8 wt % | | | 47.1 (1.4) | | |
| Vitamin-E | | | | | |
| 1 wt % | 53.0 (0.2) | 50.6 (0.6) | 49.4 (0.2) | 46.7 (0.7) | 55.4 (1.8) |
| Vitamin-E | | | | | |

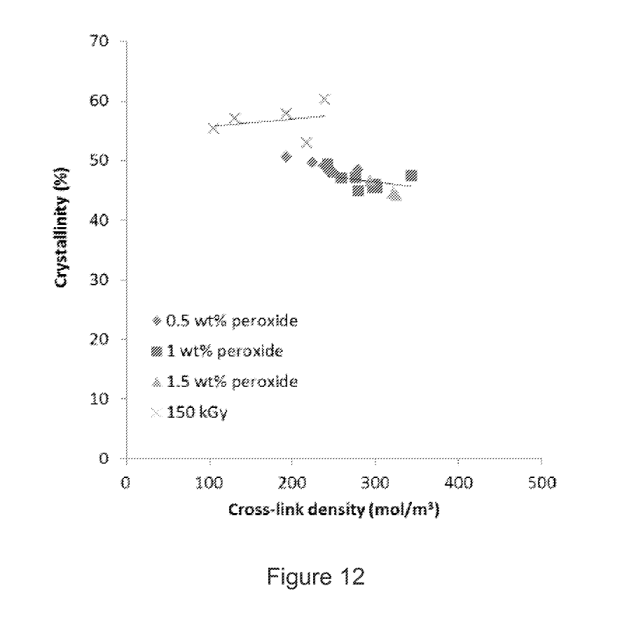

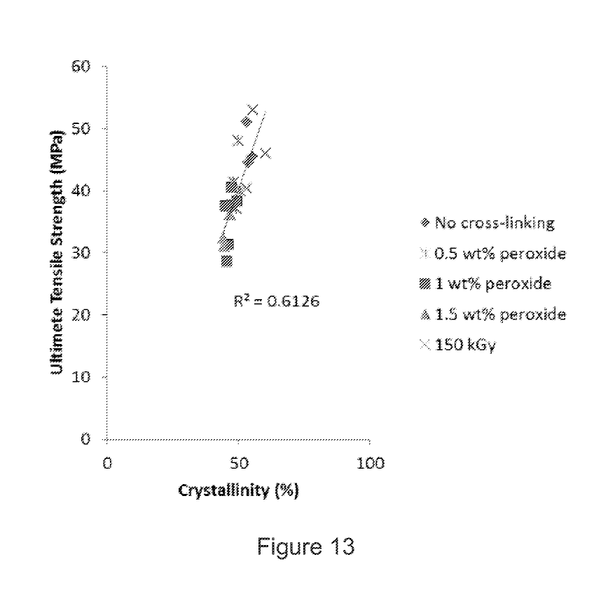

The crystallinity values for peroxide cross-linked UHMWPEs were generally lower than radiation cross-linked blends because the cross-linking of peroxide blends took place at above the melting point of the UHMWPE. In this way, the polymer was crystallized in the presence of cross-links, which is known to reduce the crystallinity (FIG. 12). The changes in crystallinity affected the ultimate tensile strength (FIG. 13) but the elongation at break was higher for peroxide cross-linked UHMWPEs (FIG. 10b) in comparison to the radiation cross-linked UHMWPE controls, thus maintaining toughness.

### Example 17

### The Effect of Molding Conditions on Peroxide Cross-Linked UHMWPE (P130 or Luperox® 130)

A series of manufacturing conditions were used to prepare peroxide cross-linked UHMWPE using 0.1 wt % vitamin E-blended GUR®1050 UHMWPE blended with 0.5 wt % Luperox® 130 as the cross-linking agent. These conditions are outlined in Table 9 below.

TABLE 9
The processing parameters for 0.1 wt % vitamin E-blended UHMWPE
cross-linked by blending with Luperox ® 130 and decomposing
the peroxide during manufacturing by consolidation

| SAMPLE | PROCESSING PARAMETERS |
|---|---|
| P130-1 | Heating in Oven: 135° C. 1.5 hours |
| | Preheating in Press: 25 min |
| | Dwell Pressure: 20 MPa |
| | Dwell Temperature: 180° C. |
| | Dwell Time: 5 min |
| P130-2 | Heating in Oven: 135° C. 1.5 hours |
| | Preheating in Press: 25 min |
| | Dwell Pressure: 15 MPa |
| | Dwell Temperature: 180° C. |
| | Dwell Time: 15 min |
| P130-3 | Heating in Oven: 135° C. 1.5 hours |
| | Preheating in Press: 25 min |
| | Dwell Pressure: 20 MPa |
| | Dwell Temperature: 170° C. |
| | Dwell Time: 15 min |
| P130-4 | Heating in Oven: 135° C. 1.5 hours |
| | Preheating in Press: Nil |
| | Dwell Pressure: 20 MPa |
| | Dwell Temperature: 180° C. |
| | Dwell Time: 40 min |
| P130-5 | Heating in Oven: 135° C. 1.5 hours |
| | Preheating in Press: 25 min |
| | Dwell Pressure: 20 MPa |
| | Dwell Temperature: 200° C. |
| | Dwell Time: 15 min |
| P130-6 | Heating in Oven: Nil |
| | Preheating in Press: Nil |
| | Dwell Pressure: 20 MPa |
| | Dwell Temperature: 180° C. |
| | Dwell Time: 2 hours |

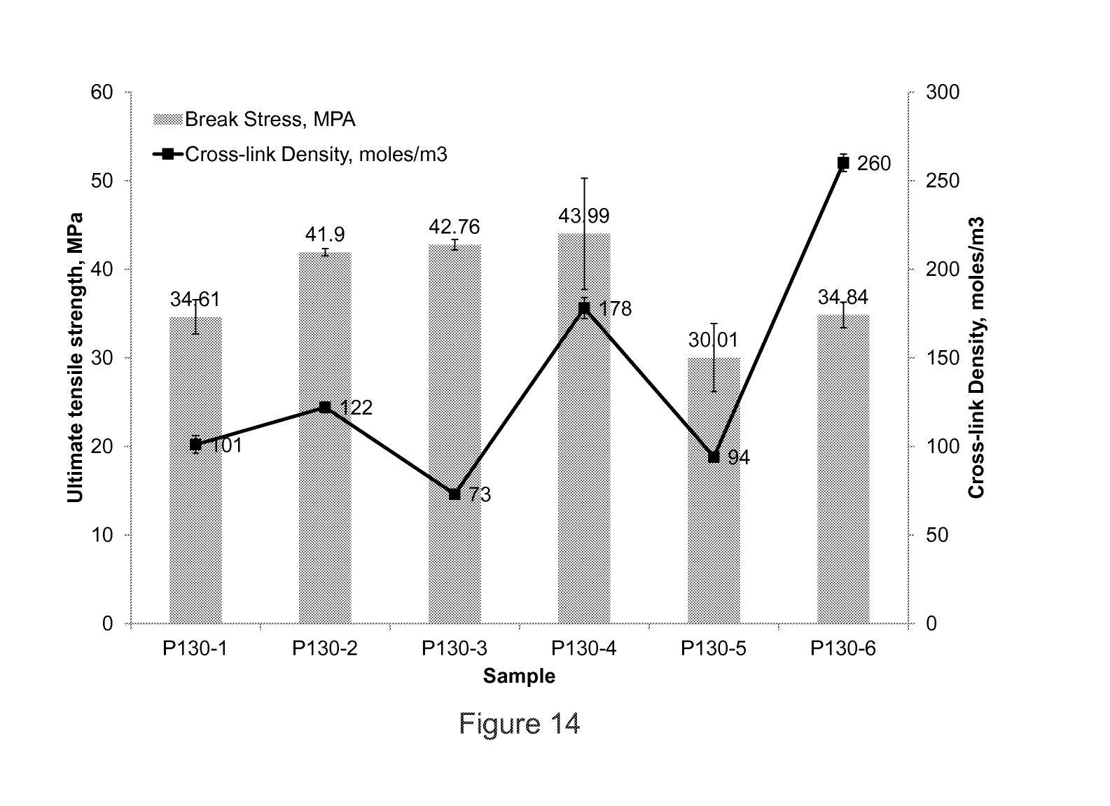

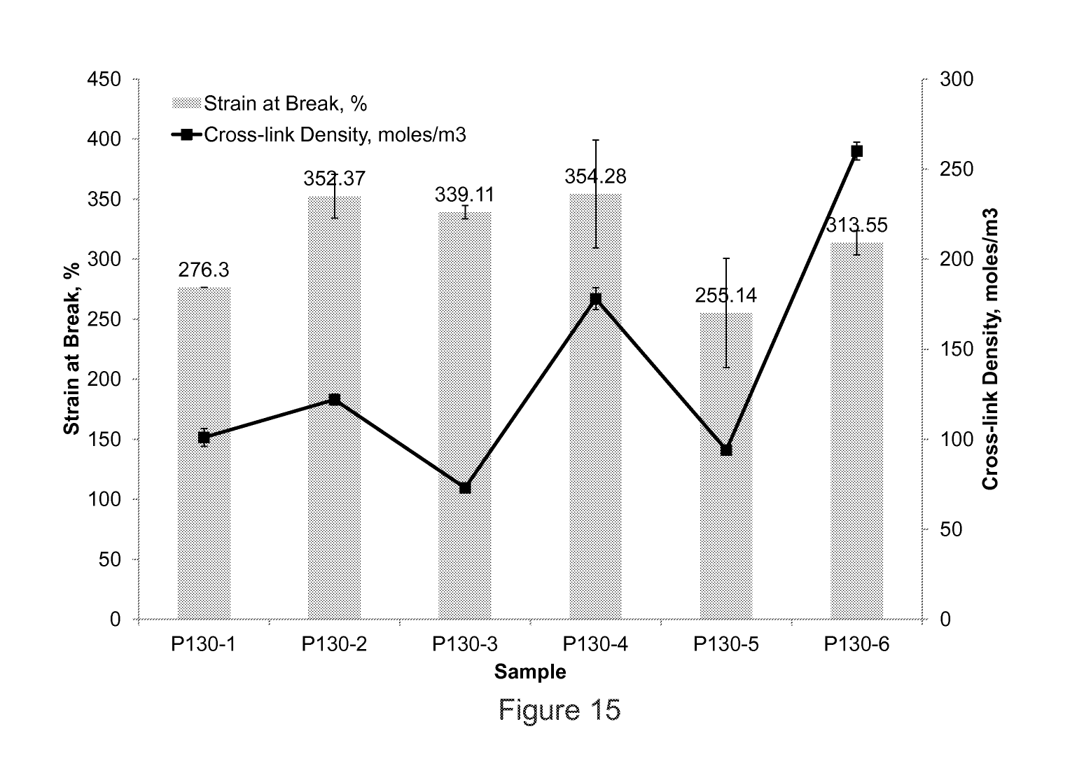

The cross-link density, the ultimate tensile strength and the strain-at-break of the samples are shown in FIGS. 14 and 15. The results suggested that there is wide variability in the properties of the material depending on processing conditions.

### Example 18

### Cross-Linking of Virgin and Vitamin E-Blended UHMWPE Using Peroxide Cross-Linking (Trigonox® 311 or T311)

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. These blends were further mixed with the desired amount of the peroxide. The virgin UHMWPE/peroxide blends and the UHMWPE/antioxidant/peroxide blends were placed in a mold and they were consolidated into pucks (diameter 10 cm, thickness 1 cm) with the press platens at the desired temperature and pressure (20 MPa) for about 2 hours followed by a cool-down for about three hours to room temperature under pressure. In this example the peroxide was Trigonox® 311 (T311).

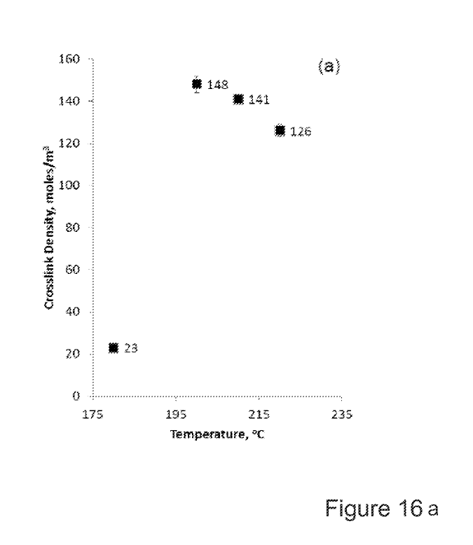

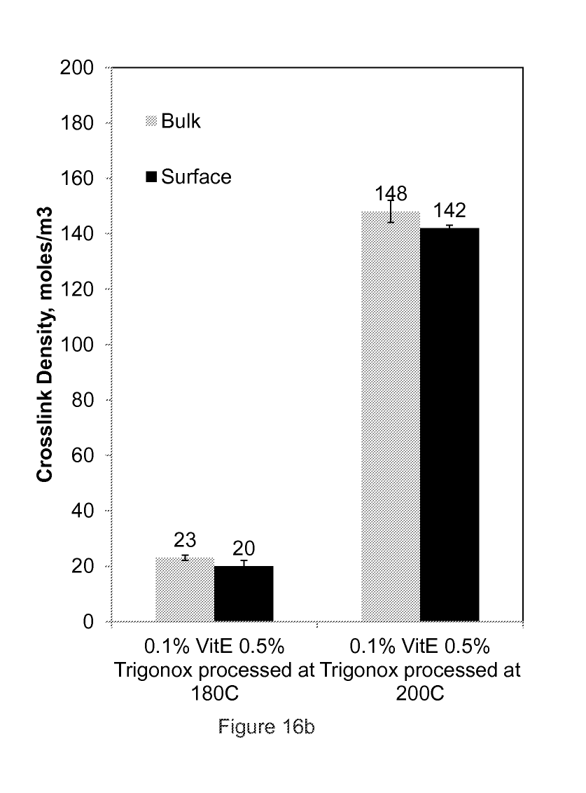

A 0.1 wt % vitamin E-blended UHMWPE was prepared with 0.5 wt % Trigonox® 311 at 180° C., 200° C., 210° C. or 220° C. The cross-link density of the surface and bulk of the pucks were similar suggesting that there was no gradient in temperature during processing (FIG. 16). Also, the cross-link density at 180° C. was much lower than that at 200° C., indicating that there was less decomposition of the peroxide at the lower temperature causing less cross-linking. The T₁ of T311 was 184° C., therefore at 180° C., not enough of the peroxide was decomposed to cause substantial cross-linking.

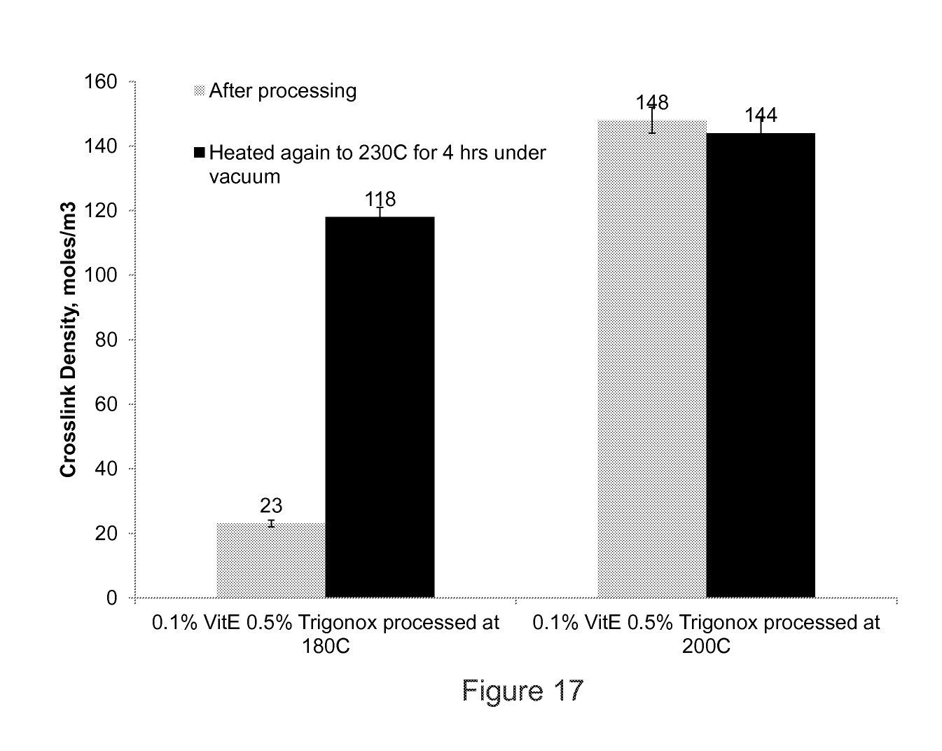

When these pucks were heated to above T₁ at 230° C. for 4 hours under vacuum after consolidation, the cross-link density of the puck molded at 180° C. increased significantly whereas that of the puck molded at 200° C. did not change (FIG. 17). This result also suggested that molding below T₁ (or the defined decomposition temperature of the used peroxide) would limit decomposition during consolidation but cross-linking could be increased further by heating the consolidated material above the decomposition temperature of the peroxide.

Virgin and 0.1 wt % vitamin E-blended UHMWPE were cross-linked using 1 wt % Trigonox® 311. The cross-link density was 136±3 and 149±2 mol/m³, respectively.

The wear rate of peroxide cross-linked samples was measured by bidirectional pin-on-disc testing, described above, with a 5 by 10 mm rectangular crossing pattern at 2 Hz for 1.2 million cycles. Wear was measured gravimetrically at 0.5 MC and at every 0.16 MC afterwards. The wear rate was calculated by the linear regression of the wear against number of cycles from 0.5 to 1.2 MC. The wear rates of virgin and 0.1 wt % vitamin E-blended UHMWPE cross-linked using 1 wt % Trigonox® 311 were 2.74±1.04 and 1.85±0.44 mg/MC, respectively. These results suggested that despite the fact that the cross-link density of UHMWPEs cross-linked using T311 were lower than those crosslinked using P130, low wear rates could still be obtained. The wear rate of uncrosslinked 0.1 wt % vitamin E-blended UHMWPE was 12.02±0.36 mg/MC.

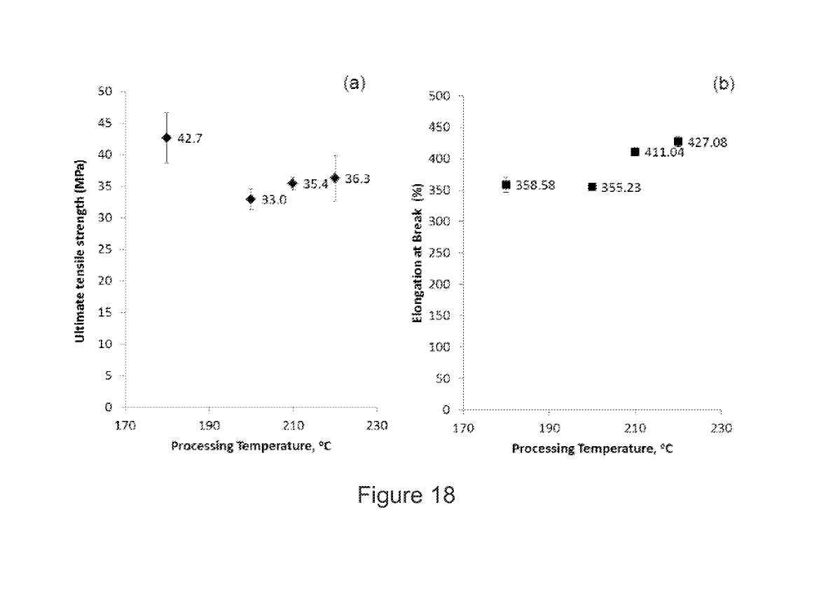

The ultimate tensile strength and the elongation-at-break of 0.1 wt % vitamin E-blended UHMWPEs cross-linked using T311 by consolidation at different temperatures are shown in FIG. 18.

Cubes (10 mm) were machined from virgin and 0.1 wt % vitamin E-blended UHMWPE cross-linked using 1 wt % Trigonox® 311. The cubes were placed in a pressure vessel at 70° C. at 5 atm. of oxygen for 14 days. Oxidation profiles were determined by using FTIR. The cubes were cut in half and 150 μm cross sections were microtomed from the inner surfaces. These thin sections were extracted by boiling hexane for 16 hours and drying under vacuum for 24 hours. These were then analyzed on an infrared microscope (BioRad UMA 500, Bio-Rad, Cambridge, Mass., USA) as a function of depth away from the surface. An oxidation index was calculated, as described above, as the ratio of the area under the carbonyl peaks at 1700 cm⁻¹ to the area under the methylene vibration at 1370 cm⁻¹ peak. Measurements were made on three separate thin sections.

After accelerated aging, there was oxidation in the virgin, cross-linked UHMWPE whereas the oxidation level of the 0.1 wt % vitamin E blend was 0.08. This result suggested that the oxidative stability of UHMWPE cross-linked using Trigonox® 311 was improved by the addition of vitamin E in the blend.

### Example 19

### Cross-Linking of Antioxidant Blends of UHMWPE Using Diffusion of Peroxides (DCP)

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. The virgin UHMWPE and the UHMWPE/antioxidant blends were placed in a mold and they were consolidated into pucks (diameter 10 cm, thickness 1 cm) with the press platens at the desired temperature and pressure (20 MPa) for about 10 minutes followed by a cool-down for about three hours to room temperature under pressure.

Cubes (10 mm) were machined from 0.1 wt % vitamin E-blended UHMWPE pucks. Three cubes each were doped in dicumyl peroxide (DCP) in a glass flask in an oil bath at 60° C., 80° C. or 100° C. for 4 hours under argon flow. The doped cubes were cooled, then annealed at 130° C. under argon flow for 4 hours for the decomposition of the peroxides. Similarly, three cubes each were doped with Luperox®-130 (P130) at 80° C., 100° C. or 120° C. for 4 hours, then annealed at 180° C. for 4 hours.

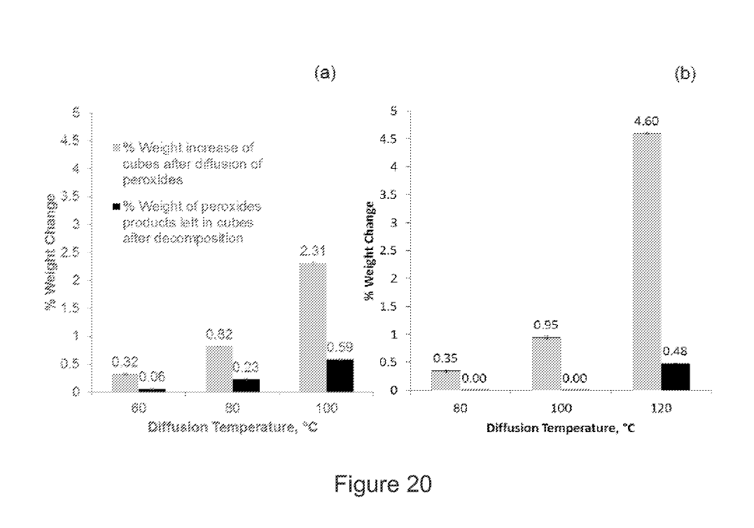

The weight gained by the cubes increased with increasing temperature for both DCP (FIG. 20a) and P130 (FIG. 20b). After the decomposition of the peroxides and cross-linking, the cubes lost weight. In some cases, such as that of DCP-doped samples at 60° C. and P130-doped samples at 80° C. and 100° C., all of the weight gained during doping was lost during the subsequent annealing step. This is because of the evaporation of all peroxides and peroxide decomposition products from the peroxide diffused and cross-linked UHMWPEs.

The cross-link density of the consolidated pucks was measured using small sections (approximately 3×3 mm sections from the first 1 mm for ‘surface’ and 3 mm cubes from the center for ‘bulk’, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

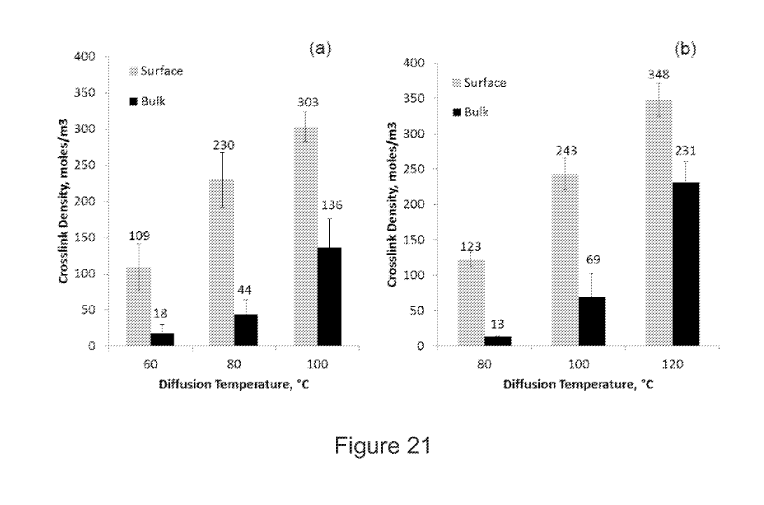

The cross-link density of peroxide diffused and annealed UHMWPEs increased with increasing doping temperature for both DCP-doped (FIG. 21a) and P130-doped (FIG. 21b) UHMWPEs. The cross-link density of the surface was higher than the bulk in each case.

The wear rate of peroxide cross-linked samples was measured by bidirectional pin-on-disc testing, as described above, with a 5 by 10 mm rectangular crossing pattern at 2 Hz for 1.2 million cycles (MC). Wear was measured gravimetrically at 0.5 MC and at every 0.16 MC afterwards. The wear rate was calculated by the linear regression of the wear against number of cycles from 0.5 to 1.2 MC.

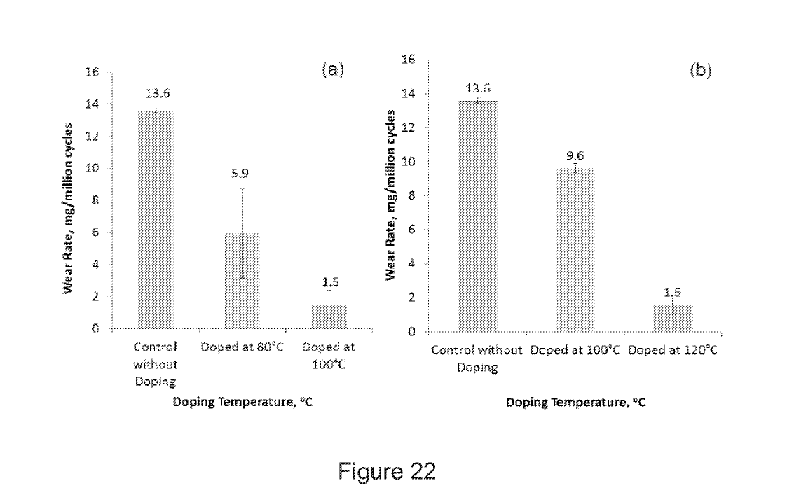

The wear rates of 0.1 wt % vitamin E-blended UHMWPE doped with DCP at 80° C. or 100° C. then annealed at 130° C. were much lower than that of uncross-linked UHMWPE (FIG. 22a). The wear rate of 0.1 wt % vitamin E-blended UHMWPE doped with DCP at 100° C. and annealed at 130° C. was in the wear rate range of 1-2 mg/MC, which has been a desirable range because previous radiation cross-linked UHMWPEs (100 kGy irradiated and melted) in clinical use, which have shown low wear rates in vivo (see Leung et al. “Incidence of pelvic osteolysis at early follow-up with highly cross-linked and noncross-linked polyethylene”, J Arthroplasty 22: 134-139 (2007)) showed this wear rate in vitro under similar conditions (see Muratoglu et al., “Unified wear model for highly cross-linked UHMWPE”, Biomaterials 20(16): 1463-1470 (1999)).

Similarly, the wear rates of 0.1 wt % vitamin E-blended UHMWPE doped with P130 at 100° C. or 120° C. then annealed at 180° C. were much lower than that of uncross-linked UHMWPE (FIG. 22b). The wear rate of 0.1 wt % vitamin E-blended UHMWPE doped with P130 at 120° C. and annealed at 180° C. was in the wear rate range of 1-2 mg/MC.

These results suggested that this novel technique of diffusing a peroxide into consolidated UHMWPE at a temperature below its T₁ (137 and 152° C., respectively for DCP and P130), then annealing at a temperature close to or above its T₁ could result in cross-linking. In this manner, it was also possible to cause cross-linking in UHMWPE below the melting point of the polymer by choosing a peroxide, whose decomposition temperature was close to or below the melting point of the polymer, DCP in this case.

### Example 20

### The Effect of Decomposition Temperature on Peroxide-Diffused UHMWPE Properties

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. The virgin UHMWPE and the UHMWPE/antioxidant blends were placed in a mold and they were consolidated into pucks (diameter 10 cm, thickness 1 cm) with the press platens at the desired temperature and pressure (20 MPa) for about 10 minutes followed by a cool-down for about three hours to room temperature under pressure.

Cubes (10 mm) were machined from 0.1 wt % vitamin E-blended UHMWPE pucks. Cubes were doped in dicumyl peroxide (DCP) in a glass flask in an oil bath at 80° C. for 4 hours under argon flow. The doped cubes were cooled, then three cubes each were annealed at either 130° C. or 140° C. under argon flow for 4 hours for the decomposition of the peroxide. Similarly, cubes were doped with Luperox®-130 (P130) at 100° C. for 4 hours, then annealed at 150° C., 165° C. or 180° C. for 4 hours.

The cross-link density of the consolidated pucks was measured using small sections (approximately 3×3 mm sections from the first 1 mm for ‘surface’ and 3 mm cubes from the center for ‘bulk’, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

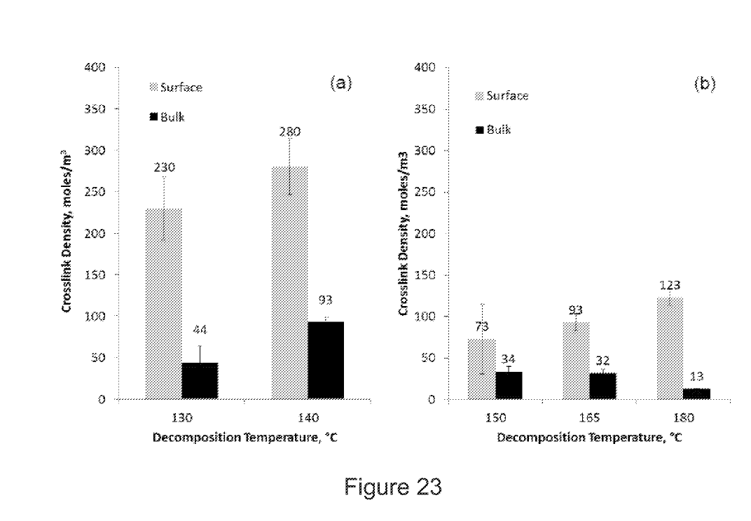

The cross-link density of peroxide diffused and annealed UHMWPEs increased slightly with increasing decomposition temperature for both DCP-doped (FIG. 23a) and P130-doped (FIG. 23b) UHMWPEs. The cross-link density of the surface was higher than the bulk in each case.

There is a competition between the diffusion of the peroxide into the polymer and the decomposition of the peroxide that leads to cross-linking at temperatures close to or above the decomposition temperature of the peroxide. Therefore, there may be an optimum temperature of decomposition for each peroxide where the decomposition is fast enough that there is no substantial diffusion into the polymer that results in bulk cross-linking. Thus, changing the doping and annealing temperatures can be used not only to change the peroxide content and therefore the crosslinking level but also for spatial control of cross-linking throughout the thickness of the desired sample.

### Example 21

### The Effect of Decomposition Time and Repeated Heating on Cross-Link Density

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. The virgin UHMWPE and the UHMWPE/antioxidant blends were placed in a mold and they were consolidated into pucks (diameter 10 cm, thickness 1 cm) with the press platens at the desired temperature and pressure (20 MPa) for about 10 minutes followed by a cool-down for about three hours to room temperature under pressure. Cubes (10 mm) were machined from 0.1 wt % vitamin E-blended UHMWPE pucks. Cubes (n=3 each) were doped in Luperox® 130 (P130) in a glass flask in an oil bath at 80° C. for 4 hours under argon flow followed by annealing at 150° C. under argon flow for 4 or 10 hours for the decomposition of the peroxide. Another set of cubes (n=3 each) were doped in Luperox® 130 (P130) in a glass flask in an oil bath at 100° C. for 4 hours under argon flow followed by annealing at 180° C. under argon flow for 2 or 4 hours for the decomposition of the peroxide. Another set of cubes (n=3 each) were doped in Luperox® 130 (P130) in a glass flask in an oil bath at 80° C. for 4 hours under argon flow followed by annealing at 150° C. under argon flow for 10 hours followed by further annealing at 180° C. under argon flow for 10 hours.

The cross-link density of the consolidated pucks was measured using small sections (approximately 3 mm cubes from the center, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density of the samples annealed at 150° C. for 4 hours was 73±42 mol/m³, that of samples annealed at 180° C. for 2 hours was 227±19 mol/m³, and that of those annealed first at 150° C., then at 180° C. was 52±14 mol/m³. These results suggested that after doping with the peroxide, there was an optimum temperature range where peroxide decomposition and cross-linking can occur. If the temperature is below this range, then the peroxide can decompose but not cause the desired cross-linking level. It is possible that at lower temperatures of annealing there is some evaporation of peroxide; therefore leaving fewer peroxide molecules to decompose and cross-link the polymeric material during the subsequent high temperature annealing step.

The cross-link density of the samples annealed at 150° C. for 10 hours was 63±31 mol/m³ and those annealed at 180° C. for 4 hours was 243±22 mol/m³. This result suggested that once the desired temperature of decomposition is reached, decomposition and cross-linking of the polymer was relatively fast with no further improvement at this decomposition temperature.

### Example 22

### Spatial Control of Cross-Linking by Layered Molding of Peroxide Blended UHMWPE and UHMWPE without Peroxides

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. The virgin UHMWPE and the UHMWPE/antioxidant blends were placed in a mold and they were consolidated into pucks (diameter 10 cm, thickness 1 cm) with the press platens at the desired temperature and pressure (20 MPa) for about 2 hours followed by a cool-down for about three hours to room temperature under pressure. Approximately 20 g of 0.1 wt % vitamin E and 0.5 wt % Luperox® 130 (P130) blended GUR® 1050 UHMWPE was layered molded with 80 g of 0.1 wt % vitamin E-blended GUR® 1050 UHMWPE. Similarly, approximately 20 g of 0.1 wt % vitamin E and 1 wt % Luperox® 130 (P130) blended GUR® 1050 UHMWPE was layered molded with 80 g of 0.1 wt % vitamin E-blended GUR® 1050 UHMWPE. Controls were uniformly molded UHMWPEs blended with the same concentration of vitamin E and peroxide.

The cross-link density of the consolidated pucks was measured using small sections (approximately 3×3 mm sections from the first 1 mm for ‘surface’ and 3 mm cubes from the center for ‘bulk’, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density of the layered material at the surface was 257 mol/m³ for 0.5 wt % peroxide cross-linked sample and 299 mol/m³ for the 1 wt % peroxide cross-linked sample. The cross-link density of the uniformly blended samples was 250 mol/m³ for 0.5 wt % peroxide-blended UHMWPE and 301 mol/m³ for 1 wt % peroxide-blended UHMWPE.

The wear rate of peroxide cross-linked samples was measured by bidirectional pin-on-disc testing, as described above, with a 5 by 10 mm rectangular crossing pattern at 2 Hz for 1.2 million cycles. Wear was measured gravimetrically at 0.5 MC and at every 0.16 MC afterwards. The wear rate was calculated by the linear regression of the wear against number of cycles from 0.5 to 1.2 MC.

The wear rate of 0.1 wt % vitamin E-blended UHMWPE cross-linked using 1 wt % Luperox® 130 (P130) was 0.71±0.25 mg/MC in the surface cross-linked layered material and 0.32±0.15 mg/MC in the uniformly cross-linked sample.

Double notched IZOD impact testing was performed according to ASTM F648. The impact toughness of the layered material with 0.5 wt % peroxide-blended UHMWPE was 96.8±2.2 kJ/m² and that with 1 wt % peroxide-blended UHMWPE was 90.5±2.7 kJ/m² compared to 104.2±1.4 kJ/m² for the 0.1 wt % vitamin E-blended, uncross-linked UHMWPE, 76.0±0.6 kJ/m² for the 0.1 wt % vitamin E-blended UHMWPE uniformly cross-linked using 0.5 wt % P130 and 64.3±1.1 kJ/m² for the 0.1 wt % vitamin E-blended UHMWPE uniformly cross-linked using 1 wt % P130.

### Example 23

### Gamma Sterilization of Vitamin E-Blended UHMWPE Crosslinked by Using Peroxide Blending (P130 or Luperox® 130)

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. These blends were further mixed with the desired amount of the peroxide. The virgin UHMWPE/peroxide blends and the UHMWPE/antioxidant/peroxide blends were placed in a mold and they were consolidated into pucks (diameter 10 cm, thickness 1 cm) with the press platens at the desired temperature and pressure (20 MPa) for about 2 hours followed by a cool-down for about three hours to room temperature under pressure. At times, the pressure varied above 20 MPa up to 40 MPa during molding. The peroxide in this example was P130.

The vitamin E concentrations used were 0 wt %, 0.1 wt %, 0.2 wt %, 0.3 wt %, 0.5 wt %, 0.6 wt %, 0.8 wt % and 1 wt %. The peroxide concentrations used were 0.5 wt %, and 1 wt %. Molding was done at 190° C.

Then, the pucks were packaged in inert gas and gamma sterilized to a nominal dose of 25-40 kGy.

The cross-link density of the consolidated pucks was measured using small sections (approximately 3 mm cubes, n=6 each) prepared manually by cutting with a razor blade. The samples were placed in 25 mL of pre-heated xylene 130° C. in an oil bath and were allowed to swell for 2 hours. The dry sample weight and the swollen sample weight were measured in sealed containers before and after xylene immersion to determine a gravimetric swell ratio. The gravimetric swelling ratio was converted to a volumetric swelling ratio using the density of the dry polymer as 0.94 g/cm³ and the density of xylene at 130° C. as 0.75 g/cm³. The cross-link density of the samples (n=3 each) was calculated using the following equations:

$$\begin{matrix}{d}_{x} = \frac{ln ( 1 - {q}_{eq}^{- 1} ) + {q}_{eq}^{- 1} + {Xq}_{eq}^{- 2}}{{V}_{1} ( {q}_{eq}^{- 1 \text{/} 3} - {q}_{eq}^{- 2} )} & ( Eq . 1 ) \\ X = 0.33 + \frac{0.33}{{q}_{eq}} & ( Eq . 2 )\end{matrix}$$

where the specific volume of xylene, V₁, was 136 cm³/mol.

The cross-link density results are shown in Table 10 below. The numbers in parentheses in Table 10 are standard deviations.

TABLE 10
The cross-link density (mol/m³) of gamma sterilized, vitamin
E-blended UHMWPE cross-linked by Luperox ®-130 (P130)
by blending into powder and decomposing the peroxide during
compression molding as a function of peroxide concentration.

| | 0.5 wt % | 1 wt % |
|---|---|---|
| | P130 | P130 |
| 0.3 wt % Vitamin-E | 237 (5) | 288 (10) |
| 0.5 wt % Vitamin-E | 210 (4) | 276 (5) |
| 0.6 wt % Vitamin-E | 208 (5) | 270 (4) |
| 0.8 wt % Vitamin-E | 192 (4) | 255 (3) |
| 1 wt % Vitamin-E | 178 (4) | 241 (3) |

The results showed that highly cross-linked vitamin E-blended UHMWPE were achieved by peroxide cross-linking vitamin E blended UHMWPE during compression molding followed by gamma sterilization.

The wear rate of peroxide cross-linked samples was measured by bidirectional pin-on-disc testing with a 5 by 10 millimeter rectangular crossing pattern at 2 Hz for 1.2 million cycles. Wear was measured gravimetrically at 0.5 MC and at every 0.16 MC afterwards. The wear rate was calculated by the linear regression of the wear against number of cycles from 0.5 to 1.2 MC.

The wear rate results are shown in Table 11 below. The numbers in parentheses in Table 11 are standard deviations.

TABLE 11
The pin-on-disc wear rate (mg/million cycles) of
gamma sterilized vitamin E-blended UHMWPE cross-
linked by Luperox ®-130 (P130) during compression
molding as a function of peroxide concentration.

| | 0.5 wt % | 1 wt % |
|---|---|---|
| | P130 | P130 |
| 0.3 wt % Vitamin-E | 1.95 (0.2) | 0.5 (0.1) |
| 0.5 wt % Vitamin-E | 3.1 (0.1) | |
| 0.6 wt % Vitamin-E | 3.6 (0.3) | 1.1 (0.3) |
| 0.8 wt % Vitamin-E | 4.5 (0.2) | |
| 1 wt % Vitamin-E | 4.8 (0.6) | |

The results showed that low (1-2 mg/MC) and extremely low wear rates (<1 mg/MC) could be obtained using peroxide cross-linking of vitamin E-blended UHMWPE followed by gamma sterilization.

It is also important to note that in comparison with the samples of Example 16, gamma sterilization resulted in a reduction in cross-link density of the peroxide cross-linked vitamin E/UHMWPE blends while at the same time there wear rate decreased. This was an unexpected finding, as further cross-linking should occur with gamma sterilization. Also these findings were unexpected because of the fact that there was a decrease in the wear rate with a decrease in cross-link density.

Tensile testing was performed on Type V dogbones in accordance with ASTM D638. Thin sections (3.2 mm-thick) were machined from the peroxide cross-linked pucks, out of which dogbones were stamped. The dogbones were tested in tension at a crosshead speed of 10 mm/min (Insight 2, MTS, Eden Prairie, Minn., USA). The strain was measured by a laser extensometer.

The ultimate tensile strength (UTS) of peroxide cross-linked UHMWPEs are reported in Table 12 below. The numbers in parentheses in Table 12 are standard deviations.

TABLE 12
The ultimate tensile strength (MPa) of gamma sterilized vitamin
E-blended UHMWPE cross-linked by Luperox ®-130 (P130)
by blending into powder and decomposing the peroxide during
compression molding as a function of peroxide concentration.

| | 0.5 wt % | 1 wt % |
|---|---|---|
| | P130 | P130 |
| 0.3 wt % Vitamin-E | 37.7 (5.2) | 31.9 (3.3) |
| 0.5 wt % Vitamin-E | 44.5 (2.9) | 36.4 (2.4) |
| 0.6 wt % Vitamin-E | 44.5 (4.2) | 32.6 (6.8) |
| 0.8 wt % Vitamin-E | 47.5 (3.3) | 39.6 (2.7) |
| 1 wt % Vitamin-E | 45.0 (4.2) | 41.1 (2.6) |

The elongation-at-break (EAB) of gamma sterilized, peroxide cross-linked UHMWPEs are shown in Table 13. The numbers in parentheses in Table 13 are standard deviations.

TABLE 13
The elongation at break (%) of gamma sterilized vitamin E-
blended UHMWPE cross-linked by Luperox ®-130 (P130)
by blending into powder and decomposing the peroxide during
compression molding as a function of peroxide concentration.

| | 0.5 wt % | 1 wt % |
|---|---|---|
| | P130 | P130 |
| 0.3 wt % Vitamin-E | 315 (20) | 249 (12) |
| 0.5 wt % Vitamin-E | 306 (17) | 281 (7) |
| 0.6 wt % Vitamin-E | 317 (17) | 248 (41) |
| 0.8 wt % Vitamin-E | 324 (30) | 291 (7) |
| 1 wt % Vitamin-E | 340 (16) | 320 (20) |

Cubes (10 mm) were machined from the gamma sterilized, peroxide cross-linked blends. Then, the cubes were doped with squalene at 110° C. for 1 hour and after cooling, were placed in a pressure vessel at 70° C. at 5 atm. of oxygen for 14 days. Oxidation profiles were determined by using FTIR. The cubes were cut in half, 150 μm cross sections were microtomed from the inner surfaces. These thin sections were extracted by boiling hexane for 16 hours and dried under vacuum for 24 hours. These were then analyzed on an infrared microscope (BioRad UMA 500, Bio-Rad, Cambridge, Mass., USA) as a function of depth away from the surface. An oxidation index was calculated, as described above, as the ratio of the area under the carbonyl peaks at 1700 cm⁻¹ to the area under the methylene vibration at 1370 cm⁻¹ peak. Measurements were made on three separate thin sections. An average surface oxidation index was calculated as an average of the surface 1.5 mm of the samples.

The average amount of squalene absorbed into cross-linked UHMWPEs was 21 mg as measured after doping and before aging. The average surface oxidation index was below 0.03 for all tested samples after accelerated aging (0.5 wt % vitamin E/0.5 wt % peroxide; 0.6 wt % vitamin E/0.5 wt % peroxide; 0.8 wt % vitamin E/0.5 wt % peroxide; 1.0 wt % vitamin E/0.5 wt % peroxide). This suggested that these were all extremely resistant against oxidation.

### Example 24

### IZOD Impact Testing of Vitamin E-Blended UHMWPE by Using Peroxide Blending (P130 or Luperox® 130)

Vitamin E was blended with GUR® 1050 UHMWPE powder with the aid of isopropyl alcohol (IPA) as described in Example 1. The solvent was substantially removed from the polymer blend by evaporation. A master batch was prepared containing 2 wt % vitamin E. Lower concentration blends were prepared by diluting the master batch down to the desired vitamin E concentration by blending with virgin UHMWPE as needed. These blends were further mixed with the desired amount of the peroxide. The virgin UHMWPE/peroxide blends and the UHMWPE/antioxidant/peroxide blends were placed in a mold and they were consolidated into pucks (diameter 10 cm, thickness 1 cm) with the press platens at the desired temperature and pressure (20 MPa) for about 2 hours followed by a cool-down for about three hours to room temperature under pressure.

The vitamin E concentrations used were 0.5 wt %, 0.6 wt % and 0.8 wt %. The peroxide (P-130) concentrations used were 0.5 wt %, 1 wt % or 1.5 wt %. Molding was done at 190° C.

Double notched IZOD impact testing was performed according to ASTM F648. The results are shown in Table 14. The numbers in parentheses in Table 14 are standard deviations.

TABLE 14
The IZOD impact strength (kJ/m²) of gamma vitamin E-blended
UHMWPE cross-linked by Luperox ®-130 (P130) by blending
into powder and decomposing the peroxide during compression
molding as a function of peroxide concentration.

| | | |
|---|---|---|
| 0.5 wt % | 1 wt % | 1.5 wt % |
| P130 | P130 | P130 |

GUR1050

| | | | |
|---|---|---|---|
| 0.5 wt % | | | 60.6 (0.6) |
| Vitamin-E | | | |
| 0.6 wt % | | | 61.8 (0.5) |
| Vitamin-E | | | |
| 0.8 wt % | 91.1 (0.3) | 77.1 (0.5) | 65.6 (0.5) |
| Vitamin-E | | | |

GUR1020

| | | | |
|---|---|---|---|
| 0.5 wt % | | | 63.0 (0.3) |
| Vitamin-E | | | |
| 0.6 wt % | | | 64.0 (0.9) |
| Vitamin-E | | | |
| 0.8 wt % | 97.2 (0.8) | 80.4 (0.8) | 68.3 (0.5) |
| Vitamin-E | | | |

The results of impact testing showed that GUR1020 had slightly higher impact strength compared to GUR1050 at the same vitamin E and peroxide concentration.

### INDUSTRIAL APPLICABILITY

This invention provides methods of chemically cross-linking antioxidant-stabilized polymeric material.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

Each reference identified in the present application is herein incorporated by reference in its entirety.

1-194. (canceled)

195. A method of making an oxidation resistant, cross-linked polymeric material, the method comprising:
(a) blending a polymeric material with an antioxidant and a cross-linking agent such that the antioxidant is present in the polymeric material at a concentration of from 0.1 to 2 wt % by weight of the polymeric material and such that the cross-linking agent is present in the polymeric material at a concentration of from 0.5 to 2 wt % by weight of the polymeric material; and
(b) consolidating the polymeric material thereby forming a consolidated, antioxidant and cross-linking agent-blended polymeric material.

196. The method of claim 195 wherein the antioxidant and cross-linking agent-blended polymeric material is further heated, wherein:
the heating is done to a temperature T at about or above (i) a temperature T₁ at which one-half of a quantity of the peroxide decomposes in one hour, or (ii) a temperature T₁₀ at which one-half of a quantity of the peroxide decomposes in ten hour, or (iii) at about above the melting point of the polymeric material.

197. The method of claim 196 wherein:
the heating is done in the range of about 180° C. to about 300° C. for 30 minutes to 48 hours or longer.

198. The method of claim 196 wherein:
step (b) and the heating are done concurrently.

199. The method of claim 195 further comprising:
machining the oxidation resistant, cross-linked polymeric material into a medical implant, packaging and sterilizing the medical implant, wherein sterilizing is done by gas sterilization or ionizing irradiation.

200. The method of claim 195, wherein:
the polymeric material is selected from ultrahigh molecular weight polyethylenes, high density polyethylene, low density polyethylene, linear low density polyethylene, and mixtures and blends thereof.

201. The method of claim 195, wherein:
the cross-linking agent comprises a peroxide selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof, and
the antioxidant is selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

202. The method of claim 195 wherein:
step (b) comprises compression molding or direct compression molding the polymeric material to a second surface, such as a porous metal, thereby making an interlocked hybrid material.

203. The method of claim 196 further comprising machining the polymeric material before or after heating.

204. A method of making an oxidation resistant, cross-linked polymeric material, the method comprising:
(a) blending a first polymeric material with an antioxidant and a cross-linking agent such that the antioxidant is present in the first polymeric material at a concentration of from 0.05 to 2 wt % by weight of the first polymeric material and such that the cross-linking agent is present in the first polymeric material at a concentration of from 0.5 to 2 wt % by weight of the first polymeric material;
(b) blending a second polymeric material with an antioxidant and a cross-linking agent such that the antioxidant is present in the second polymeric material at a concentration of from 0.05 to 2 wt % by weight of the second polymeric material and such that the cross-linking agent is present in the second polymeric material at a concentration of from 0.0 to 2 wt % by weight of the second polymeric material; and
(c) consolidating the first polymeric material and the second polymeric material in layers thereby forming a consolidated, antioxidant and cross-linking agent-blended polymeric material; and,
wherein levels of crosslinking are different in a first layer and a second layer of the layers.

205. The method according to claim 204, wherein the consolidated, antioxidant and cross-linking agent-blended polymeric material is further heated wherein,
the heating is done to a temperature T at about or above (i) a temperature T₁ at which one-half of a quantity of the peroxide decomposes in one hour, or (ii) a temperature T₁₀ at which one-half of a quantity of the peroxide decomposes in ten hour, or (iii) at about above the melting point of the polymeric material.

206. The method of claim 204, wherein:
the cross-linking agent comprises a peroxide selected from inorganic peroxides, diacyl peroxides, peroxyesters, peroxydicarbonates, dialkyl peroxides, ketone peroxides, peroxyketals, cyclic peroxides, peroxymonocarbonates, hydroperoxides, dicumyl peroxide, benzoyl peroxide, 2,5-Di(tert-butylperoxy)-2,5-dimethyl-3-hexyne, 3,3,5,7,7-pentamethyl 1,2,4-trioxepane, dilauryl peroxide, methyl ether ketone peroxide, t-amyl peroxyacetate, t-butyl hydroperoxide, t-amyl peroxybenzoate, D-t-amyl peroxide, 2,5-Dimethyl 2,5-Di(t-butylperoxy)hexane, t-butylperoxy isopropyl carbonate, succinic acid peroxide, cumene hydroperoxide, 2,4-pentanedione peroxide, t-butyl perbenzoate, diethyl ether peroxide, acetone peroxide, arachidonic acid 5-hydroperoxide, carbamide peroxide, tert-butyl hydroperoxide, t-butyl peroctoate, t-butyl cumyl peroxide, Di-sec-butyl-peroxydicarbonate, D-2-ethylhexylperoxydicarbonate, 1,1-Di(t-butylperoxy)cyclohexane, 1,1-Di(tert-butylperoxy)-3,3,5-trimethylcyclohexane, 2,5-Dimethyl-2,5-di(tert-butylperoxy)hexane, 3,3,5,7,7-Pentamethyl-1,2,4-trioxepane, Butyl 4,4-di(tert-butylperoxy)valerate, Di(2,4-dichlorobenzoyl) peroxide, Di(4-methylbenzoyl) peroxide, Di(tert-butylperoxyisopropyl)benzene, tert-Butyl cumyl peroxide, tert-Butyl peroxy-3,5,5-trimethylhexanoate, tert-Butyl peroxybenzoate, tert-Butylperoxy 2-ethylhexyl carbonate, and mixtures thereof, and
the antioxidant is selected from glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox® family; Irganox® and Irganox® B families including Irganox® 1010, Irganox® 1076, Irganox® 1330, Irganox® 1035; Irgafos® family; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, and mixtures, derivatives, analogues or conjugated forms of these.

207. The method of claim 204 wherein:
step (c) comprises compression molding at least one of the first polymeric material and the second polymeric material to a second surface, such as a porous metal, thereby making an interlocked hybrid material.

208. The method of claim 204 further comprising machining the polymeric material before or after heating.

209. The method of claim 204 further comprising machining the consolidated polymeric material into a medical implant, packaging and sterilizing the medical implant, wherein sterilizing is done by gas sterilization or ionizing irradiation.

210. The method of claim 205 wherein:
the heating is done in the range of about 180° C. to about 300° C. for 30 minutes to 48 hours or longer.
